(19) United States
(12) Patent Application Publication
(10) Pub. No.: US 2017/0173037 A1
(43) Pub. Date: Jun. 22, 2017

(54) PHARMACEUTICAL COMPOSITIONS

(71) Applicant: CHARLESTON LABORATORIES, INC., Jupiter, FL (US)

(72) Inventors: Paul BOSSE, Jupiter, FL (US); John AMELING, Jupiter, FL (US); William KOZAREK, Jensen Beach, FL (US); Bernard SCHACHTEL, Jupiter, FL (US)

(21) Appl. No.: 15/452,628

(22) Filed: Mar. 7, 2017

Related U.S. Application Data
(63) Continuation of application No. PCT/US15/48999, filed on Sep. 8, 2015.
(60) Provisional application No. 62/047,882, filed on Sep. 9, 2014.
(60) Provisional application No. 62/168,334, filed on May 29, 2015.

(51) Int. Cl. A61K 31/5415; A61K 9/48; A61K 9/00; A61K 31/4045; A61K 9/51

(52) U.S. Cl. CPC A61K 31/5415; A61K 31/4045; A61K 9/5161; A61K 9/5138; A61K 9/5123; A61K 9/0053; A61K 9/4808

(57) ABSTRACT

Pharmaceutical compositions and methods are provided to treat headache, headache-associated symptoms, photophobia, or adverse effects associated with triptan administration.

### CROSS-REFERENCE

This application is a Continuation of PCT/US15/48999 filed Sep. 8, 2015, which claims the benefit of U.S. Provisional Application No. 62/047,882, filed on Sep. 9, 2014, and U.S. Provisional Application No. 62/168,334, filed on May 29, 2015, all of which are incorporated herein by reference in their entirety.

### BACKGROUND

Available pain medications are typically provided in individual doses. The therapeutic effect of these medications may be improved by combining them with other medications capable of providing pain relief. Additionally, available pain medications may have adverse effects, such as nausea and vomiting. As a result of such adverse effects, many subjects are unable to tolerate recommended dosages needed for effective pain relief. Accordingly, combination therapies may also address the need for effective therapeutics with reduced adverse effects.

### BRIEF SUMMARY

In some aspects, a pharmaceutical composition is provided, the pharmaceutical composition comprising a plurality of first particulates comprising a 5HT_{1B} receptor agonist or a pharmaceutically acceptable salt thereof, and a plurality of second particulates comprising an antiemetic or a pharmaceutically acceptable salt thereof, wherein a weight ratio of the plurality of first particulates to the plurality of second particulates is of from about 3:1 to about 5:1. In some instances, a weight ratio of the 5HT_{1B} receptor agonist or a pharmaceutically acceptable salt thereof to the antiemetic or a pharmaceutically acceptable salt thereof is of from about 1:2 to about 15:1. In some instances, the weight ratio of the 5HT_{1B} receptor agonist or a pharmaceutically acceptable salt thereof to the antiemetic or a pharmaceutically acceptable salt thereof is of from about 3:2 and about 11:1. In some instances, the weight ratio of the 5HT_{1B} receptor agonist or a pharmaceutically acceptable salt thereof to the antiemetic or a pharmaceutically acceptable salt thereof is of from about 3:1 and about 7:1. In some instances, the weight ratio of the 5HT_{1B} receptor agonist or a pharmaceutically acceptable salt thereof to the antiemetic or a pharmaceutically acceptable salt thereof is of from about 9:2 and about 11:2. In some instances, the weight ratio of the 5HT_{1B} receptor agonist or a pharmaceutically acceptable salt thereof to the antiemetic or a pharmaceutically acceptable salt thereof is about 5:1. In some instances, the weight ratio of the plurality of the first particulates to the plurality of the second particulates is of from about 5:1 to about 3:1, for example about 4:1. In some instances, a weight ratio of the 5HT_{1B} receptor agonist or the pharmaceutically acceptable salt thereof to the total weight of the plurality of the first particulates is of from about 2:5 to about 7:10. In some instances, a weight ratio of the antiemetic or a pharmaceutically acceptable salt thereof to the total weight of the plurality of the second particulates is of from about 2:5 to about 3:5. In some instances, the plurality of the first particulates comprises one or more first pharmaceutically acceptable excipients and a weight ratio of the total amount of the 5HT_{1B} receptor agonist or pharmaceutically acceptable salt thereof to the total amount of the one or more first pharmaceutically acceptable excipients is of from about 2:1 to about 1:1, for example about 3:2. In some instances, the plurality of the second particulates comprises one or more second pharmaceutically acceptable excipients, and a weight ratio of the total amount of the antiemetic or a pharmaceutically acceptable salt thereof to the total amount of the one or more second pharmaceutically acceptable excipients is of from about 2:1 to about 1:2, for example about 1:1. In some instances, the 5HT_{1B} receptor agonist is present in an amount of from about 50% to about 70% by weight of the plurality of the first particulates. In some instances, the 5HT_{1B} receptor agonist is present in an amount of about 61% by weight of the plurality of the first particulates. In some instances, the antiemetic or a pharmaceutically acceptable salt thereof is present in an amount of from about 40% to about 60% by weight of the plurality of the second particulates. In some instances, the antiemetic or a pharmaceutically acceptable salt thereof is present in an amount of about 50% by weight of the plurality of the second particulates. In some instances, about 90% to about 100% of the 5HT_{1B} receptor agonist or a pharmaceutically acceptable salt thereof is stable for at least 30 days as measured by HPLC. In some instances, about 90% to about 100% of the antiemetic or a pharmaceutically acceptable salt thereof is stable for at least 30 days as measured by HPLC. In some instances, a diameter of each of the first particulates is of from about 595 microns to about 1190 microns. In some instances, a diameter of each of the second particulates is of from about 595 microns to about 1190 microns. In some instances, a diameter of each of the first particulates is of from about 595 microns to about 1190 microns, and a diameter of each of the second particulates is of from about 595 microns to about 1190 microns. In some instances, the 5HT_{1B} receptor agonist or a pharmaceutically acceptable salt thereof comprises a triptan or a pharmaceutically acceptable salt thereof. In some instances, the triptan or a pharmaceutically acceptable salt thereof comprises sumatriptan or a pharmaceutically acceptable salt thereof. In some instances, the sumatriptan is present in an amount of about 25 mg to about 100 mg. In some instances, the sumatriptan is present in an amount of about 90 mg. In some instances, the pharmaceutically acceptable salt of sumatriptan comprises sumatriptan succinate. In some instances, the sumatriptan succinate is present in an amount of from about 35 mg to about 140 mg. In some instances, the sumatriptan succinate is present in an amount of about 126 mg. In some instances, the pharmaceutically acceptable salt of sumatriptan is present in an amount therapeutically equivalent to about 90 mg of sumatriptan. In some instances, the antiemetic or a pharmaceutically acceptable salt thereof comprises promethazine or a pharmaceutically acceptable salt thereof. In some instances, the promethazine is present in an amount of about 12.5 mg to about 50 mg. In some instances, the promethazine is present in an amount of about 22 mg. In some instances, the pharmaceutically acceptable salt of promethazine comprises promethazine hydrochloride. In some instances, the promethazine hydrochloride is present in an amount of from about 5 to about 50 mg, e.g., about 25 mg. In some instances, the pharmaceutically acceptable salt of promethazine is present in an amount therapeutically equivalent to about 22 mg of promethazine. In some instances, the pharmaceutically acceptable salt of the 5HT_{1B} receptor agonist comprises triptan succinate and the triptan base is present in an amount of about 90 mg. In some instances, the pharmaceutically acceptable salt of the 5HT_{1B} receptor agonist comprises triptan succinate and the triptan base is present in an amount of about 100 mg. In some instances, the pharmaceutically acceptable salt of the 5HT_{1B} receptor agonist comprises sumatriptan succinate and the sumatriptan base is present in an amount of about 90 mg. In some instances, the pharmaceutically acceptable salt of the 5HT_{1B} receptor agonist comprises sumatriptan succinate and the sumatriptan base is present in an amount of about 100 mg. In some instances, the pharmaceutically acceptable salt of the antiemetic comprises promethazine hydrochloride and the promethazine hydrochloride is present in an amount of about 25 mg. In some instances, the pharmaceutical composition is in an oral dosage form. In some instances, that the oral dosage form comprises a capsule. In some instances, the pharmaceutical composition is housed within a container. In some instances, the container is a bottle or pill blister. In some aspects, a pharmaceutical composition disclosed herein for use in treatment of a headache in a subject in need thereof. In some instances, the pharmaceutical composition is for use in treatment of a headache, wherein the treatment is acute. In some instances, the pharmaceutical composition is for use in treatment of a headache, wherein the treatment is prophylactic. In some instances, the pharmaceutical composition is for use in treatment of a migraine headache. In some instances, the pharmaceutical composition is for use in treatment of an acute migraine headache. In some instances, the pharmaceutical composition is for use in treatment of a chronic migraine headache. In some instances, the pharmaceutical composition is for use in treatment of a migraine headache with or without an aura. In some instances, the pharmaceutical composition is for use in treatment of a cluster headache. In some instances, the pharmaceutical composition is for use in treatment of nausea or vomiting. In some instances, the pharmaceutical composition is for use in treatment of nausea associated with a headache or vomiting associated with a headache. In some instances, the pharmaceutical composition is for use in treatment headache and vomiting associated with a headache. In some aspects, a pharmaceutical composition disclosed herein for use in treatment of a photophobia in a subject in need thereof. In some instances, the pharmaceutical composition is for use in treatment of a photophobia, wherein the treatment is acute. In some instances, the pharmaceutical composition is for use in treatment of a photophobia, wherein the treatment is prophylactic. In some instances, the pharmaceutical composition is for use in treatment of a light sensitivity. In some instances, the pharmaceutical composition is for use in treatment of nausea or vomiting. In some instances, the pharmaceutical composition is for use in treatment of nausea associated with a headache or vomiting associated with a headache. In some instances, the pharmaceutical composition is for use in treatment headache and vomiting associated with a headache. In some instances, the triptan or a pharmaceutically acceptable salt thereof disclosed herein comprises sumatriptan, almotriptan, frovatriptan, eletriptan, rizatriptan, naratriptan, or a pharmaceutically acceptable salt thereof. In some instances, the antiemetic or a pharmaceutically acceptable salt thereof comprises promethazine, ondansetron, aprepitant, dronabinol, perphenazine, palonosetron, trimethyobenzamide, metoclopromide, domperidone, prochlorperazine, chlorpromazine, trimethobenzamide, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, or a pharmaceutically acceptable salt thereof.

In some instances, at least about 80% of both the 5HT_{1B} receptor agonist or a pharmaceutically acceptable salt thereof and the antiemetic are released within about 15 minutes as measured by contact of the pharmaceutical composition with dissolution fluid in a USP Apparatus 1 (Basket) rotating at 100 rpm. In some instances, the antiemetic or a pharmaceutically acceptable salt thereof has a slower release rate than the release rate of the 5HT_{1B} receptor agonist or a pharmaceutically acceptable salt thereof. In some instances, the 5HT_{1B} receptor agonist or a pharmaceutically acceptable salt thereof comprises a triptan or a pharmaceutically acceptable salt thereof. In some instances, the triptan or a pharmaceutically acceptable salt thereof comprises sumatriptan or a pharmaceutically acceptable salt thereof. In some instances, the sumatriptan is present in an amount of about 25 mg to about 100 mg. In some instances, the sumatriptan is present in an amount of about 90 mg. In some instances, the pharmaceutically acceptable salt of sumatriptan comprises sumatriptan succinate. In some instances, the sumatriptan succinate is present in an amount of from about 35 mg to about 140 mg. In some instances, the sumatriptan succinate is present in an amount of about 126 mg. In some instances, the pharmaceutically acceptable salt of sumatriptan is present in an amount therapeutically equivalent to about 90 mg of sumatriptan. In some instances, the antiemetic or a pharmaceutically acceptable salt thereof comprises promethazine or a pharmaceutically acceptable salt thereof. In some instances, the promethazine is present in an amount of about 12.5 mg to about 50 mg. In some instances, the promethazine is present in an amount of about 22 mg. In some instances, the pharmaceutically acceptable salt of promethazine comprises promethazine hydrochloride. In some instances, the promethazine hydrochloride is present in an amount of from about 5 to about 50 mg, e.g., about 25 mg. In some instances, the pharmaceutically acceptable salt of promethazine is present in an amount therapeutically equivalent to about 22 mg of promethazine. In some instances, a total weight of the plurality of first particulates is of from about 175 mg to about 300 mg. In some instances, the plurality of first particulates is of from about 200 mg to about 220 mg. In some instances, the total weight of the plurality of first particulates is of from about 208 mg to about 212 mg. In some instances, a total weight of the plurality of second particulates is of from about 30 mg to about 100 mg. In some instances, the total weight of the plurality of second particulates is of from about 45 mg to about 55 mg. In some instances, the total weight of the plurality of second particulates is of about 50 mg or about 51 mg. In some instances, the plurality of first particulates comprises one or more first pharmaceutically acceptable excipients, wherein the one or more first pharmaceutically acceptable excipients comprises a diluent, binder, disintegrant or lubricant. In some instances, the diluent comprises microcrystalline cellulose. In some instances, the binder comprises polyvinylpyrrolidone. In some instances, the disintegrant comprises croscarmellose sodium. In some instances, the lubricant comprises magnesium stearate or talc. In some instances, the plurality of second particulates comprises one or more first pharmaceutically acceptable excipients, wherein the one or more first pharmaceutically acceptable excipients comprises a diluent or a disintegrant. In some instances, the diluent comprises microcrystalline cellulose. In some instances, the disintegrant comprises croscarmellose sodium. In some instances, the plurality of first particulates comprises about 50-150 mg of the 5HT_{1B} receptor agonist or a pharmaceutically acceptable salt thereof, about 1-10 mg of polyvinylpyrrolidone, about 50-100 mg of microcrystalline cellulose, about 1-10 mg of croscarmellose sodium, about 0.1-5 mg of magnesium stearate, and a coating material; and the plurality of second particulates comprises about 10-50 mg of antiemetic or a pharmaceutically acceptable salt thereof, about 10-50 mg of microcrystalline cellulose, about 0.1-5 mg of croscarmellose sodium and a coating material. In some instances, the plurality of first particulates comprises about 90 mg of sumatriptan or a therapeutically equivalent amount of pharmaceutically acceptable salt thereof, about 4 mg of polyvinylpyrrolidone, about 69 mg of microcrystalline cellulose, about 4 mg of croscarmellose sodium, about 1 mg of magnesium stearate and a coating material, wherein the coating material comprises polyvinyl alcohol; and the plurality of second particulates comprises about 22 mg of promethazine or a therapeutically equivalent amount of pharmaceutically acceptable salt thereof, about 24 mg of microcrystalline cellulose, about 1 mg of croscarmellose sodium; and a coating material, wherein the coating material comprises polyvinyl alcohol. In some instances, the plurality of first particulates comprises from about 40% to about 80% by weight of the 5HT_{1B} receptor agonist or a pharmaceutically acceptable salt thereof, from about 0.5% it about 5% by weight of polyvinylpyrrolidone, from about 20% to about 60% by weight of microcrystalline cellulose, from about 0.5% to about 5% by weight of croscarmellose sodium, from about 0.1% to about 5% by weight of magnesium stearate and a coating material; and

the plurality of second particulates comprises from about 30% to about 70% by weight of the antiemetic or a pharmaceutically acceptable salt thereof, from about 20% to about 70% by weight of microcrystalline cellulose, from about 0.5% to about 5% by weight of croscarmellose sodium and a coating material. In some instances, the plurality of first particulates comprises about 60.5% by weight of sumatriptan succinate, about 2% by weight of polyvinylpyrrolidone, about 35% by weight of microcrystalline cellulose, about 2% by weight of croscarmellose sodium, about 0.5% by weight of magnesium stearate and a coating material, wherein the coating material comprises polyvinyl alcohol; and the plurality of second particulates comprises about 50% by weight of promethazine hydrochloride, about 48% by weight of microcrystalline cellulose, about 2% by weight of croscarmellose sodium, and a coating material, wherein the coating material comprises polyvinyl alcohol. In some instances, the first particulates comprise a coating material. In some instances, the coating material is applied to the plurality of the first particulates at a weight gain of from about 0.5% to about 5%, for example about 2%. In some instances, the second particulates comprise a coating material. In some instances, the coating material is applied to the plurality of the second particulates at a weight gain of from about 0.5% to about 5%, for example about 2%. In some instances, the first particulates and the second particulates comprise the same coating material. In some instances, the coating material comprises polyvinyl alcohol, cellulose acetate phthalate, polyvinyl acetate phthalate, methacrylic acid copolymer, cellulose acetate trimellitate, hydroxypropyl methylcellulose phthalate, hydroxypropyl methylcellulose, hydroxypropyl methyl cellulose acetate succinate, shellac, sodium alginate or zein. In some instances, the coating material comprises polyvinyl alcohol. In some instances, the coating material is polyvinyl alcohol. In some instances, a diameter of each of the first particulates is of from about 595 microns to about 1190 microns. In some instances, a diameter of each of the second particulates is of from about 595 microns to about 1190 microns. In some instances, a diameter of each of the first particulates is of from about 595 microns to about 1190 microns, and a diameter of each of the second particulates is of from about 595 microns to about 1190 microns. In some instances, the pharmaceutically acceptable salt of the 5HT_{1B} receptor agonist comprises triptan succinate and the triptan base is present in an amount of about 90 mg. In some instances, the pharmaceutically acceptable salt of the 5HT_{1B} receptor agonist comprises triptan succinate and the triptan base is present in an amount of about 100 mg. In some instances, the pharmaceutically acceptable salt of the 5HT_{1B} receptor agonist comprises sumatriptan succinate and the sumatriptan base is present in an amount of about 90 mg. In some instances, the pharmaceutically acceptable salt of the 5HT_{1B} receptor agonist comprises sumatriptan succinate and the sumatriptan base is present in an amount of about 100 mg. In some instances, the pharmaceutically acceptable salt of the antiemetic comprises promethazine hydrochloride and the promethazine hydrochloride is present in an amount of about 25 mg. In some instances, the pharmaceutical composition is in an oral dosage form. In some instances, that the oral dosage form comprises a capsule. In some instances, the pharmaceutical composition is housed within a container. In some instances, the container is a bottle or pill blister. In some aspects, a pharmaceutical composition disclosed herein for use in treatment of a headache in a subject in need thereof. In some instances, the pharmaceutical composition is for use in treatment of a headache wherein the treatment is acute. In some instances, the pharmaceutical composition is for use in treatment of a headache wherein the treatment is prophylactic. In some instances, the pharmaceutical composition is for use in treatment of a migraine headache. In some instances, the pharmaceutical composition is for use in treatment of an acute migraine headache. In some instances, the pharmaceutical composition is for use in treatment of a chronic migraine headache. In some instances, the pharmaceutical composition is for use in treatment of a migraine headache with or without an aura. In some instances, the pharmaceutical composition is for use in treatment of a cluster headache. In some instances, the pharmaceutical composition is for use in treatment of nausea or vomiting. In some instances, the pharmaceutical composition is for use in treatment of nausea associated with a headache or vomiting associated with a headache. In some instances, the pharmaceutical composition is for use in treatment headache and vomiting associated with a headache. In some aspects, a pharmaceutical composition disclosed herein for use in treatment of a photophobia in a subject in need thereof. In some instances, the pharmaceutical composition is for use in treatment of a photophobia wherein the treatment is acute. In some instances, the pharmaceutical composition is for use in treatment of a photophobia wherein the treatment is prophylactic. In some instances, the pharmaceutical composition is for use in treatment of a light sensitivity. In some instances, the pharmaceutical composition is for use in treatment of nausea or vomiting. In some instances, the pharmaceutical composition is for use in treatment of nausea associated with a headache or vomiting associated with a headache. In some instances, the pharmaceutical composition is for use in treatment headache and vomiting associated with a headache. In some instances, the triptan or a pharmaceutically acceptable salt thereof disclosed herein comprises sumatriptan, almotriptan, frovatriptan, eletriptan, rizatriptan, naratriptan, or a pharmaceutically acceptable salt thereof. In some instances, the antiemetic or a pharmaceutically acceptable salt thereof comprises promethazine, ondansetron, aprepitant, dronabinol, perphenazine, palonosetron, trimethyobenzamide, metoclopromide, domperidone, prochlorperazine, chlorpromazine, trimethobenzamide, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, or a pharmaceutically acceptable salt thereof.

In some aspects, a pharmaceutical composition is provided, the pharmaceutical composition comprising a plurality of first particulates comprising a 5HT_{1B} receptor agonist or a pharmaceutically acceptable salt thereof, and a plurality of second particulates comprising an antiemetic or a pharmaceutically acceptable salt thereof, wherein at least about 80% of both the 5HT_{1B} receptor agonist or a pharmaceutically acceptable salt thereof and the antiemetic are released within about 15 minutes as measured by contact of the pharmaceutical composition with dissolution fluid in a USP Apparatus 1 (Basket) rotating at 100 rpm. In some instances, at least about 80% of both the 5HT_{1B} receptor agonist or a pharmaceutically acceptable salt thereof and the antiemetic or a pharmaceutically acceptable salt thereof are released within about 30 minutes as measured by contact of the pharmaceutical composition with a dissolution fluid in a USP Apparatus 1 (Basket) rotating at 100 rpm. In some instances, the antiemetic or a pharmaceutically acceptable salt thereof has about the same release rate as that of the 5HT_{1B} receptor agonist or a pharmaceutically acceptable salt thereof. In some instances, the antiemetic or a pharmaceutically acceptable salt thereof has about the same release rate as that of the 5HT_{1B} receptor agonist or a pharmaceutically acceptable salt thereof within about 15 minutes as measured by contact of the pharmaceutical composition with a dissolution fluid in a USP Apparatus 1 (Basket) rotating at 100 rpm. In some instances, the antiemetic or a pharmaceutically acceptable salt thereof has a slower release rate than the release rate of the 5HT_{1B} receptor agonist or a pharmaceutically acceptable salt thereof. In some instances, the antiemetic or a pharmaceutically acceptable salt thereof has a slower release rate than the release rate of the 5HT_{1B} receptor agonist or a pharmaceutically acceptable salt thereof within about 5 minutes as measured by contact of the pharmaceutical composition with a dissolution fluid in a USP Apparatus 1 (Basket) rotating at 100 rpm. In some instances, about 60% to about 65% of the antiemetic or a pharmaceutically acceptable salt thereof is released within about 5 minutes and about 70% to about 75% of the 5HT_{1B} receptor agonist or a pharmaceutically acceptable salt thereof is released within about 5 minutes as measured by contact of the pharmaceutical composition with a dissolution fluid in a USP Apparatus 1 (Basket) rotating at 100 rpm. In some instances, the pharmaceutical composition is a fast release pharmaceutical composition. In some instances, a weight ratio of the plurality of first particulates to the plurality of second particulates is of from about 3:1 to about 5:1. In some instances, the weight ratio of the 5HT_{1B} receptor agonist or a pharmaceutically acceptable salt thereof to the antiemetic or a pharmaceutically acceptable salt thereof is of from about 1:2 to about 15:1. In some instances, about 90% to about 100% of the 5HT_{1B} receptor agonist or a pharmaceutically acceptable salt thereof is stable for at least 30 days as measured by HPLC. In some instances, about 90% to about 100% of the antiemetic or a pharmaceutically acceptable salt thereof is stable for at least 30 days as measured by HPLC. In some instances, the 5HT_{1B} receptor agonist or a pharmaceutically acceptable salt thereof comprises a triptan or a pharmaceutically acceptable salt thereof. In some instances, the triptan or a pharmaceutically acceptable salt thereof comprises sumatriptan or a pharmaceutically acceptable salt thereof. In some instances, the sumatriptan is present in an amount of about 25 mg to about 100 mg. In some instances, the sumatriptan is present in an amount of about 90 mg. In some instances, the pharmaceutically acceptable salt of sumatriptan comprises sumatriptan succinate. In some instances, the sumatriptan succinate is present in an amount of from about 35 mg to about 140 mg. In some instances, the sumatriptan succinate is present in an amount of about 126 mg. In some instances, the pharmaceutically acceptable salt of sumatriptan is present in an amount therapeutically equivalent to about 90 mg of sumatriptan. In some instances, the antiemetic or a pharmaceutically acceptable salt thereof comprises promethazine or a pharmaceutically acceptable salt thereof. In some instances, the promethazine is present in an amount of about 12.5 mg to about 50 mg. In some instances, the promethazine is present in an amount of about 22 mg. In some instances, the pharmaceutically acceptable salt of promethazine comprises promethazine hydrochloride. In some instances, the promethazine hydrochloride is present in an amount of from about 5 to about 50 mg, e.g., about 25 mg. In some instances, the pharmaceutically acceptable salt of promethazine is present in an amount therapeutically equivalent to about 22 mg of promethazine. In some instances, a total weight of the plurality of first particulates is of from about 175 mg to about 300 mg. In some instances, the plurality of first particulates is of from about 200 mg to about 220 mg. In some instances, the total weight of the plurality of first particulates is of from about 208 mg to about 212 mg. In some instances, a total weight of the plurality of second particulates is of from about 30 mg to about 100 mg. In some instances, the total weight of the plurality of second particulates is of from about 45 mg to about 55 mg. In some instances, the total weight of the plurality of second particulates is of about 50 mg or about 51 mg. In some instances, the plurality of first particulates comprises one or more first pharmaceutically acceptable excipients, wherein the one or more first pharmaceutically acceptable excipients comprises a diluent, binder, disintegrant or lubricant. In some instances, the diluent comprises microcrystalline cellulose. In some instances, the binder comprises polyvinylpyrrolidone. In some instances, the disintegrant comprises croscarmellose sodium. In some instances, the lubricant comprises magnesium stearate or talc. In some instances, the plurality of second particulates comprises one or more first pharmaceutically acceptable excipients, wherein the one or more first pharmaceutically acceptable excipients comprises a diluent or a disintegrant. In some instances, the diluent comprises microcrystalline cellulose. In some instances, the disintegrant comprises croscarmellose sodium. In some instances, the plurality of first particulates comprises about 50-150 mg of the 5HT_{1B} receptor agonist or a pharmaceutically acceptable salt thereof, about 1-10 mg of polyvinylpyrrolidone, about 50-100 mg of microcrystalline cellulose, about 1-10 mg of croscarmellose sodium, about 0.1-5 mg of magnesium stearate, and a coating material; and the plurality of second particulates comprises about 10-50 mg of antiemetic or a pharmaceutically acceptable salt thereof, about 10-50 mg of microcrystalline cellulose, about 0.1-5 mg of croscarmellose sodium and a coating material. In some instances, the plurality of first particulates comprises about 90 mg of sumatriptan or a therapeutically equivalent amount of pharmaceutically acceptable salt thereof, about 4 mg of polyvinylpyrrolidone, about 69 mg of microcrystalline cellulose, about 4 mg of croscarmellose sodium, about 1 mg of magnesium stearate and a coating material, wherein the coating material comprises polyvinyl alcohol; and the plurality of second particulates comprises about 22 mg of promethazine or a therapeutically equivalent amount of pharmaceutically acceptable salt thereof, about 24 mg of microcrystalline cellulose, about 1 mg of croscarmellose sodium; and a coating material, wherein the coating material comprises polyvinyl alcohol. In some instances, the plurality of first particulates comprises from about 40% to about 80% by weight of the 5HT_{1B} receptor agonist or a pharmaceutically acceptable salt thereof, from about 0.5% it about 5% by weight of polyvinylpyrrolidone, from about 20% to about 60% by weight of microcrystalline cellulose, from about 0.5% to about 5% by weight of croscarmellose sodium, from about 0.1% to about 5% by weight of magnesium stearate and a coating material; and the plurality of second particulates comprises from about 30% to about 70% by weight of the antiemetic or a pharmaceutically acceptable salt thereof, from about 20% to about 70% by weight of microcrystalline cellulose, from about 0.5% to about 5% by weight of croscarmellose sodium and a coating material. In some instances, the plurality of first particulates comprises about 60.5% by weight of sumatriptan succinate, about 2% by weight of polyvinylpyrrolidone, about 35% by weight of microcrystalline cellulose, about 2% by weight of croscarmellose sodium, about 0.5% by weight of magnesium stearate and a coating material, wherein the coating material comprises polyvinyl alcohol; and the plurality of second particulates comprises about 50% by weight of promethazine hydrochloride, about 48% by weight of microcrystalline cellulose, about 2% by weight of croscarmellose sodium, and a coating material, wherein the coating material comprises polyvinyl alcohol. In some instances, the first particulates comprise a coating material. In some instances, the coating material is applied to the plurality of the first particulates at a weight gain of about 2%. In some instances, the second particulates comprise a coating material. In some instances, the coating material is applied to the plurality of the second particulates at a weight gain of about 2%. In some instances, the first particulates and the second particulates comprise the same coating material. In some instances, the coating material comprises polyvinyl alcohol, cellulose acetate phthalate, polyvinyl acetate phthalate, methacrylic acid copolymer, cellulose acetate trimellitate, hydroxypropyl methylcellulose phthalate, hydroxypropyl methylcellulose, hydroxypropyl methyl cellulose acetate succinate, shellac, sodium alginate or zein. In some instances, the coating material comprises polyvinyl alcohol. In some instances, the coating material is polyvinyl alcohol. In some instances, wherein a diameter of each of the first particulates is of from about 595 microns to about 1190 microns. In some instances, a diameter of each of the second particulates is of from about 595 microns to about 1190 microns. In some instances, a diameter of each of the first particulates is of from about 595 microns to about 1190 microns, and a diameter of each of the second particulates is of from about 595 microns to about 1190 microns. In some instances, pharmaceutically acceptable salt of the 5HT_{1B} receptor agonist comprises triptan succinate and the triptan base is present in an amount of about 90 mg. In some instances, the pharmaceutically acceptable salt of the 5HT_{1B} receptor agonist comprises triptan succinate and the triptan base is present in an amount of about 100 mg. In some instances, the pharmaceutically acceptable salt of the 5HT_{1B} receptor agonist comprises sumatriptan succinate and the sumatriptan base is present in an amount of about 90 mg. In some instances, the pharmaceutically acceptable salt of the 5HT_{1B} receptor agonist comprises sumatriptan succinate and the sumatriptan base is present in an amount of about 100 mg. In some instances, the pharmaceutically acceptable salt of the antiemetic comprises promethazine hydrochloride and the promethazine hydrochloride is present in an amount of about 25 mg. In some instances, the pharmaceutical composition is in an oral dosage form. In some instances, that the oral dosage form comprises a capsule. In some instances, the pharmaceutical composition is housed within a container. In some instances, the container is a bottle or pill blister. In some aspects, a pharmaceutical composition disclosed herein for use in treatment of a headache in a subject in need thereof. In some instances, the pharmaceutical composition is for use in treatment of a headache wherein the treatment is acute. In some instances, the pharmaceutical composition is for use in treatment of a headache wherein the treatment is prophylactic. In some instances, the pharmaceutical composition is for use in treatment of a migraine headache. In some instances, the pharmaceutical composition is for use in treatment of an acute migraine headache. In some instances, the pharmaceutical composition is for use in treatment of a chronic migraine headache. In some instances, the pharmaceutical composition is for use in treatment of a migraine headache with or without an aura. In some instances, the pharmaceutical composition is for use in treatment of a cluster headache. In some instances, the pharmaceutical composition is for use in treatment of nausea or vomiting. In some instances, the pharmaceutical composition is for use in treatment of nausea associated with a headache or vomiting associated with a headache. In some instances, the pharmaceutical composition is for use in treatment headache and vomiting associated with a headache. In some aspects, a pharmaceutical composition disclosed herein for use in treatment of a photophobia in a subject in need thereof. In some instances, the pharmaceutical composition is for use in treatment of a photophobia wherein the treatment is acute. In some instances, the pharmaceutical composition is for use in treatment of a photophobia wherein the treatment is prophylactic. In some instances, the pharmaceutical composition is for use in treatment of a light sensitivity. In some instances, the pharmaceutical composition is for use in treatment of nausea or vomiting. In some instances, the pharmaceutical composition is for use in treatment of nausea associated with a headache or vomiting associated with a headache. In some instances, the pharmaceutical composition is for use in treatment headache and vomiting associated with a headache. In some instances, the triptan or a pharmaceutically acceptable salt thereof disclosed herein comprises sumatriptan, almotriptan, frovatriptan, eletriptan, rizatriptan, naratriptan, or a pharmaceutically acceptable salt thereof. In some instances, the antiemetic or a pharmaceutically acceptable salt thereof comprises promethazine, ondansetron, aprepitant, dronabinol, perphenazine, palonosetron, trimethyobenzamide, metoclopromide, domperidone, prochlorperazine, chlorpromazine, trimethobenzamide, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, or a pharmaceutically acceptable salt thereof.

In some aspects, a shelf-stable form of a pharmaceutical composition is provided, the pharmaceutical composition comprising a plurality of first particulates comprising a 5HT_{1B} receptor agonist or a pharmaceutically acceptable salt thereof, wherein about 90% to about 100% of the 5HT_{1B} receptor agonist or a pharmaceutically acceptable salt thereof is stable for at least 30 days as measured by HPLC, and a plurality of second particulates comprising an antiemetic or a pharmaceutically acceptable salt thereof, wherein about 90% to about 100% of the antiemetic or a pharmaceutically acceptable salt thereof is stable for at least 30 days as measured by HPLC. In some instances, about 90% to about 100% of the 5HT_{1B} receptor agonist or a pharmaceutically acceptable salt thereof is stable for at least 90 days. In some instances, about 95% of the 5HT_{1B} receptor agonist or a pharmaceutically acceptable salt thereof is stable for at least 30 days. In some instances, about 90% to about 100% of the antiemetic or the pharmaceutically acceptable salt thereof is stable for at least 90 days. In some instances, about 100% of the antiemetic or the pharmaceutically acceptable salt thereof is stable for at least 30 days. In some instances, the 5HT_{1B} receptor agonist or a pharmaceutically acceptable salt thereof comprises a triptan or a pharmaceutically acceptable salt thereof. In some instances, the triptan or a pharmaceutically acceptable salt thereof comprises sumatriptan or a pharmaceutically acceptable salt thereof. In some instances, the sumatriptan is present in an amount of about 25 mg to about 100 mg. In some instances, the sumatriptan is present in an amount of about 90 mg. In some instances, the pharmaceutically acceptable salt of sumatriptan comprises sumatriptan succinate. In some instances, the sumatriptan succinate is present in an amount of from about 35 mg to about 140 mg. In some instances, the sumatriptan succinate is present in an amount of about 126 mg. In some instances, the pharmaceutically acceptable salt of sumatriptan is present in an amount therapeutically equivalent to about 90 mg of sumatriptan. In some instances, the antiemetic or a pharmaceutically acceptable salt thereof comprises promethazine or a pharmaceutically acceptable salt thereof. In some instances, the promethazine is present in an amount of about 12.5 mg to about 50 mg. In some instances, the promethazine is present in an amount of about 22 mg. In some instances, the pharmaceutically acceptable salt of promethazine comprises promethazine hydrochloride. In some instances, the promethazine hydrochloride is present in an amount of from about 5 mg to about 50 mg, e.g., about 25 mg. In some instances, the pharmaceutically acceptable salt of promethazine is present in an amount therapeutically equivalent to about 22 mg of promethazine. In some instances, a total weight of the plurality of first particulates is of from about 175 mg to about 300 mg. In some instances, the plurality of first particulates is of from about 200 mg to about 220 mg. In some instances, the total weight of the plurality of first particulates is of from about 208 mg to about 212 mg. In some instances, a total weight of the plurality of second particulates is of from about 30 mg to about 100 mg. In some instances, the total weight of the plurality of second particulates is of from about 45 mg to about 55 mg. In some instances, the total weight of the plurality of second particulates is of about 50 mg or about 51 mg. In some instances, the plurality of first particulates comprises one or more first pharmaceutically acceptable excipients, wherein the one or more first pharmaceutically acceptable excipients comprises a diluent, binder, disintegrant or lubricant. In some instances, the diluent comprises microcrystalline cellulose. In some instances, the binder comprises polyvinylpyrrolidone. In some instances, the disintegrant comprises croscarmellose sodium. In some instances, the lubricant comprises magnesium stearate or talc. In some instances, the plurality of second particulates comprises one or more first pharmaceutically acceptable excipients, wherein the one or more first pharmaceutically acceptable excipients comprises a diluent or a disintegrant. In some instances, the diluent comprises microcrystalline cellulose. In some instances, the disintegrant comprises croscarmellose sodium. In some instances, the plurality of first particulates comprises about 50-150 mg of the 5HT_{1B} receptor agonist or a pharmaceutically acceptable salt thereof, about 1-10 mg of polyvinylpyrrolidone, about 50-100 mg of microcrystalline cellulose, about 1-10 mg of croscarmellose sodium, about 0.1-5 mg of magnesium stearate, and a coating material; and the plurality of second particulates comprises about 10-50 mg of antiemetic or a pharmaceutically acceptable salt thereof, about 10-50 mg of microcrystalline cellulose, about 0.1-5 mg of croscarmellose sodium and a coating material. In some instances, the plurality of first particulates comprises about 90 mg of sumatriptan or a therapeutically equivalent amount of pharmaceutically acceptable salt thereof, about 4 mg of polyvinylpyrrolidone, about 69 mg of microcrystalline cellulose, about 4 mg of croscarmellose sodium, about 1 mg of magnesium stearate and a coating material, wherein the coating material comprises polyvinyl alcohol; and the plurality of second particulates comprises about 22 mg of promethazine or a therapeutically equivalent amount of pharmaceutically acceptable salt thereof, about 24 mg of microcrystalline cellulose, about 1 mg of croscarmellose sodium; and a coating material, wherein the coating material comprises polyvinyl alcohol. In some instances, the plurality of first particulates comprises from about 40% to about 80% by weight of the 5HT_{1B} receptor agonist or a pharmaceutically acceptable salt thereof, from about 0.5% it about 5% by weight of polyvinylpyrrolidone, from about 20% to about 60% by weight of microcrystalline cellulose, from about 0.5% to about 5% by weight of croscarmellose sodium, from about 0.1% to about 5% by weight of magnesium stearate and a coating material; and the plurality of second particulates comprises from about 30% to about 70% by weight of the antiemetic or a pharmaceutically acceptable salt thereof, from about 20% to about 70% by weight of microcrystalline cellulose, from about 0.5% to about 5% by weight of croscarmellose sodium and a coating material. In some instances, the plurality of first particulates comprises about 60.5% by weight of sumatriptan succinate, about 2% by weight of polyvinylpyrrolidone, about 35% by weight of microcrystalline cellulose, about 2% by weight of croscarmellose sodium, about 0.5% by weight of magnesium stearate and a coating material, wherein the coating material comprises polyvinyl alcohol; and the plurality of second particulates comprises about 50% by weight of promethazine hydrochloride, about 48% by weight of microcrystalline cellulose, about 2% by weight of croscarmellose sodium, and a coating material, wherein the coating material comprises polyvinyl alcohol. In some instances, the first particulates comprise a coating material. In some instances, the coating material is applied to the plurality of the first particulates at a weight gain of about 2%. In some instances, the second particulates comprise a coating material. In some instances, the coating material is applied to the plurality of the second particulates at a weight gain of about 2%. In some instances, the first particulates and the second particulates comprise the same coating material. In some instances, the coating material comprises polyvinyl alcohol, cellulose acetate phthalate, polyvinyl acetate phthalate, methacrylic acid copolymer, cellulose acetate trimellitate, hydroxypropyl methylcellulose phthalate, hydroxypropyl methylcellulose, hydroxypropyl methyl cellulose acetate succinate, shellac, sodium alginate or zein. In some instances, the coating material comprises polyvinyl alcohol. In some instances, the coating material is polyvinyl alcohol. In some instances, a weight ratio of the plurality of first particulates to the plurality of second particulates is of from about 3:1 to about 5:1. In some instances, the weight ratio of the 5HT_{1B} receptor agonist or a pharmaceutically acceptable salt thereof to the antiemetic or a pharmaceutically acceptable salt thereof is of from about 1:2 to about 15:1. In some instances, at least about 80% of both the 5HT_{1B} receptor agonist or a pharmaceutically acceptable salt thereof and the antiemetic are released within about 15 minutes as measured by contact of the pharmaceutical composition with dissolution fluid in a USP Apparatus 1 (Basket) rotating at 100 rpm. In some instances, the antiemetic or a pharmaceutically acceptable salt thereof has a slower release rate than the release rate of the 5HT_{1B} receptor agonist or a pharmaceutically acceptable salt thereof. In some instances, a diameter of each of the first particulates is of from about 595 microns to about 1190 microns. In some instances, a diameter of each of the second particulates is of from about 595 microns to about 1190 microns. In some instances, a diameter of each of the first particulates is of from about 595 microns to about 1190 microns, and a diameter of each of the second particulates is of from about 595 microns to about 1190 microns. In some instances, the pharmaceutically acceptable salt of the 5HT_{1B} receptor agonist comprises triptan succinate and the triptan base is present in an amount of about 90 mg. In some instances, the pharmaceutically acceptable salt of the 5HT_{1B} receptor agonist comprises triptan succinate and the triptan base is present in an amount of about 100 mg. In some instances, the pharmaceutically acceptable salt of the 5HT_{1B} receptor agonist comprises sumatriptan succinate and the sumatriptan base is present in an amount of about 90 mg. In some instances, the pharmaceutically acceptable salt of the 5HT_{1B} receptor agonist comprises sumatriptan succinate and the sumatriptan base is present in an amount of about 100 mg. In some instances, the pharmaceutically acceptable salt of the antiemetic comprises promethazine hydrochloride and the promethazine hydrochloride is present in an amount of about 25 mg. In some instances, the pharmaceutical composition is in an oral dosage form. In some instances, that the oral dosage form comprises or is a capsule. In some instances, the triptan or a pharmaceutically acceptable salt thereof disclosed herein comprises sumatriptan, almotriptan, frovatriptan, eletriptan, rizatriptan, naratriptan, or a pharmaceutically acceptable salt thereof. In some instances, the antiemetic or a pharmaceutically acceptable salt thereof comprises promethazine, ondansetron, aprepitant, dronabinol, perphenazine, palonosetron, trimethyobenzamide, metoclopromide, domperidone, prochlorperazine, chlorpromazine, trimethobenzamide, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, or a pharmaceutically acceptable salt thereof.

In some aspects, a pharmaceutical composition disclosed herein is for use in treatment of a headache in a subject in need thereof. In some instances, the pharmaceutical composition is for use in treatment of a headache wherein the treatment is acute. In some instances, the pharmaceutical composition is for use in treatment of a headache wherein the treatment is prophylactic. In some instances, the pharmaceutical composition is for use in treatment of a migraine headache. In some instances, the pharmaceutical composition is for use in treatment of an acute migraine headache. In some instances, the pharmaceutical composition is for use in treatment of a chronic migraine headache. In some instances, the pharmaceutical composition is for use in treatment of a migraine headache with or without an aura. In some instances, the pharmaceutical composition is for use in treatment of a cluster headache. In some instances, the pharmaceutical composition is for use in treatment of nausea or vomiting. In some instances, the pharmaceutical composition is for use in treatment of nausea associated with a headache or vomiting associated with a headache. In some instances, the pharmaceutical composition is for use in treatment headache and vomiting associated with a headache. In some aspects, a pharmaceutical composition disclosed herein for use in treatment of a photophobia in a subject in need thereof. In some instances, the pharmaceutical composition is for use in treatment of a photophobia wherein the treatment is acute. In some instances, the pharmaceutical composition is for use in treatment of a photophobia wherein the treatment is prophylactic. In some instances, the pharmaceutical composition is for use in treatment of a light sensitivity. In some instances, the pharmaceutical composition is for use in treatment of nausea or vomiting. In some instances, the pharmaceutical composition is for use in treatment of nausea associated with a headache or vomiting associated with a headache. In some instances, the pharmaceutical composition is for use in treatment headache and vomiting associated with a headache. In some instances, the pharmaceutical composition is housed within a container. In some instances, the container is a bottle or pill blister. In some instances, the pharmaceutical composition comprises a plurality of first particulates comprising a 5HT_{1B} receptor agonist or a pharmaceutically acceptable salt thereof, and a plurality of second particulates comprising an antiemetic or a pharmaceutically acceptable salt thereof. In some instances, the triptan or a pharmaceutically acceptable salt thereof disclosed herein comprises sumatriptan, almotriptan, frovatriptan, eletriptan, rizatriptan, naratriptan, or a pharmaceutically acceptable salt thereof. In some instances, the antiemetic or a pharmaceutically acceptable salt thereof comprises promethazine, ondansetron, aprepitant, dronabinol, perphenazine, palonosetron, trimethyobenzamide, metoclopromide, domperidone, prochlorperazine, chlorpromazine, trimethobenzamide, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, or a pharmaceutically acceptable salt thereof. In some embodiments, a pharmaceutical composition disclosed herein is administered to a subject at about every 12 to about 24 hours, about every 12 hours, or about every 24 hours. In some embodiments, a pharmaceutical composition disclosed herein is administered to a subject at about every 8 to about every 12 hours. In some embodiments, a pharmaceutical composition disclosed herein is administered once, twice or three times daily. In some embodiments, a pharmaceutical composition described herein is administered no more than twice daily. In some embodiments, a second dose of a pharmaceutical composition disclosed herein is administered after response to a first dose in the subject. In some embodiments, doses after a first dose of a pharmaceutical composition disclosed herein are separated by at least 2 hours. In some embodiments, the maximum dose of a pharmaceutical composition disclosed herein over a 24 hour period does not exceed 200 mg. In some embodiments, a maximum single dose of a pharmaceutical composition disclosed herein dose does not exceed 50 mg in a subject with mild to moderate hepatic impairment. In some embodiments, a pharmaceutical composition disclosed herein comprising sumatriptan succinate and promethazine hydrochloride is administered to a subject at about every 12 to about 24 hours, about every 12 hours, or about every 24 hours. In some embodiments, a pharmaceutical composition disclosed herein comprising sumatriptan succinate and promethazine hydrochloride is administered to a subject at about every 8 to about every 12 hours. In some embodiments, a pharmaceutical composition disclosed herein comprising sumatriptan succinate and promethazine hydrochloride is administered once, twice or three times daily. In some embodiments, a pharmaceutical composition disclosed herein comprising sumatriptan succinate and promethazine hydrochloride is administered no more than twice daily. In some embodiments, a second dose of a pharmaceutical composition disclosed herein comprising sumatriptan succinate and promethazine hydrochloride is administered after response to a first dose in the subject. In some embodiments, doses after a first dose of a pharmaceutical composition disclosed herein are separated by at least 2 hours. In some embodiments, the maximum dose of a pharmaceutical composition disclosed herein comprising sumatriptan succinate and promethazine hydrochloride over a 24 hour period does not exceed 200 mg. In some embodiments, a maximum single dose of a pharmaceutical composition disclosed herein comprising sumatriptan succinate and promethazine hydrochloride does not exceed 50 mg in a subject with mild to moderate hepatic impairment. In some embodiments, the frequency of dosing is determined or assessed by a professional assessing the subject, the severity of the condition and expected duration of therapy.

In some aspects, a method is provided for treating a headache in a subject in need thereof, comprising administering to the subject a pharmaceutical composition disclosed herein. In some instances, the treatment of the headache is acute or prophylactic. In some instances, the treatment of the headache is a migraine headache. In some instances, the headache is an acute migraine headache or a chronic migraine headache. In some instances, the headache is a migraine headache with or without an aura. In some instances, the headache is a cluster headache. In some aspects, a method is provided for treating a photophobia in a subject in need thereof, comprising administering to the subject a pharmaceutical composition disclosed herein. In some instances, the treatment of the photophobia is acute or prophylactic. In some instances, the pharmaceutical composition is used for treatment of a light sensitivity. In some instances, the pharmaceutical composition treats nausea or vomiting. In some instances, the pharmaceutical composition treats nausea associated with a headache or vomiting associated with a headache. In some instances, the pharmaceutical composition treats nausea associated with a headache and vomiting associated with a headache. In some instances, the administering delivers about 25 mg to about 100 mg of sumatriptan. In some instances, the administering delivers about 50 mg to about 75 mg of sumatriptan. In some instances, the administering delivers about 50 mg to about 100 mg of sumatriptan. In some instances, the administering is one, two, or three times daily. In some instances, the administering is about every 8 to about every 12 hours. In some instances, a second dose of the pharmaceutical composition is administered after response to a first dose in the subject. In some instances, doses after a first dose of the pharmaceutical composition are separated by at least 2 hours. In some instances, a maximum dose of the pharmaceutical composition over a 24 hour period does not exceed 200 mg. In some instances, a maximum single dose of the pharmaceutical composition does not exceed 50 mg in a subject with mild to moderate hepatic impairment. In some instances, the pharmaceutical composition comprises a plurality of first particulates comprising a 5HT_{1B} receptor agonist or a pharmaceutically acceptable salt thereof, and a plurality of second particulates comprising an antiemetic or a pharmaceutically acceptable salt thereof. In some instances, the triptan or a pharmaceutically acceptable salt thereof disclosed herein comprises sumatriptan, almotriptan, frovatriptan, eletriptan, rizatriptan, naratriptan, or a pharmaceutically acceptable salt thereof. In some instances, the antiemetic or a pharmaceutically acceptable salt thereof comprises promethazine, ondansetron, aprepitant, dronabinol, perphenazine, palonosetron, trimethyobenzamide, metoclopromide, domperidone, prochlorperazine, chlorpromazine, trimethobenzamide, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, or a pharmaceutically acceptable salt thereof.

In some aspects, a capsule is provided, the capsule comprising a capsule layer; a plurality of first particulates, wherein each of the first particulates comprises a first active pharmaceutical ingredient, the plurality of the first particulates is surrounded by the capsule layer, and each of the first particulates is in the shape of a bead, spherule, or pellet; and a plurality of second particulates, wherein each of the second particulates comprises a second active pharmaceutical ingredient, the plurality of the second particulates is surrounded by the capsule layer, and each of the second particulates is in the shape of a bead, spherule, or pellet, and a weight ratio of the plurality of the first particulates to the plurality of the second particulates is of from about 3:1 to about 5:1. In some instances, a weight ratio of the first active pharmaceutical ingredient to the second active pharmaceutical ingredient is of from about 1:2 to about 15:1. In some instances, the weight ratio of the first active pharmaceutical ingredient to the second active pharmaceutical ingredient is about 5:1. In some instances, the weight ratio of the plurality of the first particulates to the plurality of the second particulates is about 4:1. In some instances, a weight ratio of the first active pharmaceutical ingredient to a total weight of the plurality of the first particulates is of from about 2:5 to about 7:10. In some instances, the weight ratio of the second active pharmaceutical ingredient to a total weight of the plurality of the second particulates is of from about 2:5 to about 3:5. In some instances, the plurality of the first particulates comprises one or more first pharmaceutically acceptable excipients, and a weight ratio of a total amount of the first active pharmaceutical ingredient to a total amount of the one or more first pharmaceutically acceptable excipients is about 3:2. In some instances, the one or more first pharmaceutically acceptable excipients comprises a diluent, binder, disintegrant or lubricant. In some instances, the diluent is present in an amount of about 35% by weight of the plurality of the first particulates. In some instances, the binder is present in an amount of about 0.5% to about 5% by weight of the plurality of the first particulates. In some instances, the disintegrant is present in an amount of about 2% by weight of the plurality of the first particulates. In some instances, the lubricant is present in an amount of about 0.5% by weight of the plurality of the first particulates. In some instances, the plurality of the second particulates comprises one or more second pharmaceutically acceptable excipients, and a weight ratio of a total amount of the second active pharmaceutical ingredient to a total amount of the one or more second pharmaceutically acceptable excipients is about 1:1. In some instances, the one or more second pharmaceutically acceptable excipients comprises a diluent or a disintegrant. In some instances, the diluent is present in an amount of from about 20% to about 90% by weight of the plurality of the second particulates. In some instances, the disintegrant is present in an amount of from about 0.5% to about 2% by weight of the plurality of the second particulates. In some instances, a diameter of each of the first particulates is of from about 595 microns to about 1190 microns. In some instances, the diameter of each of the first particulates is of from about 595 microns to about 707 microns, from about 707 microns to about 841 microns, from about 841 microns to about 1000 microns, or from about 1000 microns to about 1190 microns. In some instances, a diameter of each of the second particulates is of from about 595 microns to about 1190 microns. In some instances, the diameter of each of the second particulates is of from about 595 microns to about 707 microns, from about 707 microns to about 841 microns, from about 841 microns to about 1000 microns, or from about 1000 microns to about 1190 microns. In some instances, each of the first particulates and each of the second particulates have a diameter of from about 595 microns to about 1190 microns. In some instances, a total weight of the plurality of the first particulates is of from about 175 mg to about 300 mg. In some instances, the total weight of the plurality of the first particulates is of from about 208 mg to about 212 mg. In some instances, a total weight of the plurality of the second particulates is of from about 30 mg to about 100 mg. In some instances, the total weight of the plurality of the second particulates is of from about 45 mg to about 55 mg. In some instances, the first active pharmaceutical ingredient is present in an amount of from about 25 mg to about 150 mg. In some instances, the first active pharmaceutical ingredient is present in an amount of about 90 mg or about 126 mg. In some instances, a total amount of the first active pharmaceutical ingredient is present in an amount of from about 50% to about 70% by weight of the plurality of the first particulates. In some instances, the total amount of the first active pharmaceutical ingredient is present in an amount of about 61% by weight of the plurality of the first particulates. In some instances, the first active pharmaceutical ingredient comprises sumatriptan or a pharmaceutically acceptable salt thereof. In some instances, the pharmaceutically acceptable salt of the sumatriptan comprises sumatriptan succinate. In some instances, the pharmaceutically acceptable salt of the sumatriptan is sumatriptan succinate. In some instances, a total amount of the pharmaceutically acceptable salt of the sumatriptan is present in an amount therapeutically equivalent to about 90 mg of sumatriptan. In some instances, the second active pharmaceutical ingredient is present in an amount of from about 40% to about 60% by weight of the plurality of the second particulates. In some instances, the second active pharmaceutical ingredient is present in an amount of about 50% by weight of the plurality of the second particulates. In some instances, the second active pharmaceutical ingredient is present in an amount of from about 12.5 mg to about 50 mg. In some instances, the second active pharmaceutical ingredient is present in an amount of about 22 mg or about 25 mg. In some instances, the second active pharmaceutical ingredient comprises promethazine or a pharmaceutically acceptable salt thereof. In some instances, the pharmaceutically acceptable salt of the promethazine comprises promethazine hydrochloride. In some instances, the pharmaceutically acceptable salt of the promethazine is promethazine hydrochloride. In some instances, a total amount of the pharmaceutically acceptable salt of the promethazine is present in an amount therapeutically equivalent to about 22 mg of promethazine. In some instances, the capsule has a net weight of from about 90 mg to about 102 mg. In some instances, the capsule has a net weight of about 96 mg. In some instances, the capsule has a volume of from about 0.6 ml to about 0.8 ml. In some instances, the capsule has a volume of about 0.7 ml. In some instances, a body of the capsule is of from about 17 mm to about 20 mm long. In some instances, a body of the capsule is about 18 mm long. In some instances, a cap of the capsule is of from about 10 mm to 12 mm long. In some instances, a cap of the capsule is about 11 mm long. In some instances, a body of the capsule has an external diameter of from about 6 mm to about 8 mm. In some instances, a body of the capsule has an external diameter of about 7 mm. In some instances, a cap of the capsule has an external diameter of from about 7 mm to about 9 mm. In some instances, a cap of the capsule has an external diameter of about 8 mm. In some instances, an overall closed length of the capsule is of from about 20 mm to 24 mm. In some instances, an overall closed length of the capsule is about 22 mm. In some instances, the capsule has a capacity of about 400-800 mg and a powder density of about 0.6 to about 1.2 g/ml. In some instances, each of the first particulates and each the second particulates are the same shape. In some instances, the first particulates comprise a coating material. In some instances, the coating material is applied to the plurality of the first particulates at a weight gain of about 2%. In some instances, the second particulates comprise a coating material. In some instances, the coating material is applied to the plurality of the second particulates at a weight gain of about 2%. In some instances, the first particulates and the second particulates comprise the same coating material. In some instances, the coating material comprises polyvinyl alcohol, cellulose acetate phthalate, polyvinyl acetate phthalate, methacrylic acid copolymer, cellulose acetate trimellitate, hydroxypropyl methylcellulose, hydroxypropyl methylcellulose phthalate, hydroxy propyl methyl cellulose acetate succinate, shellac, sodium alginate or zein. In some instances, the coating material comprises polyvinyl alcohol. In some instances, the coating material is polyvinyl alcohol. In some instances, the capsule is housed within a container. In some instances, the container is a bottle or pill blister.

### BRIEF DESCRIPTION OF THE DRAWINGS

FIG. 1 is an HPLC chromatograph of a dissolution fluid disclosed herein.

FIGS. 2A and 2B are HPLC chromatographs of standards for sumatriptan and promethazine displayed in full view (FIG. 2A) and expanded view (FIG. 2B).

FIGS. 3A and 3B are HPLC chromatographs of a test sample showing dissolution measurements displayed in full view (FIG. 3A) and expanded view (FIG. 3B).

FIG. 4 is a line graph showing dissolution rates for sumatriptan and promethazine in Formulation I following contact with a dissolution fluid.

FIG. 5 is a line graph showing dissolution rates for sumatriptan and promethazine in Formulation II following contact with a dissolution fluid.

FIG. 6 illustrates an exemplary capsule, unfilled (left, in side and bottom view) or filled (right) with particulates.

FIG. 7 illustrates another exemplary capsule, unfilled (left, in top, side and bottom view) or filled (right) with particulates.

### INCORPORATION BY REFERENCE

All publications, patents, and patent applications disclosed herein are incorporated by reference to the same extent as if each individual publication, patent, or patent application was specifically and individually indicated to be incorporated by reference. In the event of a conflict between a term disclosed herein and a term in an incorporated reference, the term herein controls.

### DETAILED DESCRIPTION

This disclosure is generally directed to compositions comprising multiple pharmaceutically active agents for the alleviation, abatement or elimination of one or more conditions in a subject in need thereof, as further described herein below.

A “therapeutically effective amount” when used in connection with a pharmaceutical composition described herein is an amount of one or more pharmaceutically active agent(s) sufficient to produce a therapeutic result in a subject in need thereof. For example, a therapeutic result includes, but is not limited to, treating pain, migraine headache, nausea, vomiting, photophobia, phonophobia or osmophobia by a subject.

“Therapeutically equivalent” when used in connection with a pharmaceutical composition described herein refers to an amount or quantity of a pharmaceutically acceptable salt of a pharmaceutically active agent that is equivalent to the therapeutically effective amount of the free base of the pharmaceutically active agent.

In some embodiments, therapeutic results produced herein include reducing or eliminating one or more adverse effects associated with one or more pharmaceutically active agents disclosed herein. In some embodiments, adverse effects reduced or eliminated include, but are not limited to, nausea or vomiting.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

In some aspects, a pharmaceutical composition disclosed herein comprises a therapeutically effective amount of a first pharmaceutically active agent; a second pharmaceutically active agent capable of reducing or eliminating adverse effects associated with the first pharmaceutically active agent; and a pharmaceutically acceptable carrier or vehicle. In some embodiments, a pharmaceutical composition disclosed herein comprises a therapeutically effective amount of a triptan; an antiemetic; and a pharmaceutically acceptable carrier or vehicle. In some embodiments, a pharmaceutical composition disclosed herein comprises a therapeutically effective amount of sumatriptan or a pharmaceutically acceptable salt thereof; promethazine or a pharmaceutically acceptable salt thereof; and a pharmaceutically acceptable carrier or vehicle. In some embodiments, a pharmaceutical composition disclosed herein comprises a therapeutically effective amount of a triptan; an antiemetic; a polymer; and a pharmaceutically acceptable carrier or vehicle. In some embodiments, a pharmaceutical composition disclosed herein comprises a therapeutically effective amount of a triptan; an antiemetic; a vinyl polymer; and a pharmaceutically acceptable carrier or vehicle. In some embodiments, a pharmaceutical composition disclosed herein comprises a therapeutically effective amount of sumatriptan or a pharmaceutically acceptable salt thereof; promethazine or a pharmaceutically acceptable salt thereof; polyvinylpyrrolidone; and a pharmaceutically acceptable carrier or vehicle. In some embodiments, a pharmaceutical composition disclosed herein comprises a therapeutically effective amount of a triptan; an antiemetic; a vinyl copolymer; and a pharmaceutically acceptable carrier or vehicle.

In some embodiments, a pharmaceutical composition disclosed herein comprises a plurality of first particulates comprising a therapeutically effective amount of a first pharmaceutically active agent and one or more first pharmaceutically acceptable excipients; and a plurality of second particulates comprising a therapeutically effective amount of a second pharmaceutically active agent and one or more second pharmaceutically acceptable excipients; wherein the one or more first pharmaceutically acceptable excipients comprises a polymer.

Pharmaceutically active agents disclosed herein are capable of use in a pharmaceutical composition as described herein. In some embodiments, a pharmaceutically active agent is a triptan, an antiemetic, or a pharmaceutically acceptable salt thereof.

Triptans

In some embodiments, a pharmaceutical composition disclosed herein comprises a 5HT_{1B} receptor agonist. Exemplary 5HT_{1B} receptor agonists include, without limitation, ergotamine and triptan family compounds. Exemplary triptans include, without limitation, sumatriptan, almotriptan, frovatriptan, eletriptan, rizatriptan, and naratriptan. In some embodiments, a pharmaceutical composition disclosed herein comprises a triptan or triptan analog. Triptan analogs are generally a family of tryptamine based drugs used for the treatment of migraines and headaches. Their action is attributed to their binding to serotonin receptors in nerve ending and in cranial blood vessels (causing their constriction) and subsequent inhibition of pro-inflammatory neuropeptide release. Exemplary triptans include, sumatriptan, almotriptan, forvatriptan, rizatriptan, zolmitriptan, eletriptan, and naratriptan, and pharmaceutically acceptable salts thereof. In some embodiments, triptan is used in a pharmaceutical composition disclosed herein is a free base or in the form of pharmaceutically acceptable salt thereof, for example, in the form of succinate. In some embodiments, the triptan is sumatriptan or a pharmaceutically acceptable salt thereof. In some embodiments, the triptan is a triptan or pharmaceutically acceptable salt thereof listed in Table 16. In some embodiments, a pharmaceutical composition disclosed herein comprises one or more pharmaceutically active agents provided in Table 16, or a pharmaceutically acceptable salt thereof.

Antiemetics

In some embodiments, pharmaceutical compositions disclosed herein comprise one or more antiemetics. Exemplary antiemetics include, aprepitant, dronabinol, perphenazine, palonosetron, trimethyobenzamide, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, and pharmaceutically acceptable salts thereof. Antiemetics also include H1 agonists, H1 antagonists, H2 agonists, H2 antagonists, H3 agonists, H3 antagonists, H4 agonists, and H4 antagonists. Examples of such agonists and antagonists include, but are not limited to, 2-(m-fluoropheny)-histamine, azelastine, buclizine, carbinoxamine, cetrizine, clemastine, cyproheptadine, desloratidine, dimenhydrinate, diphenhydramine, emedastine, fexofenadine, hydroxyzine, ketotifen, levocabastine, olopatadine, phenindamine, promethazine, chlorpheniramine, scopolamine, mepyramine, terfenadine, astemizole, triprolidine, dimaprit, impromidine, amthamine, cimetidine, ranitidine, nizatidine, famotidine, R-alpha-methylhistamine, imetit, immepip, thioperamide, iodophenpropit, clobenpropit, clozapine, and a pharmaceutically acceptable salt thereof. In some embodiments, the second pharmaceutically active agent is an antiemetic. In some embodiments, the antiemetic is promethazine or a pharmaceutically acceptable salt thereof. In some embodiments, the antiemetic is an antiemetic or pharmaceutically acceptable salt thereof listed in Table 16. In some embodiments, a pharmaceutical composition disclosed herein comprises one or more pharmaceutically active agents provided in Table 16, or a pharmaceutically acceptable salt thereof.

Pharmaceutically Acceptable Salts

In some embodiments, an agent used in a composition disclosed herein is the form of a free base, pharmaceutically acceptable salt, prodrug, analog or complex. In some instances, a pharmaceutically active agent comprises the form of a pharmaceutically acceptable salt. In various embodiments, with respect to a pharmaceutically active agent in a composition, a pharmaceutically acceptable salt includes, but is not limited to, metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like.

In some embodiments, pharmaceutically acceptable salts include bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafluorobutyrate), bis(pentafluoropropionate), bis(pyridine carboxylate), bis(trifluoroacetate), chlorhydrate, and sulfate pentahydrate. In some embodiments, an agent is promethazine, a pharmaceutically acceptable salt or its thiosemicarbazone, p-nitrophenylhydrazone, o-methyloxime, semicarbazone, or bis(methylcarbamate). Other representative pharmaceutically acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A hydrate is another example of a pharmaceutically acceptable salt. In some embodiments, the second pharmaceutically active agent is capable of reducing or eliminating an adverse effect of the first pharmaceutically active agent.

Pharmaceutically Acceptable Excipients

In some aspects, a pharmaceutical composition disclosed herein comprises one or more pharmaceutically acceptable excipients. Exemplary pharmaceutically acceptable excipients for the purposes of pharmaceutical compositions disclosed herein include, but are not limited to, binders, disintegrants, superdisintegrants, lubricants, diluents, fillers, flavors, glidants, sorbents, solubilizers, chelating agents, emulsifiers, thickening agents, dispersants, stabilizers, suspending agents, adsorbents, granulating agents, preservatives, buffers, coloring agents and sweeteners or combinations thereof. Examples of binders include microcrystalline cellulose, hydroxypropyl methylcellulose, carboxyvinyl polymer, polyvinylpyrrolidone, polyvinylpolypyrrolidone, carboxymethylcellulose calcium, carboxymethylcellulose sodium, ceratonia, chitosan, cottonseed oil, dextrates, dextrin, ethylcellulose, gelatin, glucose, glyceryl behenate, galactomannan polysaccharide, hydroxyethyl cellulose, hydroxyethylmethyl cellulose, hydroxypropyl cellulose, hypromellose, inulin, lactose, magnesium aluminum silicate, maltodextrin, methylcellulose, poloxamer, polycarbophil, polydextrose, polyethylene glycol, polyethylene oxide, polymethacrylates, sodium alginate, sorbitol, starch, sucrose, sunflower oil, vegetable oil, tocofersolan, zein, or combinations thereof. Examples of disintegrants include croscarmellose sodium, sodium starch glycolate, lactose, magnesium aluminum silicate, methylcellulose, polacrilin potassium, sodium alginate, starch, or combinations thereof. Examples of a lubricant include stearic acid, sodium stearyl fumarate, glyceryl behenate, calcium stearate, glycerin monostearate, glyceryl palmitostearate, magnesium lauryl sulfate, mineral oil, palmitic acid, myristic acid, poloxamer, polyethylene glycol, sodium benzoate, sodium chloride, sodium lauryl sulfate, talc, zinc stearate, potassium benzoate, magnesium stearate or combinations thereof. Examples of diluents include talc, ammonium alginate, calcium carbonate, calcium lactate, calcium phosphate, calcium silicate, calcium sulfate, cellulose, cellulose acetate, corn starch, dextrates, dextrin, dextrose, erythritol, ethylcellulose, fructose, fumaric acid, glyceryl palmitostearate, isomalt, kaolin, lactitol, lactose, magnesium carbonate, magnesium oxide, maltodextrin, maltose, mannitol, microcrystalline cellulose, polydextrose, polymethacrylates, simethicone, sodium alginate, sodium chloride, sorbitol, starch, sucrose, sulfobutylether β-cyclodextrin, tragacanth, trehalose, xylitol, or combinations thereof.

In some embodiments, at least one of the one or more pharmaceutically acceptable excipients is a polymer. In some aspects, a pharmaceutical composition as disclosed herein comprises one or more pharmaceutically acceptable excipients that comprises a polymer and a remaining one or more pharmaceutically acceptable excipients. In some embodiments, the polymer is a vinyl polymer or vinyl copolymer. In some embodiments, the vinyl polymer is polyvinylpyrrolidone or polyvinylpolypyrrolidone.

In some embodiments, a pharmaceutical composition disclosed herein comprises polyvinylpyrrolidone having an average molecular weight of about 10,000 to about 1,000,000 daltons, about 20,000 to about 200,000 daltons, about 30,000 to about 100,000 daltons, about 30,000 to about 50,000 daltons, about 10,000 to about 20,000 daltons, about 20,000 to about 30,000 daltons, 30,000 to about 40,000 daltons, 40,000 to about 50,000 daltons, about 50,000 to about 60,000 daltons, about 60,000 to about 70,000 daltons, about 70,000 to about 80,00 daltons, about 80,000 to about 90,000 daltons, about 90,000 to about 100,000 daltons, about 100,000 to about 200,000 daltons, about 200,000 to about 400,000 daltons, about 400,000 to about 750,000 daltons, about 750,000 to about 1,000,000 daltons.

In some embodiments, a pharmaceutical composition disclosed herein comprises polyvinylpyrrolidone having a K-value of about 12 to about 120, including, but not limited to, one or more of 12, 15, 17, 25, 26, 27, 28, 29, 30, 31, 32, 33, 34, 35, 60, 90, or 120. In some embodiments, pharmaceutical compositions comprise polyvinylpyrrolidone having a K-value selected from a group consisting of: about 12 to about 120, about 12 to about 15, about 15 to about 17, about 17 to about 25, about 25 to about 35, about 25 to about 32, about 24 to about 30, about 29 to about 32, about 30 to about 60, about 60 to about 90, or about 90 to about 120. In some embodiments, the polymer is a vinyl copolymer, such as a polyvinylpyrrolidone copolymer comprising polyvinylpyrrolidone and an additional polymer. In some embodiments, the additional polymer is selected from a group consisting of polyvinyl acetate, vinyl acetate, and polyethylene glycol. In some embodiments, the additional polymer is selected from a group consisting of dimethylaminoethyl methacrylate, styrene, and 1-triacontene. In some embodiments, the vinyl copolymer is a polyvinylpyrrolidone/vinyl acetate, polyvinylpyrrolidone/polyvinyl acetate, polyvinylpyrrolidone/polyethylene glycol, or a vinylpyrrolidone/vinyl acetate copolymer. In some embodiments, the vinyl copolymer is a polyvinylpyrrolidone/dimethylaminoethyl methacrylate, polyvinylpyrrolidone/styrene, or polyvinylpyrrolidone/1-triacontene copolymer. In some embodiments, a pharmaceutical composition disclosed herein comprises a vinyl copolymer having polyvinylpyrrolidone and an additional polymer, wherein the relative ratio by weight of each of polyvinylpyrrolidone to an additional polymer is about (1 to 7):(2 to 9), such as about 1:2, 2:2, 2:3, 2:4, 2:5, 2:6, 2:7, 2:8, 2:9, 3:2, 3:4, 3:5, 3:7, 3:8, 4:2, 4:3, 4:5, 4:6, 4:7, 4:9, 5:2, 5:3, 5:4, 5:6, 5:7, 5:8, 5:9, 6:2, 6:4, 6:5, 6:7, 6:8, 6:9, 7:2, 7:3, 7:4, 7:5, 7:6, 7:8, 7:9. In some embodiments, a pharmaceutical composition disclosed herein comprises a vinyl copolymer having polyvinylpyrrolidone and an additional polymer, wherein the relative ratio by weight of each of polyvinylpyrrolidone to an additional polymer is about (1 to 7):(2 to 9), such as about 2:8 to about 7:3, or about 4:6 to about 7:3. In some embodiments, a pharmaceutical composition disclosed herein comprises a polyvinylpyrrolidone copolymer having a polyvinylpyrrolidone:vinyl acetate ratio of about 60:40. In some embodiments, a pharmaceutical composition disclosed herein comprises a vinyl copolymer that is a vinylpyrrolidone copolymer. In some embodiments, the vinylpyrrolidone copolymer comprises vinylpyrrolidone and vinyl acetate. In some embodiments, a pharmaceutical composition disclosed herein comprises a vinylpyrrolidone copolymer having vinylpyrrolidone and vinyl acetate, wherein the relative ratio by weight of each of polyvinylpyrrolidone:vinyl acetate is about 60 to 40.

### Dosage

In some aspects, a pharmaceutical composition disclosed herein comprises multiple pharmaceutically active agents at the same or different dosages. In some embodiments a pharmaceutically active agent such as triptan varies in dosages as further described herein, and the dosage of a pharmaceutically active agent such as an antiemetic is adjusted according to the particular triptan used. In some embodiments a pharmaceutical composition comprises a triptan or a pharmaceutically acceptable salt thereof that is present at a dose of from about 1.0 mg to about 200 mg, including, but not limited to, about 25 mg to about 100 mg, about 35 mg to about 140 mg, about 70 mg to about 140 mg, about 80 mg to about 135 mg, about 1.0 mg to about 25 mg, about 25 mg to about 50 mg, about 50 mg to about 100 mg, about 100 mg to about 150 mg, about 150 mg to about 200 mg, about 1.0 mg to about 35 mg, about 35 mg to about 70 mg, about 70 mg to about 105 mg, about 105 mg to about 140 mg, about 140 mg to about 175 mg, or about 175 mg to about 200 mg. In some embodiments a pharmaceutical composition comprises a triptan or a pharmaceutically acceptable salt thereof that is present at a dosage of from about 1.0 mg to about 200 mg, including, but not limited to, about 1.0 mg, 1.5 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 9.0 mg, 9.5 mg, 10.0 mg, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100, 105 mg, 110 mg, 115 mg, 120 mg, 120.5 mg, 121 mg, 121.5 mg, 122 mg, 122.5 mg, 123 mg, 123.5 mg, 124 mg, 124.5 mg, 125 mg, 125.5 mg, 126 mg, 126.5 mg, 127 mg, 127.5 mg, 128 mg, 128.5 mg, 129 mg, 129.5 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some embodiments the triptan is sumatriptan or a pharmaceutically acceptable salt thereof. In some embodiments, a pharmaceutical composition comprises a pharmaceutically acceptable salt of triptan in a quantity therapeutically equivalent to triptan dosages disclosed herein. In some embodiments, a pharmaceutical composition comprises a pharmaceutically acceptable salt of sumatriptan in a quantity therapeutically equivalent to 90 mg sumatriptan.

In some embodiments, an amount of sumatriptan or a pharmaceutical acceptable salt thereof (e.g., sumatriptan succinate) present in a pharmaceutical composition disclosed herein is equivalent to about: 4 mg, 6 mg, 10 mg, 25 mg, 50 mg, 85 mg, 90 mg, or 100 mg of free-base sumatriptan. In some embodiments, an amount of sumatriptan succinate present in a pharmaceutical composition disclosed herein is about: 35 mg, 70 mg, 126 mg, or 140 mg. In some embodiments, an amount of free-base sumatriptan present in a pharmaceutical composition disclosed herein is about: 25 mg to 50 mg, 50 mg to 100 mg, or 75 mg to 100 mg.

In some embodiments, a weight ratio of a plurality of first particulates to a plurality of second particulates is of from about 2:1 to about 6:1, or from about 3:1 to about 5:1, respectively, for example about 4:1. In some embodiments, a weight ratio of a first active pharmaceutical ingredient to a total amount of one or more first pharmaceutically acceptable excipients is of from about 1:1 to about 2:1, respectively, for example about 3:2. In some embodiments, a weight ratio of a second active pharmaceutical ingredient to a total amount of one or more second pharmaceutically acceptable excipients is of from about 2:1 to about 1:2, respectively, for example about 1:1. In some embodiments, a weight ratio of a first active pharmaceutical ingredient (e.g., triptan or a pharmaceutically acceptable salt thereof such as sumatriptan succinate) to a second active pharmaceutical ingredient (e.g., antiemetic such as promethazine or a pharmaceutically acceptable salt thereof for example promethazine hydrochloride) is of from about 1:2 to about 15:1, respectively, for example about: 5:1, 1:1, 2:1, 3:1, 4:1, 6:1, 7:1, 8:1, 9:1, 10:1, 11:1, 12:1, 13:1, 14:1. In some embodiments, a weight ratio of a first active pharmaceutical ingredient to a total weight of a plurality of first particulates is about: 40-80%, 45-75%, 50-70%, or 55-65%, for example about 60%. In some embodiments, a weight ratio of a second active pharmaceutical ingredient to a total weight of a plurality of second particulates is about: 30-70%, 35-65%, 40-60%, or 45-55%, for example about 50%.

In some embodiments a pharmaceutical composition disclosed herein comprises an antiemetic or a pharmaceutically acceptable salt thereof that is present at a dose of from about 0.5 mg to about 100 mg, including but not limited to, about 0.5 mg to about 12.5 mg, about 12.5 mg to about 50 mg, about 50 mg to about 75 mg, about 75 mg to about 100 mg, about 0.5 mg to about 15 mg, about 15 mg to about 35 mg, about 35 mg to about 55 mg, about 55 mg to about 75 mg, or about 75 mg to about 95 mg. In some embodiments, a pharmaceutical composition comprises an antiemetic or a pharmaceutically acceptable salt thereof that is present at a dose of from about 0.5 mg to about 100 mg, including but not limited to, about 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, or 100 mg. In some embodiments, the antiemetic is promethazine or a pharmaceutically acceptable salt thereof. In some embodiments, the antiemetic is provided at a dose to prevent or reduce sedation. In some embodiments, a pharmaceutical composition comprises a pharmaceutically acceptable salt of an antiemetic in a quantity therapeutically equivalent to antiemetic dosages disclosed herein. In some embodiments, a pharmaceutical composition comprises a pharmaceutically acceptable salt of promethazine in a quantity therapeutically equivalent to 22 mg promethazine.

In some embodiments, a pharmaceutical composition disclosed herein comprises a triptan and an antiemetic. In some embodiments, the triptan is present at a dose of from about 1.0 mg to about 200 mg, including, but not limited to, about 1.0 mg, 1.5 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 9.0 mg, 9.5 mg, 10.0 mg, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100, 105 mg, 110 mg, 115 mg, 120 mg, 120.5 mg, 121 mg, 121.5 mg, 122 mg, 122.5 mg, 123 mg, 123.5 mg, 124 mg, 124.5 mg, 125 mg, 125.5 mg, 126 mg, 126.5 mg, 127 mg, 127.5 mg, 128 mg, 128.5 mg, 129 mg, 129.5 mg, 130 mg, 135 mg, 140 mg, 145 mg, 1150 mg, 155 mg, 160 mg, 165 mg, 170 mg, 175 mg, 180 mg, 185 mg, 190 mg, 195 mg, or 200 mg. In addition, the antiemetic is present at a dose from about 0.5 mg to about 100 mg, including, but not limited to, 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, or 100 mg. In some embodiments, the triptan is sumatriptan or a pharmaceutically acceptable salt thereof and the antiemetic is promethazine or a pharmaceutically acceptable salt thereof. In some embodiments, a pharmaceutical composition comprises a pharmaceutically acceptable salt of an antiemetic in a quantity therapeutically equivalent to antiemetic dosages disclosed herein. In some embodiments, a pharmaceutical composition comprises a pharmaceutically acceptable salt of promethazine in a quantity therapeutically equivalent to promethazine dosages disclosed herein.

In some embodiments, a pharmaceutical composition disclosed herein comprises sumatriptan, or a pharmaceutically acceptable salt thereof, that is present at a free base dose of from about 10 mg to about 200 mg, including, but not limited to, about 25 mg to about 100 mg, about 35 mg to about 140 mg, about 70 mg to about 140 mg, about 80 mg to about 135 mg, about 10 mg to about 25 mg, about 25 mg to about 50 mg, about 50 mg to about 100 mg, about 100 mg to about 150 mg, about 150 mg to about 200 mg, about 10 mg to about 35 mg, about 35 mg to about 70 mg, about 70 mg to about 105 mg, about 105 mg to about 140 mg, about 140 mg to about 175 mg, or about 175 mg to about 200 mg. In some embodiments a pharmaceutical composition comprises sumatriptan, or a pharmaceutically acceptable salt thereof, that is present at a dose of from about 10 mg to about 200 mg, including, but not limited to, about 10.0 mg, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100, 105 mg, 110 mg, 115 mg, 120 mg, 120.5 mg, 121 mg, 121.5 mg, 122 mg, 122.5 mg, 123 mg, 123.5 mg, 124 mg, 124.5 mg, 125 mg, 125.5 mg, 126 mg, 126.5 mg, 127 mg, 127.5 mg, 128 mg, 128.5 mg, 129 mg, 129.5 mg, 130 mg, 135 mg, 140 mg, 145 mg, 150, 155, 160, 165, 170, 175, 180, 185, 190, 195, or 200 mg. In some embodiments the pharmaceutically acceptable salt of sumatriptan is sumatriptan succinate.

In some embodiments, a pharmaceutical composition disclosed herein comprises almotriptan or a pharmaceutically acceptable salt thereof, that is present at a dose of from about 1.0 mg to about 50 mg, including, but not limited to, about 1.0 mg to about 30 mg, about 5.0 mg to about 25 mg, about 5.0 mg to about 15 mg, about 1.0 mg to about 5.0 mg, about 5.0 mg to about 10.0 mg, about 10.0 mg to about 15 mg, about 15 mg to about 20 mg, about 20 mg to about 25 mg, about 25 mg to about 30 mg, about 35 mg to about 40 mg, about 40 mg to about 45 mg, or about 45 mg to about 50 mg. In some embodiments a pharmaceutical composition comprises almotriptan or a pharmaceutically acceptable salt thereof, that is present at a dose of from about 1.0 mg to about 50 mg, including, but not limited to, about 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, or 50 mg. In some embodiments the pharmaceutically acceptable salt of almotriptan is almotriptan malate.

In some embodiments, a pharmaceutical composition disclosed herein comprises eletriptan or a pharmaceutically acceptable salt thereof, that is present at a dose of from about 10.0 mg to about 100 mg, including, but not limited to, about 10.0 mg to about 75 mg, about 10.0 mg to about 50 mg, about 10 mg to about 30 mg, about 30 mg to about 50 mg, about 50 mg to about 70 mg, about 70 mg to about 90 mg, about 10.0 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, or about 90 mg to about 100 mg. In some embodiments a pharmaceutical composition comprises eletriptan or a pharmaceutically acceptable salt thereof, that is present at a dose of from about 10.0 mg to about 100 mg, including, but not limited to, about 10.0 mg, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, or 100 mg. In some embodiments the pharmaceutically acceptable salt of eletriptan is eletriptan hydrobromide.

In some embodiments, a pharmaceutical composition disclosed herein comprises frovatriptan or a pharmaceutically acceptable salt thereof, that is present at a dose of from about 0.5 mg to about 10.0 mg, including, but not limited to, about 0.5 mg to about 5.0 mg, about 1.0 mg to about 3.0 mg, about 0.5 mg to about 1.5 mg, about 1.5 mg to about 3.0 mg, about 3.0 mg to about 4.5 mg, about 4.5 mg to about 6.0 mg, about 6.0 mg to about 7.5 mg, about 7.5 mg to about 9.0 mg, about 9.0 mg to about 10.0 mg, about 0.5 mg to about 1.0 mg about 1.0 mg to about 2.0 mg, about 2.0 mg to about 3.0 mg, about 3.0 mg to about 4.0 mg, about 4.0 mg to about 5.0 mg, about 5.0 mg to about 6.0 mg, about 6.0 mg to about 7.0 mg, about 7.0 mg to about 8.0 mg, or about 8.0 mg to about 9.0 mg. In some embodiments a pharmaceutical composition comprises frovatriptan or a pharmaceutically acceptable salt thereof, that is present at a dose of from about 0.5 mg to about 10.0 mg, including, but not limited to, about 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, or 10.0 mg. In some embodiments the pharmaceutically acceptable salt of frovatriptan is frovatriptan succinate.

In some embodiments a pharmaceutical composition disclosed herein comprises rizatriptan or a pharmaceutically acceptable salt thereof, that is present at a dose of from about 1.0 mg to about 50 mg, including, but not limited to, about 1.0 mg to about 75 mg, about 1.0 mg to about 50 mg, about 1.0 mg to about 25 mg, about 1.0 mg to about 15 mg, about 15 mg to about 30 mg, about 30 mg to about 45 mg, about 1.0 mg to about 5.0 mg, about 5.0 mg to about 10.0 mg, about 10.0 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg. In some embodiments a pharmaceutical composition comprises rizatriptan or a pharmaceutically acceptable salt thereof, that is present at a dose of from about 1.0 mg to about 50 mg, including, but not limited to, about 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, or 50 mg. In some embodiments the pharmaceutically acceptable salt of rizatriptan is rizatriptan benzoate.

In some embodiments a pharmaceutical composition disclosed herein comprises zolmitriptan or a pharmaceutically acceptable salt thereof, that is present at a dose of from about 1.0 mg to about 25 mg, including, but not limited to, about 1.0 mg to about 15 mg, about 1.0 mg to about 10 mg, about 1.0 mg to about 7.5 mg, about 1.0 mg to about 7.0 mg, about 7.0 mg to about 14 mg, about 14 mg to about 25 mg, about 1.0 mg to about 2.5 mg, about 2.5 mg to about 5.0 mg, about 5.0 mg to about 7.5 mg, about 7.5 mg to about 10 mg, about 10 mg to about 12.5 mg, about 12.5 mg to about 15 mg, about 15 mg to about 17.5 mg, about 17.5 mg to about 20 mg, or about 20 mg to about 25 mg. In some embodiments a pharmaceutical composition comprises zolmitriptan or a pharmaceutically acceptable salt thereof, that is present at a dose of from about 1.0 mg to about 25 mg, including, but not limited to, about 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, or 25 mg.

In some embodiments a pharmaceutical composition disclosed herein comprises naratriptan or a pharmaceutically acceptable salt thereof, that is present at a dose of from about 0.5 mg to about 25 mg, including, but not limited to, about 0.5 mg to about 10 mg, about 0.5 mg to about 7.5 mg, about 0.5 mg to about 5.0 mg, about 0.5 mg to about 4.0 mg, about 0.5 mg to about 3.0 mg, about 3.0 mg to about 5.0 mg, about 5.0 mg to about 10.0 mg, about 10.0 mg to about 15 mg, about 15 mg to about 20 mg, about 20 mg to about 25 mg, about 1.0 mg to about 4.0 mg, about 4.0 mg to about 7.0 mg, or about 7.0 mg to about 10.0 mg. In some embodiments, a pharmaceutical composition comprises naratriptan or a pharmaceutically acceptable salt thereof, that is present at a dose of from about 1.0 mg to about 25 mg, including, but not limited to, about 0.5 mg, 0.6 mg, 0.7 mg, 0.8 mg, 0.9 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, or 25 mg. In some embodiments the pharmaceutically acceptable salt of naratriptan is naratriptan hydrochloride.

In some embodiments, a pharmaceutical composition comprises sumatriptan or a pharmaceutically acceptable salt thereof and promethazine or a pharmaceutically acceptable salt thereof. In some embodiments, the sumatriptan or a pharmaceutically acceptable salt thereof is present at a dose of from about 10 mg to about 200 mg, including, but not limited to, about 10.0 mg, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100, 105 mg, 110 mg, 115 mg, 120 mg, 120.5 mg, 121 mg, 121.5 mg, 122 mg, 122.5 mg, 123 mg, 123.5 mg, 124 mg, 124.5 mg, 125 mg, 125.5 mg, 126 mg, 126.5 mg, 127 mg, 127.5 mg, 128 mg, 128.5 mg, 129 mg, 129.5 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, about 25 mg to about 100 mg, about 35 mg to about 140 mg, about 70 mg to about 140 mg, about 80 mg to about 135 mg, about 10 mg to about 25 mg, about 25 mg to about 50 mg, about 50 mg to about 100 mg, about 100 mg to about 150 mg, about 150 mg to about 200 mg, about 10 mg to about 35 mg, about 35 mg to about 70 mg, about 70 mg to about 105 mg, about 105 mg to about 140 mg, about 140 mg to about 175 mg, or about 175 mg to about 200 mg. In some instances, promethazine or a pharmaceutically acceptable salt thereof is present at a dose of from about 0.5 mg to about 100 mg, including, but not limited to, about 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, about 0.5 mg to about 12.5 mg, about 12.5 mg to about 50 mg, about 50 mg to about 75 mg, about 75 mg to about 100 mg, about 0.5 mg to about 15 mg, about 15 mg to about 35 mg, about 35 mg to about 55 mg, about 55 mg to about 75 mg, or about 75 mg to about 95 mg. In some embodiments, sumatriptan or a pharmaceutically acceptable salt thereof is present in a plurality of first particulates and promethazine or a pharmaceutically acceptable salt thereof is present in a plurality of second particulates.

In some embodiments, a pharmaceutical composition disclosed herein comprises sumatriptan succinate and promethazine hydrochloride. In some embodiments, the sumatriptan succinate is present at a dose of from about 10 mg to about 200 mg, including, but not limited to, about 10.0 mg, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100, 105 mg, 110 mg, 115 mg, 120 mg, 120.5 mg, 121 mg, 121.5 mg, 122 mg, 122.5 mg, 123 mg, 123.5 mg, 124 mg, 124.5 mg, 125 mg, 125.5 mg, 126 mg, 126.5 mg, 127 mg, 127.5 mg, 128 mg, 128.5 mg, 129 mg, 129.5 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, about 25 mg to about 100 mg, about 35 mg to about 140 mg, about 70 mg to about 140 mg, about 80 mg to about 135 mg, about 10 mg to about 25 mg, about 25 mg to about 50 mg, about 50 mg to about 100 mg, about 100 mg to about 150 mg, about 150 mg to about 200 mg, about 10 mg to about 35 mg, about 35 mg to about 70 mg, about 70 mg to about 105 mg, about 105 mg to about 140 mg, about 140 mg to about 175 mg, or about 175 mg to about 200 mg. In some instances, promethazine hydrochloride is present at a dose of from about 0.5 mg to about 100 mg, including, but not limited to, from about 0.5 mg to about 100 mg, including but not limited to, about 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 31 mg, 32 mg, 33 mg, 34 mg, 35 mg, 36 mg, 37 mg, 38 mg, 39 mg, 40 mg, 41 mg, 12 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, about 0.5 mg to about 12.5 mg, about 12.5 mg to about 50 mg, about 50 mg to about 75 mg, about 75 mg to about 100 mg, about 0.5 mg to about 15 mg, about 15 mg to about 35 mg, about 35 mg to about 55 mg, about 55 mg to about 75 mg, or about 75 mg to about 95 mg. In some embodiments, sumatriptan succinate is present in a plurality of first particulates and promethazine hydrochloride is present in a plurality of second particulates.

In some aspects, a pharmaceutical composition disclosed herein comprises multiple pharmaceutically acceptable excipients contained in a plurality of first particulates and a plurality of second particulates. In some embodiments, the particulates are beads, pellets, or spherules. In some embodiments, the particulates comprise a therapeutically effective amount of a triptan or a pharmaceutically acceptable salt thereof. In some embodiments, the particulates comprise a therapeutically effective amount of an antiemetic or a pharmaceutically acceptable salt thereof. In some embodiments, the triptan and the antiemetic vary in dosages as described herein and the pharmaceutically acceptable excipients are adjusted according to the dosages of the triptan and the antiemetic.

In some embodiments, a pharmaceutical composition disclosed herein comprises a vinyl polymer that is present in a percentage by weight of the plurality of first particulates that ranges from about 0.25% to about 6.0%, including but not limited to, about 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, 5.0%, 5.25%, 5.5%, 5.75%, or 6.0%. In some embodiments, the vinyl polymer is polyvinylpyrrolidone. In some embodiments, a pharmaceutical composition disclosed herein comprises a vinyl copolymer that is present in a percentage by weight of the plurality of first particulates that ranges from about 0.25% to about 30%, including but not limited to about 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, 5.0%, 5.25%, 5.5%, 5.75%, 6.0%, 6.5%, 7.0%, 7.5%, 8.0%, 8.5%, 9.0%, 9.5%, 10.0%, 11% 12%, 13%, 14%, 15%, 16%, 17%, 18%, 19%, 20%, 21%, 22%, 23%, 24%, 25%, 26%, 27%, 28%, 29%, or 30%. In some embodiments the vinyl copolymer is a polyvinylpyrrolidone/vinyl acetate copolymer or a polyvinylpyrrolidone/polyvinyl acetate copolymer. In some embodiments, the vinyl copolymer is a vinylpyrrolidone/vinyl acetate copolymer. In some embodiments, a pharmaceutical composition disclosed herein comprises microcrystalline cellulose that is present in a percentage by weight of the plurality of first particulates that ranges from about 20% to about 90%, including, but not limited to, about 20.0%, 20.5%, 21.0%, 21.5%, 22%, 22.5%, 23%, 23.5%, 24.0%, 24.5%, 25.0%, 25.5%, 26.0%, 26.5%, 27.0%, 27.5%, 28.0%, 28.5%, 29.0%, 29.5%, 30.0%, 30.5%, 31.0%, 31.5%, 32.0%, 32.5%, 33.0%, 33.5%, 34.0%, 34.5%, 35.0%, 35.5%, 36.0%, 36.5%, 37.0%, 37.5%, 38.0%, 38.5%, 39.0%, 39.5%, 40.0%, 40.5%, 41.5%, 42.0%, 42.5%, 43.0%, 43.5%, 44.0%, 44.5%, 45.0%, 45.5%, 46.5%, 47.0%, 47.5%, 48.0%, 48.5%, 49.0%, 49.5%, 50.0%, 55%, 60%, 65%, 70%, 75%, 80%, 85%, or 90%. In some embodiments, a pharmaceutical composition disclosed herein comprises croscarmellose sodium that is present in a percentage by weight of the plurality of first particulates that ranges from about greater than 0.0% to about 5.0%, including, but not limited to, about greater than 0.0%, 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, or 5.0%. In some embodiments, a pharmaceutical composition disclosed herein comprises magnesium stearate that is present in a percentage by weight of the plurality of first particulates that ranges from about 0.2% to about 5.0%, including, but not limited to, about 0.2%, 0.25%, 0.3%, 0.35%, 0.4%, 0.45%, 0.5%, 0.55%, 0.6%, 0.7%, 0.75%, 0.8%, 0.85%, 0.9%, 0.95%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, or 5.0%. In some embodiments, a pharmaceutical composition disclosed herein comprises talc that is present in a percentage by weight of the plurality of first particulates that ranges from about 0.1% to about 5.0%, including, but not limited to, about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.25%, 2.5%, 2.75%, 3.0%, 3.25%, 3.5%, 3.75%, 4.0%, 4.25%, 4.5%, 4.75%, or 5.0%.

In some embodiments, a pharmaceutical composition disclosed herein comprises a plurality of first particulates comprising polyvinylpyrrolidone, microcrystalline cellulose, croscarmellose sodium, magnesium stearate, and talc; and a plurality of second particulates comprising microcrystalline cellulose and croscarmellose sodium. In some embodiments, polyvinylpyrrolidone disclosed herein is present in a percentage by weight of the plurality of first particulates that ranges from about 0.25% to about 6.0%, including but not limited to, about 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, 5.0%, 5.25%, 5.5%, 5.75%, or 6.0%.

In some embodiments, microcrystalline cellulose is present in a percentage by weight of the plurality of first particulates that ranges from about 20% to about 90%, including, but not limited to, about 20.0%, 20.5%, 21.0%, 21.5%, 22%, 22.5%, 23%, 23.5%, 24.0%, 24.5%, 25.0%, 25.5%, 26.0%, 26.5%, 27.0%, 27.5%, 28.0%, 28.5%, 29.0%, 29.5%, 30.0%, 30.5%, 31.0%, 31.5%, 32.0%, 32.5%, 33.0%, 33.5%, 34.0%, 34.5%, 35.0%, 35.5%, 36.0%, 36.5%, 37.0%, 37.5%, 38.0%, 38.5%, 39.0%, 39.5%, 40.0%, 40.5%, 41.5%, 42.0%, 42.5%, 43.0%, 43.5%, 44.0%, 44.5%, 45.0%, 45.5%, 46.5%, 47.0%, 47.5%, 48.0%, 48.5%, 49.0%, 49.5%, 50.0%, 55%, 60%, 65%, 70%, 75%, 80%, 85%, or 90%. In some embodiments, croscarmellose sodium is present in a percentage by weight of the plurality of first particulates that ranges from about greater than 0.0% to about 5.0%, including, but not limited to, about greater than 0.0%, 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, or 5.0%. In some embodiments, magnesium stearate is present in a percentage by weight of the plurality of first particulates that ranges from about 0.2% to about 5.0%, including, but not limited to, about 0.2%, 0.25%, 0.3%, 0.35%, 0.4%, 0.45%, 0.5%, 0.55%, 0.6%, 0.7%, 0.75%, 0.8%, 0.85%, 0.9%, 0.95%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, or 5.0%. In some embodiments, talc is present in a percentage by weight of the plurality of first particulates that ranges from about 0.1% to about 5.0%, including, but not limited to, about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.25%, 2.5%, 2.75%, 3.0%, 3.25%, 3.5%, 3.75%, 4.0%, 4.25%, 4.5%, 4.75%, or 5.0%.

In some embodiments, microcrystalline cellulose disclosed herein is present in a percentage by weight of the plurality of second particulates that ranges from about 20% to about 90%, including, but not limited to, about 20.0%, 20.5%, 21.0%, 21.5%, 22%, 22.5%, 23%, 23.5%, 24.0%, 24.5%, 25.0%, 25.5%, 26.0%, 26.5%, 27.0%, 27.5%, 28.0%, 28.5%, 29.0%, 29.5%, 30.0%, 30.5%, 31.0%, 31.5%, 32.0%, 32.5%, 33.0%, 33.5%, 34.0%, 34.5%, 35.0%, 35.5%, 36.0%, 36.5%, 37.0%, 37.5%, 38.0%, 38.5%, 39.0%, 39.5%, 40.0%, 40.5%, 41.5%, 42.0%, 42.5%, 43.0%, 43.5%, 44.0%, 44.5%, 45.0%, 45.5%, 46.5%, 47.0%, 47.5%, 48.0%, 48.5%, 49.0%, 49.5%, 50.0%, 51%, 52%, 53%, 54%, 55%, 56%, 57%, 58%, 59%, 60%, 65%, 70%, 75%, 80%, 85%, or 90%. In some embodiments, croscarmellose sodium is present in a percentage by weight of the plurality of first particulates that ranges from about greater than 0.0% to about 5.0%, including, but not limited to, about greater than 0.0%, 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, or 5.0%.

In some embodiments, a pharmaceutical composition disclosed herein comprises a plurality of first particulates containing microcrystalline cellulose and polyvinylpyrrolidone, wherein the relative ratio by percentage weight of each of microcrystalline cellulose: polyvinylpyrrolidone is about (3 to 120):1, such as about 3:1, 4:1, 5:1, 6:1, 7:1, 8:1, 9:1, 10:1, 11:1, 12:1, 13:1, 14:1 15:1, 16:1, 17:1, 18:1, 19:1, 20:1, 25:1, 30:1, 35:1, 40:1, 45:1, 50:1, 55:1, 60:1, 70:1, 80:1, 90:1, 100:1, 110:1, or 120:1.

In some embodiments, a pharmaceutical composition disclosed herein comprises a plurality of first particulates containing a triptan or a pharmaceutically acceptable salt thereof and polyvinylpyrrolidone, wherein the relative ratio by percentage weight of each of the triptan or a pharmaceutically acceptable salt thereof: polyvinylpyrrolidone about (8 to 150):1, such as about 8:1, 9:1, 10:1, 11:1 12:1, 13:1 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 42:1, 44:1, 46:1, 48:1, 50:1, 55:1, 60:1, 65:1, 70:1, 75:1, 80:1, 90:1, 95:1, 100:1, 110:1, 120:1, 130:1, 140:1, or 150:1.

In some aspects, a pharmaceutical composition disclosed herein comprises a plurality of first particulates comprising a therapeutically effective amount of a first pharmaceutically active agent and one or more first pharmaceutically acceptable excipients, and a plurality of second particulates comprising a therapeutically effective amount of a second pharmaceutically active agent and one or more second pharmaceutically acceptable excipients. In some embodiments, a pharmaceutical composition disclosed herein comprises a plurality of first particulates comprising a therapeutically effective amount of a triptan and one or more first pharmaceutically acceptable excipients, and a plurality of second particulates comprising a therapeutically effective amount of an antiemetic and one or more second pharmaceutically acceptable excipients, wherein the one or more first pharmaceutically acceptable excipients comprises a vinyl polymer or vinyl copolymer. In some embodiments, a pharmaceutical composition disclosed herein comprises a plurality of first particulates comprising a therapeutically effective amount of sumatriptan or a pharmaceutically acceptable salt thereof and one or more first pharmaceutically acceptable excipients; and a plurality of second particulates comprising a therapeutically effective amount of promethazine or a pharmaceutically acceptable salt thereof and one or more second pharmaceutically acceptable excipients, wherein the one or more first pharmaceutically acceptable excipients comprises a vinyl polymer or vinyl copolymer.

In some embodiments, a pharmaceutical composition disclosed herein comprises a plurality of first particulates comprising a therapeutically effective amount of sumatriptan or a pharmaceutically acceptable salt thereof and one or more first pharmaceutically acceptable excipients; and a plurality of second particulates comprising a therapeutically effective amount of promethazine or a pharmaceutically acceptable salt thereof and one or more second pharmaceutically acceptable excipients; wherein the one or more first pharmaceutically acceptable excipients comprises polyvinylpyrrolidone. In some embodiments, a pharmaceutical composition disclosed herein comprises a plurality of first particulates comprising a therapeutically effective amount of sumatriptan or a pharmaceutically acceptable salt thereof, polyvinylpyrrolidone, microcrystalline cellulose, croscarmellose sodium, magnesium stearate, and talc; and a plurality of second particulates comprising a therapeutically effective amount of promethazine or a pharmaceutically acceptable salt thereof, microcrystalline cellulose, and croscarmellose sodium. In some embodiments, a pharmaceutical composition disclosed herein comprises: a plurality of first particulates comprising about 10-300 mg, for example about: 50-150 mg, 10-200 mg, 25-200 mg, 50-200 mg, 60-120, 70-110, 80-100, or 85-95 mg of sumatriptan or a pharmaceutically acceptable salt thereof, about 0.1-20 mg, for example about: 1-10 mg, 0.1-10 mg, 0.1-9 mg, 0.1-8 mg, 0.1-7 mg, 0.1-6 mg, 0.1-5 mg, 1-7 mg, 2-6 mg, 3-5 mg, or 3.5-4.5 mg of polyvinylpyrrolidone, about 10-300 mg, for example about: 50-150 mg, 10-200 mg, 25-200 mg, 50-200 mg, 50-100 mg, 60-80 mg, 65-75 mg, or 70-80 mg of microcrystalline cellulose, about 0.1-20 mg, for example about: 1-10 mg, 0.1-10 mg, 0.1-9 mg, 0.1-8 mg, 0.1-7 mg, 0.1-6 mg, 0.1-5 mg, 1-7 mg, 2-6 mg, 3-5 mg, or 3.5-4.5 mg of croscarmellose sodium, about 0.1-10 mg, for example about: 0.1-5 mg, 0.1-9 mg, 0.1-8 mg, 0.1-7 mg, 0.1-6 mg, 0.1-4 mg, 0.1-3 mg, 0.1-2 mg, 0.5-1.5 mg, or 0.8-1.2 mg of magnesium stearate, and about 0.1-10 mg, for example about: 0.1-5 mg, 0.1-9 mg, 0.1-8 mg, 0.1-7 mg, 0.1-6 mg, 0.1-4 mg, 0.1-3 mg, 0.5-3 mg, 1-3 mg, 1.5-2.5 mg, or 1.8-2.4 mg of talc; and a plurality of second particulates comprising about 1-100 mg, for example about: 10-50 mg, 10-60 mg, 10-70 mg, 10-80 mg, 10-90 mg, 15-50 mg, 15-45 mg, 15-40 mg, 15-35 mg, 10-40 mg, 10-30 mg, 20-40 mg, 20-30 mg, 22-28 mg, or 24-26 mg of promethazine or a pharmaceutically acceptable salt thereof, about 1-100 mg, for example about: 10-50 mg, 10-60 mg, 10-70 mg, 10-80 mg, 10-90 mg, 15-50 mg, 15-45 mg, 15-40 mg, 15-35 mg, 10-40 mg, 10-30 mg, 20-40 mg, 20-30 mg, 22-26 mg, or 23-25 mg of microcrystalline cellulose, and about 0.1-10 mg, for example about: 0.1-5 mg, 0.1-9 mg, 0.1-8 mg, 0.1-7 mg, 0.1-6 mg, 0.1-4 mg, 0.1-3 mg, 0.1-2 mg, 0.5-1.5 mg, or 0.8-1.2 mg of croscarmellose sodium. In some embodiments, a pharmaceutical composition disclosed herein comprises: a plurality of first particulates comprising about: 90, 10, 15, 20, 25, 30, 35, 40, 45, 50, 55, 60, 65, 70, 75, 80, 85, 95, 100, 105, 110, 115, 120, 125, 130, 135, 140, 145, 150, 160, 170, 180, 190, 200, 210, 220, 230, 240, 250, 260, 270, 280, 290, or 300 mg of sumatriptan or a pharmaceutically acceptable salt thereof, about: 4, 4.2, 0.1, 0.5, 1, 1.5, 2, 2.5, 3, 3.5, 4.5, 5, 5.5, 6, 6.5, 7, 7.5, 8, 8.5, 9, 9.5, 10, 12, 14, 16, 18, or 20 mg of polyvinylpyrrolidone, about: 72, 72.45, 10, 15, 20, 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, 250, 260, 270, 280, 290, or 300 mg of microcrystalline cellulose, about: 4, 4.2, 0.1, 0.5, 1, 1.5, 2, 2.5, 3, 3.5, 4.5, 5, 5.5, 6, 6.5, 7, 7.5, 8, 8.5, 9, 9.5, 10, 12, 14, 16, 18, or 20 mg of croscarmellose sodium, about: 1, 1.05, 0.1, 0.2, 0.4, 0.6, 0.8, 1.2, 1.4, 1.6, 1.8, 2, 2.5, 3, 3.5, 4, 4.5, 5, 5.5, 6, 6.5, 7, 7.5, 8, 8.5, 9, 9.5, or 10 mg of magnesium stearate, and about: 2, 2.1, 0.1, 0.2, 0.4, 0.6, 0.8, 1.2, 1.4, 1.6, 1.8, 2, 2.5, 3, 3.5, 4.5, 5, 5.5, 6, 6.5, 7, 7.5, 8, 8.5, 9, 9.5, or 10 mg of talc; and a plurality of second particulates comprising about: 25, 1, 2, 4, 6, 8, 10, 12, 14, 16, 18, 20, 21, 22, 23, 24, 26, 27, 28, 39, 30, 35, 40, 45, 50, 55, 60, 65, 70, 75, 80, 85, 95, or 100 mg of promethazine or a pharmaceutically acceptable salt thereof, about: 24, 2, 4, 6, 8, 10, 12, 14, 16, 18, 20, 21, 22, 23, 25, 26, 27, 28, 39, 30, 35, 40, 45, 50, 55, 60, 65, 70, 75, 80, 85, 95, or 100 mg of microcrystalline cellulose, and about: 1, 0.1, 0.2, 0.4, 0.6, 0.8, 1.2, 1.4, 1.6, 1.8, 2, 2.5, 3, 3.5, 4, 4.5, 5, 5.5, 6, 6.5, 7, 7.5, 8, 8.5, 9, 9.5, or 10 mg of croscarmellose sodium.

In some aspects, a pharmaceutical composition disclosed herein comprises a plurality of first particulates comprising a therapeutically effective amount of sumatriptan succinate, polyvinylpyrrolidone, microcrystalline cellulose, croscarmellose sodium, magnesium stearate, and talc; and a plurality of second particulates comprising a therapeutically effective amount of promethazine hydrochloride, microcrystalline cellulose, and croscarmellose sodium. In some embodiments, a pharmaceutical composition disclosed herein comprises: a plurality of first particulates comprising about 10-300 mg, for example about: 50-150 mg, 10-200 mg, 25-200 mg, 50-200 mg, 60-120, 70-110, 80-100, or 85-95 mg of sumatriptan succinate, about 0.1-20 mg, for example about: 1-10 mg, 0.1-10 mg, 0.1-9 mg, 0.1-8 mg, 0.1-7 mg, 0.1-6 mg, 0.1-5 mg, 1-7 mg, 2-6 mg, 3-5 mg, or 3.5-4.5 mg of polyvinylpyrrolidone, about 10-300 mg, for example about: 50-150 mg, 10-200 mg, 25-200 mg, 50-200 mg, 50-100 mg, 60-80 mg, 65-75 mg, or 70-80 mg of microcrystalline cellulose, about 0.1-20 mg, for example about: 1-10 mg, 0.1-10 mg, 0.1-9 mg, 0.1-8 mg, 0.1-7 mg, 0.1-6 mg, 0.1-5 mg, 1-7 mg, 2-6 mg, 3-5 mg, or 3.5-4.5 mg of croscarmellose sodium, about 0.1-10 mg, for example about: 0.1-5 mg, 0.1-9 mg, 0.1-8 mg, 0.1-7 mg, 0.1-6 mg, 0.1-4 mg, 0.1-3 mg, 0.1-2 mg, 0.5-1.5 mg, or 0.8-1.2 mg of magnesium stearate, and about 0.1-10 mg, for example about: 0.1-5 mg, 0.1-9 mg, 0.1-8 mg, 0.1-7 mg, 0.1-6 mg, 0.1-4 mg, 0.1-3 mg, 0.5-3 mg, 1-3 mg, 1.5-2.5 mg, or 1.8-2.4 mg of talc; and a plurality of second particulates comprising about 1-100 mg, for example about: 10-50 mg, 10-60 mg, 10-70 mg, 10-80 mg, 10-90 mg, 15-50 mg, 15-45 mg, 15-40 mg, 15-35 mg, 10-40 mg, 10-30 mg, 20-40 mg, 20-30 mg, 22-28 mg, or 24-26 mg of promethazine hydrochloride, about 1-100 mg, for example about: 10-50 mg, 10-60 mg, 10-70 mg, 10-80 mg, 10-90 mg, 15-50 mg, 15-45 mg, 15-40 mg, 15-35 mg, 10-40 mg, 10-30 mg, 20-40 mg, 20-30 mg, 22-26 mg, or 23-25 mg of microcrystalline cellulose, and about 0.1-10 mg, for example about: 0.1-5 mg, 0.1-9 mg, 0.1-8 mg, 0.1-7 mg, 0.1-6 mg, 0.1-4 mg, 0.1-3 mg, 0.1-2 mg, 0.5-1.5 mg, or 0.8-1.2 mg of croscarmellose sodium. In some embodiments, a pharmaceutical composition disclosed herein comprises: a plurality of first particulates comprising about: 90, 10, 15, 20, 25, 30, 35, 40, 45, 50, 55, 60, 65, 70, 75, 80, 85, 95, 100, 105, 110, 115, 120, 125, 130, 135, 140, 145, 150, 160, 170, 180, 190, 200, 210, 220, 230, 240, 250, 260, 270, 280, 290, or 300 mg of sumatriptan succinate, about: 4, 4.2, 0.1, 0.5, 1, 1.5, 2, 2.5, 3, 3.5, 4.5, 5, 5.5, 6, 6.5, 7, 7.5, 8, 8.5, 9, 9.5, 10, 12, 14, 16, 18, or 20 mg of polyvinylpyrrolidone, about: 72, 72.45, 10, 15, 20, 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, 250, 260, 270, 280, 290, or 300 mg of microcrystalline cellulose, about: 4, 4.2, 0.1, 0.5, 1, 1.5, 2, 2.5, 3, 3.5, 4.5, 5, 5.5, 6, 6.5, 7, 7.5, 8, 8.5, 9, 9.5, 10, 12, 14, 16, 18, or 20 mg of croscarmellose sodium, about: 1, 1.05, 0.1, 0.2, 0.4, 0.6, 0.8, 1.2, 1.4, 1.6, 1.8, 2, 2.5, 3, 3.5, 4, 4.5, 5, 5.5, 6, 6.5, 7, 7.5, 8, 8.5, 9, 9.5, or 10 mg of magnesium stearate, and about: 2, 2.1, 0.1, 0.2, 0.4, 0.6, 0.8, 1.2, 1.4, 1.6, 1.8, 2, 2.5, 3, 3.5, 4.5, 5, 5.5, 6, 6.5, 7, 7.5, 8, 8.5, 9, 9.5, or 10 mg of talc; and a plurality of second particulates comprising about: 25, 1, 2, 4, 6, 8, 10, 12, 14, 16, 18, 20, 21, 22, 23, 24, 26, 27, 28, 39, 30, 35, 40, 45, 50, 55, 60, 65, 70, 75, 80, 85, 95, or 100 mg of promethazine hydrochloride, about: 24, 2, 4, 6, 8, 10, 12, 14, 16, 18, 20, 21, 22, 23, 25, 26, 27, 28, 39, 30, 35, 40, 45, 50, 55, 60, 65, 70, 75, 80, 85, 95, or 100 mg of microcrystalline cellulose, and about: 1, 0.1, 0.2, 0.4, 0.6, 0.8, 1.2, 1.4, 1.6, 1.8, 2, 2.5, 3, 3.5, 4, 4.5, 5, 5.5, 6, 6.5, 7, 7.5, 8, 8.5, 9, 9.5, or 10 mg of croscarmellose sodium.

In some aspects, a pharmaceutical composition disclosed herein comprises a plurality of first particulates comprising from about 40% to about 80% by weight of sumatriptan succinate, from about 0.5% to about 5% by weight of polyvinylpyrrolidone, from about 20% to about 60% by weight of microcrystalline cellulose, from about 0.5% to about 5% by weight of croscarmellose sodium, from about 0.1% to about 5% by weight of magnesium stearate, and from about 0.1% to about 5% by weight of talc; and a plurality of second particulates comprising from about 30% to about 70% by weight of promethazine hydrochloride, from about 20% to about 70% by weight of microcrystalline cellulose, and from about 0.5% to about 5% by weight of croscarmellose sodium. In some embodiments, a pharmaceutical composition disclosed herein comprises a plurality of first particulates comprising about: 60%, 80%, 75%, 70%, 65%, 55%, 50%, 45%, or 40% by weight of sumatriptan succinate, about: 2%, 0.5%, 0.6%, 0.7%, 0.8%, 0.9%, 1%, 1.2%, 1.4%, 1.6%, 1.8%, 2.2%, 2.5%, 3%, 3.5%, 4%, 4.5%, or 5% by weight of polyvinylpyrrolidone, about: 34.5%, 20%, 25%, 30%, 35%, 40%, 45%, 50%, 55%, or 60% by weight of microcrystalline cellulose, about: 2%, 0.5%, 0.6%, 0.7%, 0.8%, 0.9%, 1%, 1.2%, 1.4%, 1.6%, 1.8%, 2.2%, 2.5%, 3%, 3.5%, 4%, 4.5%, or 5% by weight of croscarmellose sodium, about: 0.5%, 0.1%, 0.2%, 0.3%, 0.4%, 0.6%, 0.7%, 0.8%, 0.9%, 1%, 1.2%, 1.4%, 1.6%, 1.8%, 2%, 2.2%, 2.5%, 3%, 3.5%, 4%, 4.5%, or 5% by weight of magnesium stearate, and about: 1%, 0.1%, 0.2%, 0.3%, 0.4%, 0.6%, 0.7%, 0.8%, 0.9%, 1.2%, 1.4%, 1.6%, 1.8%, 2%, 2.2%, 2.5%, 3%, 3.5%, 4%, 4.5%, or 5% by weight of talc; and a plurality of second particulates comprising about: 50%, 30%, 35%, 40%, 45%, 55%, 60%, 65%, or 70% by weight of promethazine hydrochloride, about: 48%, 30%, 35%, 40%, 45%, 50%, 55%, 60%, 65%, or 70% by weight of microcrystalline cellulose, and about: 2%, 0.5%, 0.6%, 0.7%, 0.8%, 0.9%, 1%, 1.2%, 1.4%, 1.6%, 1.8%, 2.2%, 2.5%, 3%, 3.5%, 4%, 4.5%, or 5% by weight of croscarmellose sodium. In some embodiments, a pharmaceutical composition disclosed herein comprises a plurality of first particulates comprising from about 84 mg to about 126 mg of sumatriptan succinate, from about 1.05 mg to about 10.5 mg of polyvinylpyrrolidone, from about 42 mg to about 126 mg of microcrystalline cellulose, from about 1.05 mg to about 10.5 mg of croscarmellose sodium, from about 0.525 mg to about 10.5 mg of magnesium stearate, and from about 2.1 mg to about 10.5 mg of talc; and a plurality of second particulates comprising from about 20 mg to about 30 mg of promethazine hydrochloride, from about 10 mg to about 30 mg of microcrystalline cellulose, and from about 0.25 mg to about 2.5 mg of croscarmellose sodium. In some embodiments, a pharmaceutical composition disclosed herein comprises a plurality of first particulates comprising about 126 mg of sumatriptan succinate, about 4.2 mg of polyvinylpyrrolidone, about 72.45 mg of microcrystalline cellulose, about 4.2 mg of croscarmellose sodium, about 1.05 mg of magnesium stearate, and about 2.1 mg of talc; and a plurality of second particulates comprising about 25 mg of promethazine hydrochloride, about 24 mg of microcrystalline cellulose, and about 1 mg of croscarmellose sodium.

In some embodiments, a pharmaceutical composition disclosed herein is a fast release pharmaceutical composition. In some embodiments, a pharmaceutical composition disclosed herein is wherein at least about 80% of both the sumatriptan or a pharmaceutically acceptable salt thereof and the promethazine or a pharmaceutically acceptable salt thereof are released within about 15 minutes as measured by contact of the pharmaceutical composition with a dissolution fluid in a USP Apparatus 1 (Basket) rotating at 100 rpm. In some embodiments, a pharmaceutical composition disclosed herein comprises: a plurality of first particulates, wherein each of the first particulates comprises sumatriptan or a pharmaceutically acceptable salt thereof; and a plurality of second particulates, wherein each of the second particulates comprises promethazine or a pharmaceutically acceptable salt thereof, wherein at least about 80% of both the sumatriptan or a pharmaceutically acceptable salt thereof and the promethazine or a pharmaceutically acceptable salt thereof are released within about 15 minutes as measured by contact of the pharmaceutical composition with a dissolution fluid in a USP Apparatus 1 (Basket) rotating at 100 rpm.

In some embodiments, a pharmaceutical composition disclosed herein is stable for at least about: 30 days, 60 days, 90 days, 6 months, 1 year, 18 months, 2 years, 3 years, 4 years, or 5 years, for example about 80%-100% such as about: 80%, 90%, 95%, or 100% of each active pharmaceutical agent in the pharmaceutical composition is stable, e.g., as measured by High Performance Liquid Chromatography (HPLC) such as the HPLC method in Example 5. In some embodiments, about 80%-100% (e.g., about: 90%-100% or 95-100%) of a 5HT1B receptor agonist (e.g., triptan such as sumatriptan) or a pharmaceutically acceptable salt thereof (e.g., sumatriptan succinate) in a pharmaceutical composition disclosed herein is stable for at least about: 30, 60, 90, 180, 360, 540, or 720 days, for example greater than 90 days, which can be measured by HPLC such as the method in Example 5. In some embodiments, about: 80%, 85%, 90%, 95%, or 100% (e.g., about 95%) of the 5HT1B receptor agonist (e.g., triptan such as sumatriptan) or the pharmaceutically acceptable salt thereof (e.g., sumatriptan succinate) is stable for 30 days or more, which can be measured by HPLC such as the method in Example 5. In some embodiments, about 80%-100% (e.g., about: 90%-100% or 95-100%) of an antiemetic (e.g. promethazine or a pharmaceutically acceptable salt thereof such as promethazine hydrochloride) in a pharmaceutical composition disclosed herein is stable for at least about: 30, 60, 90, 180, 360, 540, or 720 days, for example greater than 90 days, which can be measured by HPLC such as the method in Example 5. In some embodiments, about: 80%, 85%, 90%, 95%, or 100% (e.g., about 100%) of the antiemetic (e.g. promethazine or a pharmaceutically acceptable salt thereof such as promethazine hydrochloride) is stable for 30 days or more, which can be measured by HPLC such as the method in Example 5.

Dosage Forms

In some aspects, a pharmaceutical composition as disclosed herein comprises one or more pluralities of particulates. Amounts and weight ratios disclosed herein for particulates and their components provide an advantageous feature for the treatment of a headache (e.g., a migraine or cluster headache). Amounts and weight ratios disclosed herein for particulates and their components also provide an advantageous feature for the treatment of nausea associated with a migraine and/or vomiting associated with a migraine. In some embodiments, the one or more pluralities of particulates are enclosed in a discrete unit. In some embodiments, the discrete unit is a capsule. In some embodiments, the capsule is formed using materials which include, but are not limited to, natural or synthetic gelatin, pectin, casein, collagen, protein, modified starch, polyvinylpyrrolidone, acrylic polymers, cellulose derivatives, or combinations thereof. In some embodiments, the capsule is formed using preservatives, coloring and opacifying agents, flavorings and sweeteners, sugars, gastroresistant substances, or combinations thereof. In some embodiments, the discrete unit is a packet. In some embodiments, the capsule is coated. In some embodiments, the coating covering the capsule includes, but is not limited to, immediate release coatings, protective coatings, enteric or delayed release coatings, sustained release coatings, barrier coatings, seal coatings, or combinations thereof. In some embodiments, a capsule herein is hard or soft. In some embodiments, the capsule is seamless. In some embodiments, the capsule is broken such that the particulates are sprinkled on soft foods and swallowed without chewing. In some embodiments, the shape and size of the capsule also vary. Examples of capsule shapes include, but are not limited to, round, oval, tubular, oblong, twist off, or a non-standard shape. The size of the capsule may vary according to the volume of the particulates. In some embodiments, the size of the capsule is adjusted based on the volume of the particulates. Hard or soft gelatin capsules may be manufactured in accordance with conventional methods as a single body unit comprising the standard capsule shape. A single-body soft gelatin capsule typically may be provided, for example, in sizes from 3 to 22 minims (1 minims being equal to 0.0616 ml) and in shapes of oval, oblong or others. The gelatin capsule may also be manufactured in accordance with conventional methods, for example, as a two-piece hard gelatin capsule, sealed or unsealed, typically in standard shape and various standard sizes, conventionally designated as (000), (00), (0), (1), (2), (3), (4), and (5). The largest number corresponds to the smallest size. In some embodiments, a pharmaceutical composition disclosed herein (e.g., capsule) is swallowed as a whole. In some embodiments, a pharmaceutical composition disclosed herein (e.g., capsule) does not completely disintegrate in mouth within about: 2, 3, 4, 5, 6, 7, 8, 9, 10, 11, 12, 13, 14, 15, 16, 17, 18, 19 or 20 minutes. In some embodiments, a pharmaceutical composition disclosed herein is not a film. In some embodiments, a pharmaceutical composition disclosed herein is not for buccal administration. In some embodiments, a pharmaceutical composition disclosed herein (e.g., capsule) dissolves in stomach or intestine.

In some embodiments, a capsule includes a plurality of first particulates having a total weight of about 200 mg to about 220 mg and a plurality of second particulates having a total weight of about 45 mg to about 55 mg. The plurality of first particulates includes a first active pharmaceutical ingredient and one or more first pharmaceutically acceptable expedients. Exemplary first active pharmaceutical ingredients include triptans, e.g., sumatriptan. Exemplary first active pharmaceutical ingredients include antiemetics, e.g., promethazine. In some cases, the particulates are sorted through #16 and #30 nested mesh screens, resulting in particulates between 595 microns and 1190 microns in diameter. In some cases, the particulates of from about 595 microns to about 707 microns, from about 707 microns to about 841 microns, from about 841 microns to about 1000 microns, or from about 1000 microns to about 1190 microns in diameter. In some cases, the plurality of first particulates is about 208 or about 212 mg. In some cases, the plurality of first particulates comprises about 50 mg or 51 mg of promethazine.

In some embodiments, a capsule for holding a plurality of first particulates and a plurality of second particulates has a net weight of ranging from 28 mg to 107 mg, e.g., from about 90 mg to about 102 mg, about 100-114 mg, about 103-117 mg, about 76-86 mg, about 71-81 mg, about 61-71 mg, about 57-65 mg, about 45-51 mg, about 37-43 mg, about 35-41 mg, or about 26-30 mg. In some cases, the capsule has a net weight of about: 96 mg, 107 mg, 110 mg, 81 mg, 76 mg, 66 mg, 61 mg, 48 mg, 40 mg, 38 mg, or 28 mg. In some cases, a capsule for holding a plurality of first particulates and a plurality of second particulates has a volume ranging from about 0.1 to 0.8 ml, e.g., about 0.6 ml to about 0.8 ml, about 0.4-0.6 ml, about 0.3-0.5 ml, about 0.2-0.4 ml, about 0.1-0.3 ml, or about 0.05-0.25 ml. In some cases, the capsule has a volume of about: 0.7 ml, 0.8 ml, 0.5 ml, 0.4 ml, 0.35 ml, 0.3 ml, 0.25 ml, 0.2 ml, 0.15 ml, or 0.1 ml. In some cases, a body of the capsule ranges from about 9-20 mm long, e.g., about 17 mm to about 20 mm long, about 17-19 mm long, about 16-20 mm long, about 15-19 mm long, about 14-18 mm long, about 13-17 mm long, about 12-16 mm long, about 11-15 mm long, about 10-14 mm long, about 9-13 mm long, about 9-12 mm long, about 9-11 mm long, or about 9-10 mm long. In some cases, the body of the capsule is about: 18 mm long, 17 mm long, 16 mm long, 15 mm long, 14 mm long, 13 mm long, 12 mm long, 11 mm long, 10 mm long, or 9 mm long. In some cases, a cap of the capsule ranges from about 6-12 mm long, e.g., about 10 mm to 12 mm long, about 9-11 mm long, about 8-10 mm long, about 7-9 mm long, or about 6-8 mm long. In some cases, the cap of the capsule is about: 11 mm long, 10 mm long, 9 mm long, 8 mm long, 7 mm long, or 6 mm long. In some cases, the body of the capsule has an external diameter ranging from about 4-9 mm, e.g., about 6 mm to about 8 mm, about 7-9 mm, about 7-8 mm, about 5-7 mm, or about 4-6 mm. In some cases, the body of the capsule has an external diameter of about: 9 mm, 8 mm, 7 mm, 6 mm, 5 mm, or 4 mm. In some cases, a cap of the capsule has an external diameter ranging from about 4-9 mm, e.g., about 7 mm to about 9 mm, about 6-9 mm, about 7-8 mm, about 5-7 mm, or about 4-6 mm. In some cases, the cap of the capsule has an external diameter of about 8 mm, 9 mm, 7 mm, 6 mm, 5 mm, or 4 mm. In some cases, an overall closed length of the capsule ranges from about 10 to 24 mm, e.g., about 20 mm to 24 mm, or about: 21 to 23 mm, 20 to 22 mm, 19 to 21 mm, 18 to 20 mm, 17 to 19 mm, 16 to 18 mm, 15 to 17 mm, 14 to 16 mm, 13 to 15 mm, 12 to 14 mm, 11 to 13 mm, or 10 to 12 mm. In some cases, the overall closed length of the capsule is about: 22 mm, 24 mm, 23 mm, 21 mm, 20 mm, 19 mm, 18 mm, 17 mm, 16 mm, 15 mm, 14 mm, 13 mm, 12 mm, 11 mm, or 10 mm. In some cases, the capsule has a capacity of about 50-800 mg, e.g., about: 400-800 mg, 350-450 mg, 300-500 mg, 300-400 mg, 250-350 mg, 200-300 mg, 200-250 mg, 150-200 mg, 100-200 mg, 100-150 mg, 50-100 mg, 450 mg, 425 mg, 400 mg, 375 mg, 350 mg, 325 mg, 300 mg, 275 mg, 250 mg, 225 mg, 200 mg, 175 mg, 150 mg, 125 mg, 100 mg, or 75 mg, and a powder density of about 0.6 to about 1.2 g/ml, e.g., about: 0.6 g/ml, 0.8 g/ml, 1 g/ml, or 1.2 g/ml. In some cases, each of the first particulates and/or the second particulates in the capsule is in the shape of a bead or pellet or spherule. In some cases, the first particulates and/or the second particulates are in off-white color. In some cases, the capsule is oblong. In some cases, the capsule is in orange color. In some cases, the capsule is in white color. In some aspects, a pharmaceutical composition as disclosed herein is in the form of a tablet, film, or particulates.

Particulates

In some aspects, pharmaceutical compositions disclosed herein contain particulates that vary in form. In some embodiments, particulates are beads, granules, powders, pastes, spherules, or pellets (e.g., micropellets, or minipellets). In some embodiments, the particulates are in different sizes. In some embodiments, the diameter of the particulates range from greater than 0.1 mm to about 2.0 mm, including, but not limited to, about 0.05 mm, 0.06 mm, 0.07 mm, 0.08 mm, 0.09 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.85 mm, 0.9 mm, 0.95 mm, 1.0 mm, 1.05 mm, 1.1 mm, 1.15 mm, 1.2 mm, 1.25 mm, 1.3 mm, 1.35 mm, 1.4 mm, 1.45 mm, 1.5 mm, 1.55 mm, 1.6 mm, 1.7 mm, 1.8 mm, 1.9 mm, or 2.0 mm. In some embodiments, the diameter of the particulates range from 0.1 mm to about 2.0 mm, including, but not limited to about 0.5 mm to about 1.5 mm, about 0.595 mm to about 1.19 mm. In some embodiments, the particulate size ranges from 0.60 to 0.85 mm. In some embodiments, the particulates are beads, spherules, or pellets. In some embodiments, the particulate size is up to 2.5 mm, to a maximum size of 2.8 mm for drug products labeled for sprinkle.

In some aspects, a pharmaceutical composition disclosed herein comprises a plurality of first particulates and a plurality of second particulates. In some embodiments, the first and second particulates have about the same diameter. In some embodiments, the first particulates and second particulates are beads, spherules, or pellets. In some embodiments, a pharmaceutical composition comprises a plurality of first particulates and a plurality of second particulates, wherein the diameters of the first particulates and the second particulates range from about 0.1 mm to about 2.0 mm, including, but not limited to, about 0.5 mm to about 1.5 mm, about 0.595 mm to about 1.19 mm, about 0.1 mm to about 0.25 mm, about 0.25 mm to about 0.5 mm, about 0.5 mm to about 0.75 mm, about 0.75 mm to about 1.0 mm, about 1.0 mm to about 1.25 mm, about 1.25 mm to about 1.5 mm, about 1.5 mm to about 1.75 mm, or about 1.75 mm to about 2.0 mm. In some embodiments, the diameters of the first particulates and the second particulates are the same. In some embodiments, the diameters of the first particulates and the second particulates are different. In some embodiments, a pharmaceutical composition comprises from about 150 mg to about 400 mg of a plurality of first particulates, including, but not limited to, about 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, or 400 mg. In some embodiments, a pharmaceutical composition comprises from about 150 mg to about 400 mg of a plurality of first particulates, including, but not limited to, about 175 mg to about 300 mg, about 200 mg to about 250 mg, about 200 mg to about 220 mg, about 150 mg to about 175 mg, about 175 mg to about 200 mg, about 200 mg to about 225 mg, about 225 mg to about 250 mg, about 250 mg to about 275 mg, about 275 mg to about 300 mg, about 300 mg to about 325 mg, about 325 mg to about 350 mg, about 350 mg to about 375 mg, about 375 mg to about 400 mg, about 165 mg to about 195 mg, about 195 mg to about 225 mg, about 225 mg to about 255 mg, about 255 mg to about 285 mg, about 285 mg to about 315 mg, about 315 mg, to about 345 mg, or about 345 mg to about 375 mg. In some embodiments, a pharmaceutical composition comprises from about 25 mg to about 200 mg of a plurality of second particulates, including, but not limited to, about 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, or 120 mg. In some embodiments, a pharmaceutical composition comprises from about 25 mg to about 200 mg of a plurality of second particulates, including but not limited to, about 30 mg to about 150 mg, about 30 mg to about 100 mg, about 40 mg to about 100 mg, about 30 mg to about 70 mg, about 47.5 mg to about 52.5 mg, about 25 mg to about 50 mg, about 50 mg to about 75 mg, about 75 mg to about 100 mg, about 100 mg to about 125 mg, about 125 mg to about 150 mg, about 150 mg to about 175 mg, about 175 mg to about 200 mg, about 40 mg to about 70 mg, about 70 mg to about 100 mg, about 100 mg to about 130 mg, about 130 mg to about 160 mg, or about 160 mg to about 190 mg.

In some embodiments, a pharmaceutical composition disclosed herein comprises a plurality of first particulates and a plurality of second particulates. In some embodiments, the plurality of first particulates is present in an amount that ranges from about 150 mg to about 400 mg, including, but not limited to, about 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, or 400 mg. In addition, the plurality of second particulates is present in an amount that ranges from about 25 mg to about 200 mg, including, but not limited to, about 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, or 120 mg. In some embodiments, target and maximum particulate size, including particulate size distribution, is determined through analytical sieving in accordance with USP <786>5 or other appropriately validated methods. Exemplary filters used in particulate size generation include, without limitation, #16, #20, and #30 size mesh screens, corresponding to 1190, 707 and 595 microns in diameter, respectively. In some cases, the particulates of from about 595 microns to about 707 microns, from about 707 microns to about 841 microns, from about 707 microns to about 1190 microns, from about 841 microns to about 1000 microns, or from about 1000 microns to about 1190 microns in diameter. In some embodiments, a pharmaceutical composition disclosed herein comprises a plurality of first particulates comprising one or more first pharmaceutically acceptable excipients and a plurality of second particulates comprising one or more second pharmaceutically excipients. In some embodiments, the one or more first pharmaceutically acceptable excipients and the one or more second pharmaceutically acceptable excipients includes microcrystalline cellulose, hydroxypropyl methylcellulose, croscarmellose sodium, sodium starch glycolate, stearic acid, sodium stearyl fumarate, glyceryl behenate, magnesium stearate, talc, or combinations thereof. In some embodiments, the one or more first pharmaceutically acceptable excipients comprise microcrystalline cellulose, croscarmellose sodium, magnesium stearate, and talc. In some embodiments, the one or more first pharmaceutically acceptable excipients comprise one or more vinyl polymers and a remaining one or more first pharmaceutically acceptable excipients. In some embodiments, the remaining one or more first pharmaceutically acceptable excipients are microcrystalline cellulose, croscarmellose sodium, magnesium stearate, and talc. In some embodiments, the one or more second pharmaceutically acceptable excipients comprise microcrystalline cellulose and croscarmellose sodium. In some embodiments, a pharmaceutical composition disclosed herein comprises a plurality of first particulates comprising a therapeutically effective amount of a triptan and one or more first pharmaceutically acceptable excipients; and a plurality of second particulates comprising a therapeutically effective amount of an antiemetic and one or more second pharmaceutically acceptable excipients; wherein the one or more first pharmaceutically acceptable excipients comprises a vinyl polymer or copolymer. In some embodiments, the triptan is sumatriptan or a pharmaceutically acceptable salt thereof. In some embodiments, the triptan is sumatriptan succinate. In some embodiments, the antiemetic is promethazine or a pharmaceutically acceptable salt thereof. In some embodiments, the antiemetic is promethazine hydrochloride. In some embodiments, the vinyl polymer is polyvinylpyrrolidone. In some embodiments, a pharmaceutical composition comprises a plurality of first particulates comprising a therapeutically effective amount of sumatriptan succinate and one or more first pharmaceutically acceptable excipients; and a plurality of second particulates comprising a therapeutically effective amount of promethazine hydrochloride and one or more second pharmaceutically acceptable excipients; wherein the one or more first pharmaceutically acceptable excipients comprises polyvinylpyrrolidone. In some embodiments, the one or more first pharmaceutically acceptable excipients includes, but is not limited to, microcrystalline cellulose, croscarmellose sodium, magnesium stearate and talc, and the one or more second pharmaceutically acceptable excipients includes, but is not limited to, microcrystalline cellulose and croscarmellose sodium.

In some cases, particulates, e.g., beads or spherules, disclosed herein are coated with a coating material, e.g., a sealant. In some embodiments, the coating material is water soluble. In some embodiments, the coating material comprises a polymer, plasticizer, a pigment, or any combination thereof. In some embodiments, the coating material is a form of a film coating, e.g., a glossy film, a pH independent film coating, an aqueous film coating, a dry powder film coating (e.g., complete dry powder film coating), or any combination thereof. In some embodiments, the coating material is highly adhesive. In some embodiments, the coating material provides low level of water permeation. In some embodiments, the coating material provides oxygen barrier protection. In some embodiments, the coating material allows immediate disintegration for fast release of drug actives. In some embodiments, the coating material is pigmented, clear, or white. In some embodiments, the coating material is clear. Exemplary coating materials include, without limitation, polyvinyl alcohol (PVA), cellulose acetate phthalate (CAP), polyvinyl acetate phthalate (PVAP), methacrylic acid copolymers, cellulose acetate trimellitate (CAT), hydroxypropyl methylcellulose phthalate (HPMCP), hydroxypropyl methylcellulose (HPMC), hydroxy propyl methyl cellulose acetate succinate (hypromellose acetate succinate), shellac, sodium alginate, and zein. In some embodiments, the coating material comprises or is PVA. In some embodiments, the coating material comprises or is HPMC. An exemplary PVA-based coating material includes OPADRY II. In some instances, the coating material is about 1, 2, 3, 4, 5, 6, 7, 8, 9 or 10% of the weight of the particulates, e.g., beads, or spherules. In some instances, the coating material is greater than about 2% of the weight of the particulates, e.g., beads, or spherules.

Dissolution

In some aspects, dissolution rates are measured by a USP Apparatus 1 (Basket Apparatus) at a speed of 100 rpm in a dissolution fluid of 900 mL de-aerated 0.01 N HCl (i.e., pH 2.0) at 37.0±0.5° C. In some instances, dissolution samples are analyzed by HPLC. In some aspects, dissolution of all or less than the entire amount of the active agent. In some embodiments, dissolution of 100% of a pharmaceutically active agent occurs within a prescribed time. In some embodiments, a 5HT_{1B} receptor agonist and an antiemetic both have a dissolution rate of 80% or more within 15 minutes as measured by contact of a pharmaceutical composition disclosed herein with a dissolution fluid in a USP Apparatus 1 (Basket) rotating at about: 50, 60, 70, 80, 90, 100, 110, 120, 130, 140, or 150 rpm, such as 100 rpm. In some embodiments, a 5HT_{1B} receptor agonist or an antiemetic both have a dissolution rate of 80% or more within 30 minutes as measured by contact of a pharmaceutical composition disclosed herein with a dissolution fluid in a USP Apparatus 1 (Basket) rotating at about: 50, 60, 70, 80, 90, 100, 110, 120, 130, 140, or 150 rpm, such as 100 rpm. In some embodiments, a 5HT_{1B} receptor agonist or an antiemetic has a dissolution rate of 80% or more within 15 minutes as measured by contact of a pharmaceutical composition disclosed herein with a dissolution fluid in a USP Apparatus 1 (Basket) rotating at about: 50, 60, 70, 80, 90, 100, 110, 120, 130, 140, or 150 rpm, such as 100 rpm. In some embodiments, a 5HT_{1B} receptor agonist or an antiemetic has a dissolution rate of 80% or more within 30 minutes as measured by contact of a pharmaceutical composition disclosed herein with a dissolution fluid in a USP Apparatus 1 (Basket) rotating at 100 rpm.

In some embodiments, a 5HT_{1B} receptor agonist and an antiemetic both have a dissolution rate of 80% or more within 15 or 30 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, a 5HT_{1B} receptor agonist or an antiemetic has a dissolution rate of 80% or more within 15 minutes or 30 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm.

In some embodiments, dissolution of at least about 60%, 61%, 62%, 63%, 64% or 65% of an antiemetic occurs about 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, dissolution of at least about 80% of an antiemetic occurs about 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, dissolution of at least about 80% of an antiemetic occurs about 30 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, dissolution of at least about 99 or 100% of an antiemetic occurs about 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases the antiemetic is promethazine or a pharmaceutically acceptable salt thereof. In some cases the promethazine salt is promethazine chloride.

In some embodiments, dissolution of at least about 55%, 60%, 65%, 68%, 69%, 70% or 71% of a triptan occurs about 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, dissolution of at least about 80% of a triptan occurs about 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, dissolution of at least about 80% of a triptan occurs about 30 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, dissolution of at least about 99 or 100% of an antiemetic occurs about 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases the triptan is sumatriptan or a pharmaceutically acceptable salt thereof. In some cases, the pharmaceutically acceptable salt of sumatriptan is sumatriptan succinate.

In some embodiments, a pharmaceutical composition comprises an antiemetic and a 5HT1B receptor agonist. In some embodiments, the 5HT_{1B} receptor agonist is a triptan. In some embodiments, the triptan is sumatriptan or a pharmaceutically acceptable salt thereof. In some embodiments, the antiemetic is promethazine or a pharmaceutically acceptable salt thereof. In some cases, the antiemetic has a dissolution rate that is about the same or slower than the dissolution rate of the 5HT_{1B} receptor agonist within about 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has a slower dissolution rate than the dissolution rate of the 5HT_{1B} receptor agonist within about 10 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has a slower dissolution rate than the dissolution rate of the 5HT_{1B} receptor agonist within about 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has a slower dissolution rate than the dissolution rate of the 5HT_{1B} receptor agonist within less than 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has about the dissolution rate as the dissolution rate of the 5HT_{1B} receptor agonist within about 10, 11, 12, 13, 14, 15, 16, 17, 18, 19, 21, 22, 23, 24, 25, 26, 27, 28, 29, or 30 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm.

In some cases, the promethazine or a pharmaceutically acceptable salt thereof and has a dissolution rate that is about the same or slower than the dissolution rate of the 5HT_{1B} receptor agonist within about 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the promethazine or a pharmaceutically acceptable salt thereof has a slower dissolution rate than the dissolution rate of the 5HT_{1B} receptor agonist within about 10 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the promethazine or a pharmaceutically acceptable salt thereof has a slower dissolution rate than the dissolution rate of the 5HT_{1B} receptor agonist within about 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the promethazine or a pharmaceutically acceptable salt thereof has a slower dissolution rate than the dissolution rate of the 5HT_{1B} receptor agonist within less than 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the promethazine or a pharmaceutically acceptable salt thereof has about the dissolution rate as the dissolution rate of the 5HT_{1B} receptor agonist within about 10, 11, 12, 13, 14, 15, 16, 17, 18, 19, 21, 22, 23, 24, 25, 26, 27, 28, 29, or 30 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the pharmaceutically acceptable salt thereof is promethazine hydrochloride.

In some cases, the antiemetic has a dissolution rate that is about the same or slower than the dissolution rate of triptan within about 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has a slower dissolution rate than the dissolution rate of the triptan within about 10 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has a slower dissolution rate than the dissolution rate of the triptan within about 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has a slower dissolution rate than the dissolution rate of the triptan within less than 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has about the dissolution rate as the dissolution rate of the triptan within about 10, 11, 12, 13, 14, 15, 16, 17, 18, 19, 21, 22, 23, 24, 25, 26, 27, 28, 29, or 30 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm.

In some cases, the antiemetic has a dissolution rate that is about the same or slower than the dissolution rate of sumatriptan within about 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has a slower dissolution rate than the dissolution rate of the sumatriptan within about 10 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has a slower dissolution rate than the dissolution rate of the sumatriptan within about 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has a slower dissolution rate than the dissolution rate of the sumatriptan within less than 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the antiemetic has about the dissolution rate as the dissolution rate of the sumatriptan within about 10, 11, 12, 13, 14, 15, 16, 17, 18, 19, 21, 22, 23, 24, 25, 26, 27, 28, 29, or 30 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, the triptan is sumatriptan succinate.

In some cases, the antiemetic dissolves at a faster rate than the triptan. In some cases, the antiemetic is characterized by a greater amount of dissolution after 5 minutes than the triptan following contact with dissolution fluid, and both active ingredients have a similar amount dissolved after 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, (1) about 60% of promethazine hydrochloride is dissolves by 5 minutes following contact with dissolution fluid and about 55% of sumatriptan succinate dissolves by 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm; and (2) about 99% of both active ingredients succinate dissolves by 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm.

In some cases, an antiemetic dissolves at a slower rate than the triptan. In some cases, the antiemetic is characterized by less dissolution after 5 minutes than the triptan, and both active ingredients have a similar amount dissolved by 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some cases, (1) about 60% or about %65 of promethazine hydrochloride is dissolves by 5 minutes following contact with dissolution fluid and about 70% or about 75% of sumatriptan succinate dissolves by 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm; and (2) about 100% of both active ingredients succinate dissolves by 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm.

In some embodiments, dissolution of less than all of the agent occurs in about 1 minute to about 20 minutes (e.g., dissolution of about 55%, about 60%, about 65%, 70%, about 75%, about 80%, about 85%, about 90%, about 91%, about 92%, about 93%, about 94%, about 95%, about 96%, about 97%, about 98%, about 99%, about 99.5% or 99.9% of an agent). Methods for measuring dissolution profiles are known. An example of a method to measure dissolution profiles is provided at Example 4. In some embodiments, about 10% to about 100% of a pharmaceutically active agent achieves dissolution from a plurality of first particulates at about 1 minute to about 60 minutes following contact with a dissolution fluid, such as the dissolution fluid described in Example 4. In some embodiments about 100% of a pharmaceutically active agent achieves dissolution from a plurality of first particulates at about 15, 16, 17, 18, 19 or 20 minutes following contact with a dissolution fluid. In some embodiments, about 10% to about 100% of a pharmaceutically active agent achieves dissolution from a plurality of second particulates at about 1 minute to about 60 minutes following contact with a dissolution fluid. In some embodiments a pharmaceutical composition comprises a plurality of particulates comprising an antiemetic and about 100% of the antiemetic dissolves after about 1 minute to about 60 minutes following contact with a dissolution fluid. In some embodiments, the antiemetic is promethazine or a pharmaceutically acceptable salt thereof. In some embodiments, the antiemetic is promethazine hydrochloride. In some embodiments, a pharmaceutical composition comprises a plurality of particulates comprising a triptan and about 80% of the triptan dissolves after about 15 minutes following contact with a dissolution fluid. In some embodiments, about 100% of the triptan dissolves about 15 or 16 or 17 or 18 or 19 or 20 minutes following contact with a dissolution fluid. In some embodiments, the triptan is sumatriptan or a pharmaceutically acceptable salt thereof. In some embodiments, the triptan is sumatriptan succinate. In some embodiments, a pharmaceutical composition is capable of providing an effective plasma concentration of an antiemetic in about 1 minute to about 60 minutes after administration to a subject. In some embodiments, the pharmaceutical composition is capable providing an effective plasma concentration of promethazine or a pharmaceutically acceptable salt thereof in about 1 minute to about 60 minutes after administration to a subject.

In some aspects, the present disclosure provides for a pharmaceutical composition comprising: a plurality of first particulates comprising a therapeutically effective amount of a triptan or a pharmaceutically acceptable salt thereof and one or more first pharmaceutically acceptable excipients; and a plurality of second particulates comprising a therapeutically effective amount of an antiemetic or a pharmaceutically acceptable salt thereof and one or more second pharmaceutically acceptable excipients, wherein the antiemetic is released faster than the triptan following contact of the pharmaceutical composition with a dissolution fluid. In some embodiments, about 40-95%, for example about: 60-95%, 60-90%, 60-80%, 60-70%, 40%-95%, 40-90%, 40-80%, 40-70%, 50%-95%, 50-90%, 50-80%, 50-70%, 55-65%, 55-70%, 55-80%, 55-90%, or 55-95% of the antiemetic is released within about 5-20 minutes, e.g., about 5-10 minutes or about 5-15 minutes, following contact of the pharmaceutical composition with a dissolution fluid and wherein about 30-90%, for example about: 55-90%, 55-80%, 55-70%, 55-60%, 50-90%, 50-80%, 50-70%, 50-60%, 40-90%, 40-80%, 40-70%, 40-60%, 30-90%, 30-80%, 30-70%, or 30-60% of the triptan is released within about 5-20 minutes, e.g., about 5-10 minutes or about 5-15 minutes, following contact of the pharmaceutical composition with the dissolution fluid. In some embodiments, about: 60%, 95%, 90%, 85%, 80%, 75%, 70%, 65%, 55%, 50%, 45%, or 40% of the antiemetic is released within about 5-10 minutes, e.g., about: 5, 6, 7, 8, 9, or 10 minutes, following contact of the pharmaceutical composition with a dissolution fluid and wherein about: 55%, 90%, 85%, 80%, 75%, 70%, 65%, 60%, 50%, 45%, 40%, 35%, or 30% of the triptan is released within about 5-10 minutes, e.g., about: 5, 6, 7, 8, 9, or 10 minutes, following contact of the pharmaceutical composition with the dissolution fluid. In some embodiments, about: 90-95%, 90-100%, 85-95%, 80-95%, 75%-95%, 70-95%, 65-95%, 60-95%, 50-95%, 45-95%, 40-95%, 85-100%, 80-100%, 75%-100%, 70-100%, 65-100%, 60-100%, 50-100%, 45-100%, 40-100% of the antiemetic is released within about 5-20 minutes, e.g., about: 10, 5, 6, 7, 8, 9, 11, 12, 13, 14, 15, 16, 17, 18, 19, or 20 minutes following contact of the pharmaceutical composition with a dissolution fluid and wherein about: 85-90%, 85-95%, 80-90%, 75%-90%, 70-90%, 65-90%, 60-90%, 50-90%, 45-90%, 40-90%, 35-90%, 30-90%, 80-95%, 75%-95%, 70-95%, 65-95%, 60-95%, 50-95%, 45-95%, 40-95%, 35-95%, or 30-95%, of the triptan is released within about 5-20 minutes, e.g., about: 10, 5, 6, 7, 8, 9, 11, 12, 13, 14, 15, 16, 17, 18, 19, or 20 minutes following contact of the pharmaceutical composition with the dissolution fluid.

In some embodiments, dissolution of an active agent disclosed herein (e.g., triptan, antiemetic) is released in a rate of greater than 80% at 15 minutes. In some embodiments, dissolution of an active agent disclosed herein (e.g., triptan, antiemetic) is released in a rate of greater than 80% at 30 minutes. In some embodiments, at least about 55% of triptan is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 60% of triptan is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 65% of triptan is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 70% of triptan is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 75% of triptan is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 80-85% of triptan is released within 10 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 90% of triptan is released within 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 99% of triptan is released within 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm.

In some embodiments, at least about 55% of triptan succinate is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 60% of triptan succinate is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 65% of triptan succinate is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 70% of triptan succinate is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 75% of triptan succinate is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 80-85% of triptan succinate is released within 10 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 90% of triptan succinate is released within 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 99% of triptan succinate is released within 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm.

In some embodiments, at least about 55% of sumatriptan succinate is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 60% of sumatriptan succinate is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 65% of sumatriptan succinate is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 70% of sumatriptan succinate is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 75% of sumatriptan succinate is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 80-85% of sumatriptan succinate is released within 10 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 90% of sumatriptan succinate is released within 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 99% of sumatriptan succinate is released within 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm.

In some embodiments, at least about 60% of antiemetic is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 65% of antiemetic is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 70% of antiemetic is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 90-95% % of antiemetic is released within 10 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 99% of antiemetic is released within 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 60% of Promethazine HCl is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 65% of Promethazine HCl is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 70% of Promethazine HCl is released within 5 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 90-95% % of Promethazine HCl is released within 10 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm. In some embodiments, at least about 99% of Promethazine HCl is released within 15 minutes, e.g., as measured by contact of a pharmaceutical composition disclosed herein with a dissolution fluid in a USP Apparatus 1 (Basket), e.g., rotating at about: 50, 60, 70, 80, 90, 100, 110, 120, 130, 140, or 150 rpm, such as 100 rpm.

In some embodiments, the weight ratio of the plurality of the first particulates to the plurality of the second particulates is of from about 3:1 to about 5:1. In some embodiments, a pharmaceutical composition disclosed herein is a capsule, comprising: a capsule layer; a plurality of first particulates, wherein each of the first particulates comprises sumatriptan or a pharmaceutically acceptable salt thereof and one or more first pharmaceutically acceptable excipients, wherein the plurality of the first particulates is surrounded by the capsule layer, and wherein a diameter of each of the first particulates is of from about 595 microns to about 1190 microns; and a plurality of second particulates, wherein each of the second particulates comprises promethazine or a pharmaceutically acceptable salt thereof and one or more second pharmaceutically acceptable excipients, wherein the plurality of the second particulates is surrounded by the capsule layer, and wherein a diameter of each of the second particulates is of from about 595 microns to about 1190 microns, wherein the weight ratio of the plurality of the first particulates to the plurality of the second particulates is of from about 3:1 to about 5:1.

In some embodiments, a pharmaceutical composition disclosed herein is stable for at least about: 30 days, 60 days, 90 days, 6 months, 1 year, 18 months, 2 years, 3 years, 4 years, or 5 years, for example about 80%-100% such as about: 80%, 90%, 95%, or 100% of each active pharmaceutical agent in the pharmaceutical composition is stable, e.g., as measured by High Performance Liquid Chromatography (HPLC) such as the HPLC method in Example 5. In some embodiments, about 80%-100% (e.g., about: 90%-100% or 95-100%) of a 5HT1B receptor agonist (e.g., triptan such as sumatriptan) or a pharmaceutically acceptable salt thereof (e.g., sumatriptan succinate) in a pharmaceutical composition disclosed herein is stable for at least about: 30, 60, 90, 180, 360, 540, or 720 days, for example greater than 90 days, which can be measured by HPLC such as the method in Example 5. In some embodiments, about: 80%, 85%, 90%, 95%, or 100% (e.g., about 95%) of the 5HT1B receptor agonist (e.g., triptan such as sumatriptan) or the pharmaceutically acceptable salt thereof (e.g., sumatriptan succinate) is stable for 30 days or more, which can be measured by HPLC such as the method in Example 5. In some embodiments, about 80%-100% (e.g., about: 90%-100% or 95-100%) of an antiemetic (e.g. promethazine or a pharmaceutically acceptable salt thereof such as promethazine hydrochloride) in a pharmaceutical composition disclosed herein is stable for at least about: 30, 60, 90, 180, 360, 540, or 720 days, for example greater than 90 days, which can be measured by HPLC such as the method in Example 5. In some embodiments, about: 80%, 85%, 90%, 95%, or 100% (e.g., about 100%) of the antiemetic (e.g. promethazine or a pharmaceutically acceptable salt thereof such as promethazine hydrochloride) is stable for 30 days or more, which can be measured by HPLC such as the method in Example 5.

Plasma Concentration

In some embodiments, a dosage form of a pharmaceutical composition disclosed herein provides an effective plasma concentration of an antiemetic at from about 1 minutes to about 20 minutes after administration, such as about 1 min, 2 min, 3 min, 4, min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23, min, 24 min, 25 min. In some embodiments, the release occurs at substantially faster rates as compared with release rates for the triptans. Therefore, in some embodiments, after administration to a subject, an antiemetic is released or an effective plasma concentration of an antiemetic is achieved before release of a triptan.

In some embodiments, a dosage form of a pharmaceutical composition provides an effective plasma concentration of a triptan at from about 20 minutes to about 24 hours after administration, such as about 20 min, 30 min, 40 min, 50 min, 1 hr, 1.2 hrs, 1.4 hrs, 1.6 hrs, 1.8 hrs, 2 hrs, 2.2 hrs, 2.4 hrs, 2.6 hrs, 2.8 hrs, 3 hrs, 3.2 hrs, 3.4 hrs, 3.6 hrs, 3.8 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, or 24 hrs following administration. In some embodiments, the triptan is present in an effective plasma concentration in a subject from about 1 hour to about 24 hours or from about 1 day to about 30 days, including, but not limited to, 1, 2, 3, 4, 5, 6, 7, 8, 9, 10, 11, 12, 13, 14, 15, 16, 17, 18, 19, 20, 21, 22, 23, 24, 25, 26, 27, 18, 29 or 30 days.

In some embodiments, a pharmaceutical composition comprises a therapeutically effective amount of each of a triptan and an antiemetic and a polymer, wherein the pharmaceutical composition is capable of providing an effective plasma concentration of the antiemetic prior to an effective plasma concentration of the triptan, post oral administration. In some subjects, tolerance to triptans develops with continued use. In some embodiments, adjustments are made to the amounts or time-release characteristics of one or more pharmaceutically active agents of a pharmaceutical composition, such as a pharmaceutical composition comprising a therapeutically effective amount of each of a triptan and an antiemetic. In some embodiments, the adjustments provide pain relief to a subject with tolerance to triptans. In some embodiments the amount of the triptan is increased in the pharmaceutical composition. In some embodiments the time release characteristics of the triptan are be adjusted by adjusting the amount of a polymer, such as a vinyl polymer or vinyl copolymer, in the pharmaceutical composition. In some embodiments, the polymer which is adjusted is a vinyl polymer, such as polyvinylpyrrolidone, or a vinyl copolymer, such as a polyvinylpyrrolidone/vinyl acetate copolymer. In some embodiments, the pain which is relieved by the adjustments is associated with headache. In some embodiments, the headache is a migraine headache or a cluster headache.

### Methods of Treatment

In some aspects, a method is provided for treating pain, comprising administering to a subject in need thereof a pharmaceutical composition comprising a therapeutically effective amount of each of a triptan and an antiemetic. In some embodiments, a method is provided for treating pain, comprising administering to a subject in need thereof an effective amount of a pharmaceutical composition described herein comprising a polymer or copolymer and a therapeutically effective amount of each of a triptan and an antiemetic.

In some aspects, a method is provided for treating pain, comprising administering to a subject in need a pharmaceutical composition that includes a plurality of first particulates comprising a therapeutically effective amount of a triptan or a pharmaceutically acceptable salt thereof and one or more first pharmaceutically acceptable excipients; and a plurality of second particulates comprising a therapeutically effective amount of an antiemetic or a pharmaceutically acceptable salt thereof and one or more second pharmaceutically acceptable excipients, wherein the one or more first pharmaceutically acceptable excipients comprises a vinyl polymer or a vinyl copolymer. In some embodiments, the plurality of first particulates and the plurality of second particulates are encapsulated into discrete units. In some embodiments, the discrete units are capsules or packets. In some embodiments, a method is provided for treating pain, comprising administering the capsule or the packet containing a plurality of particulates as described herein. In some embodiments, a method of treating pain includes breaking the capsule or the packet to sprinkle the plurality of particulates on food or soft foods and swallowed without chewing. In some embodiments, the plurality of particulates is administered through an enteral feeding tube. In some embodiments the pain is associated with a headache, such as a chronic headache, cluster headache or a migraine headache. In one embodiment the migraine headache occurs with aura. In some embodiments, the migraine headache is accompanied by symptoms, including, but not limited to vomiting, nausea, photophobia, phonophobia, or osmophobia.

In some embodiments, the photophobia is characterized by light sensitivity or light hypersensitivity. In some cases, the photophobia is caused by acute iritis or uveitis (inflammation inside eye), burns to the eye, corneal abrasion, corneal ulcer, drug side effects, excessive wearing of contact lenses, or wearing badly-fitted contact lenses, eye disease, injury, or infection (such as chalazion, episcleritis, glaucoma), eye testing when the eyes have been dilated, meningitis, migraine headache, or recovery from eye surgery. In some cases, the photophobia is associated with a migraine. In some cases, the photophobia is associated with nausea and vomiting. In some cases, the photophobia is associated with nausea or vomiting.

In some embodiments, a pharmaceutical composition defined herein is for the reduction of ocular pain, itching, burning, and/or stinging, and/or photophobia, following a surgery or postoperative inflammation. In some embodiments, a pharmaceutical composition defined herein is given at the time of pupil dilation. In some embodiments, a pharmaceutical composition disclosed herein is for use in treatment of a photophobia wherein the treatment is prophylactic. In instances cases, a pharmaceutical composition disclosed herein is for use in treatment of a photophobia wherein the treatment is preventative. In some cases, preventative treatment is to decrease migraine frequency. In some embodiments, a pharmaceutical composition disclosed herein is for use in treatment of a photophobia wherein the treatment is preemptive. In some cases, preemptive treatment is used when a photophobia trigger is time-limited or predictable. In some embodiments, a pharmaceutical composition disclosed herein is for use in treatment of a photophobia wherein the treatment is acute. In some cases, treatment is to stop or prevent progression of a photophobia. In some cases, acute treatment is initiated during an attack to relieve pain. In some cases, a pharmaceutical composition disclosed here is used for preventive, acute, and/or preemptive treatment for photophobia.

In some embodiments, a pharmaceutical composition disclosed herein is for use in treatment of a headache wherein the treatment is prophylactic. In instances cases, a pharmaceutical composition disclosed herein is for use in treatment of a headache wherein the treatment is preventative. In some cases, preventative treatment is to decrease migraine frequency. In some embodiments, a pharmaceutical composition disclosed herein is for use in treatment of a headache wherein the treatment is preemptive. In some cases, preemptive treatment is used when a headache trigger is time-limited or predictable. In some embodiments, a pharmaceutical composition disclosed herein is for use in treatment of a headache wherein the treatment is acute. In some cases, treatment is to stop or prevent progression of a migraine. In some cases, acute treatment is initiated during an attack to relieve pain. In some cases, a pharmaceutical composition disclosed here is used for preventive, acute, and/or preemptive treatment for a headache.

In some embodiments, a pharmaceutical composition disclosed herein is used for treatment of chronic migraine headache. In some embodiments, a pharmaceutical composition disclosed herein is for use in treatment of a migraine headache wherein the treatment is prophylactic. In some embodiments, a pharmaceutical composition disclosed herein is for use in treatment of a migraine headache wherein the treatment is of an acute migraine headache. In some embodiments, a pharmaceutical composition disclosed herein is for use in treatment of a migraine wherein the treatment is of a chronic migraine headache. In some embodiments, a pharmaceutical composition disclosed herein is for use in treatment of a migraine headache with an aura. In some embodiments, a pharmaceutical composition disclosed herein is for use in treatment of a migraine headache without an aura. In some embodiments, a pharmaceutical composition disclosed herein is for use in treatment of a cluster headache. In some embodiments, a pharmaceutical composition disclosed herein is for use in treatment of nausea or vomiting. In some embodiments, a pharmaceutical composition disclosed herein is for use in treatment of nausea and vomiting. In some embodiments, a pharmaceutical composition disclosed herein is for use in treatment of nausea associated with a headache or vomiting associated with a headache. In some embodiments, a pharmaceutical composition disclosed herein is for use in treatment of nausea associated with a headache and vomiting associated with a headache. In some embodiments, a pharmaceutical composition disclosed herein is for use in treatment of nausea associated with a migraine headache or vomiting associated with a migraine headache. In some embodiments, a pharmaceutical composition disclosed herein is for use in treatment of nausea associated with a migraine headache and vomiting associated with a migraine headache.

In some embodiments, a pharmaceutical composition disclosed herein (e.g., capsule) does not completely disintegrate in mouth within about: 1, 2, 3, 4, 5, 6, 7, 8, 9, 10, 11, 12, 13, 14, 15, 16, 17, 18, 19 or 20 minutes. In some embodiments, a pharmaceutical composition disclosed herein is not a film. In some embodiments, a pharmaceutical composition disclosed herein is not for buccal administration. In some embodiments, a pharmaceutical composition disclosed herein (e.g., capsule) dissolves in stomach or intestine.

In some embodiments, the subject is a mammal, e.g., a human, mouse, rat, guinea pig, dog, cat, horse, cow, pig, or non-human primate, such as a monkey, chimpanzee or baboon. In some embodiments, the subject is a human. In some embodiments, the subject administered a pharmaceutical composition as described herein is about 55 years of age or older, about 60 years of age or older, about 65 years of age or older, or about 70 years of age or older. In some embodiments, the subject administered a pharmaceutical composition described herein is 18 years of age or older. In some embodiments, the subject is between 35 and 45 years of age. In some embodiments, the subject administered a pharmaceutical composition described herein has a history of headaches. In some embodiments, the subject administered a pharmaceutical composition described herein has a history of migraines.

In some embodiments, the pharmaceutical composition described herein is administered to the subject (e.g., a patient) at the time of onset of the migraine headache as needed by the subject (e.g., a patient) or as determined and instructed by the physician. In some embodiments, the subject administered a pharmaceutical composition described herein suffers from adverse effects associated with triptan administration. Examples of adverse effects include nausea and/or vomiting, e.g., associated with a migraine. In some embodiments, the pharmaceutical composition described herein reduces or prevents unwanted side effects associated with injectable or tablet triptan therapy, including, flushing, sweating, vertigo, fatigue, tingling, drowsiness, dizziness, dry mouth, heartburn, abdominal pain, abdominal cramps, weakness, feeling of warmth or coldness, bitter taste from tablets and nasal sprays, and local burning from injection site.

In some embodiments, a pharmaceutical composition described herein is administered to a subject at about every 12 to about 24 hours, about every 12 hours, or about every 24 hours. In some embodiments, a pharmaceutical composition described herein is administered to a subject at about every 8 to about every 12 hours. In some embodiments, a pharmaceutical composition described herein is administered once, twice or three times daily. In some embodiments, a pharmaceutical composition described herein is administered no more than twice daily. In some embodiments, a second dose of a pharmaceutical composition disclosed herein is administered after response to a first dose in a subject. In some embodiments, doses after a first dose of a pharmaceutical composition described herein are separated by at least 2 hours. In some embodiments, the maximum dose of a pharmaceutical composition described herein over a 24 hour period does not exceed 200 mg. In some embodiments, a maximum single dose of a pharmaceutical composition described herein dose does not exceed 50 mg in a subject with mild to moderate hepatic impairment.

In some embodiments, a pharmaceutical composition described herein comprising sumatriptan succinate and promethazine hydrochloride is administered to a subject at about every 12 to about 24 hours, about every 12 hours, or about every 24 hours. In some embodiments, a pharmaceutical composition described herein comprising sumatriptan succinate and promethazine hydrochloride is administered to a subject at about every 8 to about every 12 hours. In some embodiments, a pharmaceutical composition described herein comprising sumatriptan succinate and promethazine hydrochloride is administered once, twice or three times daily. In some embodiments, a pharmaceutical composition described herein comprising sumatriptan succinate and promethazine hydrochloride is administered no more than twice daily. In some embodiments, a second dose of a pharmaceutical composition disclosed herein comprising sumatriptan succinate and promethazine hydrochloride is administered after response to a first dose in a subject. In some embodiments, doses after a first dose are separated by at least 2 hours. In some embodiments, the maximum dose of a pharmaceutical composition disclosed herein comprising sumatriptan succinate and promethazine hydrochloride over a 24 hour period does not exceed 200 mg. In some embodiments, a maximum single dose of a pharmaceutical composition disclosed herein comprising sumatriptan succinate and promethazine hydrochloride does not exceed 50 mg in a subject with mild to moderate hepatic impairment. In some embodiments, the frequency of dosing is determined or assessed by a professional assessing the subject, the severity of the condition and expected duration of therapy.

In some aspects, a method is provided for treating pain comprises administering to a subject in need thereof a pharmaceutical composition comprising a therapeutically effective amount of a triptan; an antiemetic; and a vinyl polymer. In some embodiments, the pain is a headache. In some embodiments, the headache is a migraine headache. In some embodiments the headache is a cluster headache. In some embodiments, the method is also useful for treating photophobia. In some embodiments, the photophobia is associated with migraine headache. In some embodiments, a method for treating headache comprises: administering to a subject in need thereof a pharmaceutical composition comprising a therapeutically effective amount of sumatriptan or a pharmaceutically acceptable salt thereof; promethazine or a pharmaceutically acceptable salt thereof; and a vinyl polymer. In some embodiments the vinyl polymer is polyvinylpyrrolidone. In some embodiments the vinyl polymer is polyvinylpolypyrrolidone. In some embodiments, a method for treating headache comprises administering to a subject in need thereof a pharmaceutical composition comprising: a therapeutically effective amount of sumatriptan or a pharmaceutically acceptable salt thereof; promethazine or a pharmaceutically acceptable salt thereof; and a vinyl copolymer. In one embodiment the vinyl copolymer is a polyvinylpyrrolidone/vinyl acetate copolymer or a polyvinylpyrrolidone/polyvinyl acetate copolymer. In some embodiments the vinyl copolymer is a vinylpolypyrrolidone/vinyl acetate copolymer. In some embodiments, a method for treating headache comprises administering to a subject in need thereof a pharmaceutical composition comprising: a plurality of first particulates comprising a therapeutically effective amount of a triptan and one or more first pharmaceutically acceptable excipients; and a plurality of second particulates comprising a therapeutically effective amount of an antiemetic and one or more second pharmaceutically acceptable excipients; wherein the one or more first pharmaceutically acceptable excipients comprises a vinyl polymer or a vinyl copolymer. In one embodiment the headache is a migraine headache. In some embodiments the headache is a cluster headache. In some embodiments, a method for treating headache comprises administering to a subject in need thereof a pharmaceutical composition comprising: a plurality of first particulates comprising a therapeutically effective amount of sumatriptan or a pharmaceutically acceptable salt thereof and one or more first pharmaceutically acceptable excipients; and a plurality of second particulates comprising a therapeutically effective amount of promethazine or a pharmaceutically acceptable salt thereof and one or more second pharmaceutically acceptable excipients; wherein the one or more first pharmaceutically acceptable excipients comprises polyvinylpyrrolidone. In some embodiments, a method for treating headache comprises administering to a subject in need thereof a pharmaceutical composition comprising: a plurality of first particulates comprising a therapeutically effective amount of sumatriptan succinate, polyvinylpyrrolidone, microcrystalline cellulose, croscarmellose sodium, magnesium stearate, and talc; and a plurality of second particulates comprising a therapeutically effective amount of promethazine hydrochloride, microcrystalline cellulose, and croscarmellose sodium. In some embodiments, a method for treating headache comprises administering to a subject in need thereof a pharmaceutical composition comprising: a plurality of first particulates comprising from about 84 mg to about 126 mg of sumatriptan succinate, from about 1.05 mg to about 10.5 mg of polyvinylpyrrolidone, from about 42 mg to about 126 mg of microcrystalline cellulose, from about 1.05 mg to about 10.5 mg of croscarmellose sodium, from about 0.525 mg to about 10.5 mg of magnesium stearate, and from about 2.1 mg to about 10.5 mg of talc; and a plurality of second particulates comprising from about 20 mg to about 30 mg of promethazine hydrochloride, from about 10 mg to about 30 mg of microcrystalline cellulose, and from about 0.25 mg to about 2.5 mg of croscarmellose sodium. In some embodiments, a method for treating headache comprises administering to a subject in need thereof a pharmaceutical composition comprising: a plurality of first particulates comprising about 126 mg of sumatriptan succinate, about 4.2 mg of polyvinylpyrrolidone, about 72.45 mg of microcrystalline cellulose, about 4.2 mg of croscarmellose sodium, about 1.05 mg of magnesium stearate, and about 2.1 mg of talc; and a plurality of second particulates comprising about 25 mg of promethazine hydrochloride, about 24 mg of microcrystalline cellulose, and about 1 mg of croscarmellose sodium.

Methods of Manufacture

In some embodiments, a method is provided for manufacturing a pharmaceutical composition as described herein. In some embodiments, the pharmaceutical composition as described herein is prepared by standard techniques and using standard equipment known to the skilled person. In some embodiments, a plurality of particulates comprising an active pharmaceutical ingredient such as triptan or an antiemetic are prepared by a process method comprising wet granulation, extrusion and spheronization. In some embodiments, a triptan (e.g., sumatriptan or other triptans disclosed herein) or an antiemetic (e.g., promethazine) and one or more second pharmaceutically acceptable excipients are screened through a suitable size mesh screen into a granulator container. In some embodiments, the triptan or the antiemetic and one or more second pharmaceutically acceptable excipients are blended in a high shear granulator at an appropriate speed for an appropriate period of time. In some embodiments, a binder solution is prepared by dissolving a polymer such as polyvinylpyrrolidone in water and mixed for a period of time in a stir assembly.

In some embodiments, granulation is performed according to fixed parameters such as impeller speed, chopper speed and binder solution/water flow rate. In some embodiments, the impeller speed is 300-400 rpm, the chopper speed is 700-750 rpm and the binder solution/water flow rate is 40 g/minute. In some embodiments, the wet mass is loaded onto a multi granulator extruder such as a LCI MG-55 Multi granulator extruder equipped with an appropriate screen size and set at an appropriate speed, for example, at 50 rpm, 60 rpm, or 70 rpm. In some embodiments, extrudes obtained is charged to a spheronizer such as LCI QJ-230T Marumerizer spheronizer equipped with 2 mm cross hatch disc or any other appropriate sized disc. In some embodiments, the speed of the spheronizer is between 1100-1700 rpm. In some embodiments, thespheronization time is 10 seconds, 20 seconds, 30 seconds, 40 seconds, 50 seconds, 60 seconds, 70 seconds, 80 seconds, 90 seconds, 100 seconds, 110 seconds or 120 seconds. In some embodiments, the particulates, e.g., spherules/beads, obtained are transferred to a vector fluid bed dryer. In some embodiments, the dryer presets drying parameters such as, but not limited to, inlet temperature of between 55-65° C. or 70° C., outlet temperature of between 20-30° C. or 30-40° C., product temperature of between 20-45° C. or 21-42° C., total time of 45-75 minutes, fan at 180-740 lpm (liters per minute). In some embodiments, loss on drying (LOD) values following the drying step is between 1.5-3%. In some embodiments, the particulates, e.g., spherules/beads, are sifted through a nest of screens of size #16 to #30 to further determine particle size range. In some embodiments, the plurality of particulates is mixed with talc or a coating material. In one example, the mixing is performed by inversion or swirling. In some embodiments, the plurality of particulates comprising an active pharmaceutical ingredient such as triptan or an antiemetic and a pharmaceutically acceptable excipient are weighed and combined in a discrete unit at predetermined weight ratios.

In some aspects, a method is provided for manufacturing a pharmaceutical composition that comprises: producing a plurality of first particulates by performing wet granulation on a mixture composed of a triptan or a pharmaceutically acceptable salt thereof and one or more pharmaceutically acceptable excipients, adding a binder solution containing at least one polymer to the mixture at an appropriate time and in a sufficient quantity to form granules, forming extrudes of a wet mass containing the mixture and binder solution, and subjecting the extrudes to spheronization parameters sufficient in disc diameter, speed, and time to produce particulates (e.g., spherules or beads); and producing a plurality of second particulates by performing wet granulation on a mixture composed of an antiemetic or a pharmaceutically acceptable salt thereof and one or more pharmaceutically acceptable excipients, forming extrudes of a wet mass containing the mixture and binder solution, and subjecting the extrudes to spheronization parameters sufficient in disc diameter, speed, and time to produce particulates (e.g., spherules or beads). In some embodiments, a pharmaceutical composition is provided in the form of a capsule, wherein the capsule comprises a plurality of first particulates and a plurality of second particulates, wherein each particulate comprises one or more pharmaceutically active agents disclosed herein. In some embodiments, the capsule comprises a plurality of first particulates comprising sumatriptan succinate and a plurality of second particulates comprising promethazine hydrochloride.

Stability

In some aspects, a pharmaceutical composition disclosed herein is stable for at least about: 30 days, 60 days, 90 days, 6 months, 1 year, 18 months, 2 years, 3 years, 4 years, or 5 years, for example about 80%-100% such as about: 80%, 90%, 95%, or 100% of each active pharmaceutical agent in the pharmaceutical composition is stable, e.g., as measured by High Performance Liquid Chromatography (HPLC) such as the HPLC method in Example 5. In some embodiments, about 80%-100% (e.g., about: 90%-100% or 95-100%) of a 5HT_{1B} receptor agonist (e.g., triptan such as sumatriptan) or a pharmaceutically acceptable salt thereof (e.g., sumatriptan succinate) in a pharmaceutical composition disclosed herein is stable for at least about: 30, 60, 90, 180, 360, 540, or 720 days, for example greater than 90 days, which can be measured by HPLC such as the method in Example 5. In some embodiments, about: 80%, 85%, 90%, 95%, or 100% (e.g., about 95%) of the 5HT_{1B} receptor agonist (e.g., triptan such as sumatriptan) or the pharmaceutically acceptable salt thereof (e.g., sumatriptan succinate) is stable for 30 days or more, which can be measured by HPLC such as the method in Example 5. In some embodiments, the 5HT_{1B} receptor agonist (e.g., triptan such as sumatriptan) or the pharmaceutically acceptable salt thereof (e.g., sumatriptan succinate) comprises a coating material. In some embodiments, about 80%-100% (e.g., about: 90%-100% or 95-100%) of an antiemetic (e.g. promethazine or a pharmaceutically acceptable salt thereof such as promethazine hydrochloride) in a pharmaceutical composition disclosed herein is stable for at least about: 30, 60, 90, 180, 360, 540, or 720 days, for example greater than 90 days, which can be measured by HPLC such as the method in Example 5. In some embodiments, about: 80%, 85%, 90%, 95%, or 100% (e.g., about 100%) of the antiemetic (e.g. promethazine or a pharmaceutically acceptable salt thereof such as promethazine hydrochloride) is stable for 30 days or more, which can be measured by HPLC such as the method in Example 5. In some embodiments, the antiemetic (e.g. promethazine or a pharmaceutically acceptable salt thereof such as promethazine hydrochloride) comprises a coating material. In some embodiments, the coating material in the 5HT_{1B} receptor agonist and/or antiemetic comprises polyvinyl alcohol, cellulose acetate phthalate, polyvinyl acetate phthalate, methacrylic acid copolymer, cellulose acetate trimellitate, hydroxypropyl methylcellulose phthalate, hydroxypropyl methylcellulose, hydroxy propyl methyl cellulose acetate succinate, shellac, sodium alginate, or zein, for example polyvinyl alcohol. In some embodiments, the coating material in the 5HT_{1B} receptor agonist and/or antiemetic is polyvinyl alcohol.

In some embodiments, the weight ratio of the plurality of the first particulates to the plurality of the second particulates is of from about 3:1 to about 5:1. In some embodiments, a pharmaceutical composition disclosed herein is a capsule, comprising: a capsule layer; a plurality of first particulates, wherein each of the first particulates comprises sumatriptan or a pharmaceutically acceptable salt thereof and one or more first pharmaceutically acceptable excipients, wherein the plurality of the first particulates is surrounded by the capsule layer, and wherein a diameter of each of the first particulates is of from about 595 microns to about 1190 microns; and a plurality of second particulates, wherein each of the second particulates comprises promethazine or a pharmaceutically acceptable salt thereof and one or more second pharmaceutically acceptable excipients, wherein the plurality of the second particulates is surrounded by the capsule layer, and wherein a diameter of each of the second particulates is of from about 595 microns to about 1190 microns, wherein the weight ratio of the plurality of the first particulates to the plurality of the second particulates is of from about 3:1 to about 5:1.

In some embodiments, a pharmaceutical composition disclosed herein is a fast release pharmaceutical composition, wherein at least about 80% of both the sumatriptan or a pharmaceutically acceptable salt thereof and the promethazine or a pharmaceutically acceptable salt thereof are released within about 15 minutes as measured by contact of the pharmaceutical composition with a dissolution fluid in a USP Apparatus 1 (Basket) rotating at 100 rpm. In some embodiments, a pharmaceutical composition disclosed herein is a fast release pharmaceutical composition, comprising: a plurality of first particulates, wherein each of the first particulates comprises sumatriptan or a pharmaceutically acceptable salt thereof; and a plurality of second particulates, wherein each of the second particulates comprises promethazine or a pharmaceutically acceptable salt thereof, wherein at least about 80% of both the sumatriptan or a pharmaceutically acceptable salt thereof and the promethazine or a pharmaceutically acceptable salt thereof are released within about 15 minutes as measured by contact of the pharmaceutical composition with a dissolution fluid in a USP Apparatus 1 (Basket) rotating at 100 rpm.

### Examples

The following examples are offered by way of illustration and not by way of limitation.

### Example 1. Preparation of Formulation I

Sumatriptan particulates and promethazine particulates were generated, and then encapsulated together in a capsule. Formulation of sumatriptan 90 mg particulates was performed as described below. A list of ingredients is provided in Table 1. Each API was spheronized into separate particulates and filled in a capsule in the appropriate ratio.

TABLE 1
Formulation of Sumatriptan particulates

| | | | Batch |
|---|---|---|---|
| Ingredient | Percent w/w | mg/dose | Quantity (g) |
| Sumatriptan succinate equivalent to | 60.00 | 126.00 | 180.00 |
| 90 mg sumatriptan | | | |
| Microcrystalline cellulose, NF, | 34.50 | 72.45 | 103.50 |
| Ph. Eur., JP (Avicel PH101) | | | |
| Polyvinylpyrrolidone (Plasdone | 2.00 | 4.20 | 6.00 |
| K29/32) | | | |
| Croscarmellose sodium, NF, Ph. | 2.00 | 4.20 | 6.00 |
| Eur., JP (Ac-Di-Sol) | | | |
| Magnesium stearate, NF Kosher | 0.50 | 1.05 | 1.50 |
| Passover (Hyqual 5712) | | | |
| Talc | 1.00 | 2.10 | 3.00 |
| Purified Water* | qs | qs | qs |
| Total | | 210.00 | 300.00 |

Sumatriptan, microcrystalline cellulose, croscarmellose sodium and magnesium stearate were screened through #20 mesh screen to the high shear mixer granulator bowl. The ingredients were blended in the high shear granulator at 250 rpm for 5 minutes and LOD of dry mixture was measured (2.303%). Binder solution was prepared by dissolving polyvinylpyrrolidone (6 g) in purified water (24 g) and mixed for 45 minutes using an appropriate stir assembly. Granulation was performed using following parameters: granulator bowl size of 1 L, impeller speed of 300 rpm, chopper speed of 700 rpm, and binder solution/water flow rate of 40 g/minute.

A total of 30 g binder solution and 128 g of water was added in the granulation bowl and mixed for 3 min 35 sec. Wet mass mixing was performed for 2 minutes after addition of water using 300 rpm impeller at 700 rpm chopper speed. The wet mass was loaded on the LCI MG-55 Multi granulator extruder equipped with 1.0 mm screen size at 65 rpm speed. The extrudes were obtained and charged to the LCI QJ-230T Marumerizer spheronizer equipped with 2 mm cross hatch disc. The following parameters were used for spheronization: 2 mm disc, 1200 rpm speed, and 30 second spheronization time.

The particulates obtained were then transferred to vector fluid bed dryer for drying in 2 sub lots. Drying parameters were as follows: inlet temperature of 70° C., outline temperature of 20-30° C., Fan (%) of 180-740 Ipm, total time of 45 minutes, LOD obtained after drying for sublot 1=1.834%, LOC obtained after drying for sublot 2: 1.979%. The particulates were then sifted through a nest of screens sizes of #16 and #30 to determine particle size range.

Formulation of promethazine HCl 25 mg particulates was performed as described below. A list of ingredients is provided in Table 2.

TABLE 2
Formulation of Promethazine HCl particulates

| | | | Batch |
|---|---|---|---|
| Ingredient | Percent w/w | mg/dose | Quantity (g) |
| Promethazine hydrochloride | 50.00 | 25.00 | 150.00 |
| Microcrystalline cellulose, NF, Ph. | 48.00 | 24.00 | 144.00 |
| Eur., JP (Avicel PH101) | | | |
| Croscarmellose sodium, NF, Ph. | 2.00 | 1.00 | 6.00 |
| Eur., JP (Ac-Di-Sol) | | | |
| Purified Water* | qs | qs | qs |
| Total | 100.00 | 50 | 300 |

Promethazine, microcrystalline cellulose and croscarmellose sodium were screened through #20 mesh screen to the high shear mixer granulator bowl. The ingredients were blended in the high shear granulator at 250 rpm for 5 minutes and LOD of dry mixture was measured (2.831%). Granulation was performed using purified water. Granulation parameters were as follows: granulator bowl size of 2 L, impeller speed of 400 rpm, chopper speed of 750 rpm, and binder solution/water flow rate of 40 g/minute.

A total of 75 g of water was added in granulation bowl for 1 min 55 sec. A 15 second wet mass mixing was performed after addition of water using 400 rpm impeller and 750 rpm chopper speed. The wet mass was loaded on the LCI MG-55 Multi granulator extruder equipped with 1.0 mm screen size at 65 rpm speed. The extrudes obtained were charged to the LCI QJ-230T Marumerizer spheronizer equipped with 2 mm cross hatch disc. The following parameters were used for spheronization: 2 mm disc, 1600 rpm speed, and 2 minutes spheronization time.

The particulates obtained were transferred to 4 L fluid bed dryer for drying. The drying parameters were as follows: inlet temperature of 555-65° C., outline temperature of 27-40° C., Fan (%) of 45-75 Ipm, total time of 50 minutes, LOD obtained after drying=2.80434%. The particulates obtained from the steps above were sifted through a nest of screens of sizes #16 and #30 to determine particle size range.

Formulation of capsules comprising sumatriptan and promethazine was performed as described Table 3 and detailed below.

TABLE 3
Formulation and Encapsulation - 100 capsules

| | | | | | Batch |
|---|---|---|---|---|---|
| | Manufacturer | Exp | Percent | mg/ | Quantity |
| Ingredient | Lot Number | Date | w/w | capsule | (g) |
| Sumatriptan | Xcelience | NA | 79.81 | 207.90 | 20.79 |
| particulates | N2999-43 | | | | |
| Talc | Imerys | | 0.7981 | 2.08 | 0.208 |
| | H06033 | | | | |
| Total | | | | 298.98 | 21.0 |
| Promethazine | Xcelience | NA | 19.20 | 50.00 | 5.00 |
| particulates | N2999-76 | | | | |
| Talc | Imerys | | 0.192 | 0.5 | 0.05 |
| | H06033 | | | | |
| Total | | | | 50.5 | 5.05 |
| Capsules, | Capsugel | | N/A | 1 capsule | 100 |
| Size 0 | 90177971 | | | | capsules |
| CS white | | | | | |
| opaque | | | | | |
| gelatin capsules | | | | | |

| | | |
|---|---|---|
| Total | 260.48 | 26.05 |

Sumatriptan and talc were manually mixed in an amber glass bottle by inversion/swirling. Promethazine and talc were manually mixed in an amber glass bottle by inversion/swirling. The average weight of 100 empty capsules obtained was 92.85 mg. 210.0 mg (200-220 mg) of sumatriptan particulates and 50.0 mg (47.5-52.5 mg) of the promethazine particulates was manually weighed and filled in each individual capsule. Since the particulates had static, a glass funnel helped for filling. The capsules were packaged in opaque HDPE bottles. Encapsulation was performed under yellow lighting.

### Example 2. Preparation of Formulation II

Sumatriptan particulates and promethazine particulates were generated, and then encapsulated together in a capsule. Formulation of sumatriptan 90 mg coated particulates was performed as described below. A list of ingredients is provided in Table 4. Each API was spheronized into separate particulates.

TABLE 4
Formulation of Sumatriptan Particulates

| | | Batch |
|---|---|---|
| Ingredient | Percent w/w | Quantity (g) |
| Sumatriptan succinate USP | 60.61 | 1827.2 |
| Microcrystalline Cellulose, NF (AVICEL | 34.85 | 1050.7 |
| PH101) | | |
| Croscarmellose Sodium, NF (AC-DI-SOL) | 2.02 | 60.9 |
| Povidone (Plasdone K29/32) | 2.02 | 60.9 |
| Magnesium Stearate, NF | 0.5 | 15.4 |
| (Kosher Passover Hyqual) | | |
| Sterile Water for Irrigation, USP | qs | 1000.0 |
| Total | 100.00 | 3014.9 |

Sumatriptan succinate, microcrystalline cellulose, croscarmellose sodium and magnesium stearate were screened through #20 mesh screen to a high shear mixer granulator bowl. The ingredients were blended in the high shear granulator at about 150 rpm for 5 minutes. Binder solution was prepared by dissolving Povidone (2.02 g) in sterile water (246.3 g) and mixed using an appropriate stir assembly. Granulation was performed using following parameters: impeller speed of 300 rpm, chopper speed of 700 rpm, and binder solution/water flow rate of 80 g/minute.

Binder solution and water were added in the granulation bowl and mixed. The wet mass was loaded on the LCI MG-55 Multi granulator extruder equipped with 1.0 mm screen size at 65 rpm speed. The extrudes were obtained and charged to the LCI QJ-230T Marumerizer spheronizer equipped with 2 mm cross hatch disc. The following parameters were used for spheronization: 2 mm disc, 1200 rpm speed, and 30 second spheronization time.

The particulates obtained were then transferred to vector fluid bed dryer for drying in sub lots as needed. Drying parameters were as follows: inlet temperature of 60° C. Drying was done to a LOD % target of +/−1% of an LOD testing recorded after granulation. The particulates were then sifted through a nest of screens of sizes #16 and #30 to determine particle size range.

Material amounts for coated particulates generated are provided in Table 5. To generate coating solution, sterile water and for irrigation was stirred in a mixer with OPADRY II Complete Film Coating System 85F19250 Clear. After all the OPADRY II Complete Film Coating System 85F19250 Clear had been mixed, the mixer speed was reduced and mixing continued for 45 minutes. A calibrated spray nozzle with a spray rate of 1.0 g/min/kg was used to spray the coating solution on the particulates. The nozzle was adjusted to air to a target of 0.7 psig. Inlet and exhaust fans were used with an inlet air temperature of 60 to 80° C. The coating endpoint was application sufficient fora 2.0% weight gain to the particulates.

TABLE 5
Formulation of Coated Particulates - 2.0% Target Weight Gain

| | | Amount/Coated | |
|---|---|---|---|
| | Concentration | Particulate | Amount/Batch |
| Ingredient | (Percent w/w) | (mg) | (g) |
| Sumatriptan 90 mg, | 98.04 | 207.90 | 3014.9 |
| Particulate | | | |
| OPADRY II Complete | 1.96 | 4.158 | 60.3 |
| Film Coating System | | | |
| 85F19250 Clear | | | |
| Sterile Water for | N/A | N/A | 1447.2 |
| Irrigation, USP | | | |
| Total | 100.0 | 212.06 | 3014.9 |

2.2 Promethazine Particulates and Coated Particulates. Formulation of promethazine HCl 25 mg coated particulates was performed as described below. A list of ingredients is provided in Table 6.

TABLE 6
Formulation of Promethazine Particulates

| | | Batch |
|---|---|---|
| Ingredient | Percent w/w | Quantity (g) |
| Promethazine HCl | 50.0 | 1516.8 |
| Microcrystalline Cellulose, NF (AVICEL | 48.0 | 1456.1 |
| PH101) | | |
| Croscarmellose Sodium, NF (AC-DI-SOL) | 2.0 | 60.7 |
| Sterile Water for Irrigation, USP | qs | 1000.0 |
| Total | 100.00 | 3033.6 |

Promethazine HCl, microcrystalline cellulose and croscarmellose sodium were screened through #20 mesh screen to a high shear mixer granulator bowl. The ingredients were blended in the high shear granulator at about 150 rpm for 5 minutes. 707.8 g of Sterile Water was prepared for irrigation. Granulation was performed using following parameters: impeller speed of 400 rpm, chopper speed of 750 rpm, and binder solution/water flow rate of 70 g/minute. Sterile water was added in the granulation bowl and mixed. The wet mass was loaded on the LCI MG-55 Multi granulator extruder equipped with 1.0 mm screen size at 65 rpm speed. The extrudes were obtained and charged to the LCI QJ-230T Marumerizer spheronizer equipped with 2 mm cross hatch disc. The following parameters were used for spheronization: 2 mm disc, 1600 rpm speed, and 2 minute spheronization time.

The particulates obtained were then transferred to vector fluid bed dryer for drying in sub lots as needed. Drying parameters were as follows: inlet temperature of 60° C. Drying was done to a LOD % target of +/−1% of an LOD testing recorded after granulation. The particulates were then sifted through a nest of screens #16 and #30 to determine particle size range.

Material amounts for coated particulates generated are provided in Table 7. To generate coating solution, sterile water and for irrigation was stirred in a mixer with OPADRY II Complete Film Coating System 85F19250 Clear. After all the OPADRY II Complete Film Coating System 85F19250 Clear had been mixed, the mixer speed was reduced and mixing continued for 45 minutes. A calibrated spray nozzle with a spray rate of 1.7 g/min was used to spray the coating solution on the particulates. The nozzle was adjusted to air to a target of 0.7 psig. Inlet and exhaust fans were used with an inlet air temperature of 60 to 80° C. The coating endpoint was application sufficient fora 2.0% weight gain to the particulates.

TABLE 7
Formulation of Coated Particulates - 2.0% Target Weight Gain

| | | Amount/Coated | |
|---|---|---|---|
| | Concentration | Particulate | Amount/Batch |
| Ingredient | (Percent w/w) | (mg) | (g) |
| Promethazine HCl | 98.04 | 50.0 | 3033.6 |
| 25 mg, Particulate | | | |
| OPADRY II Complete | 1.96 | 1.00 | 60.7 |
| Film Coating System | | | |
| 85F19250 Clear | | | |
| Sterile Water for | N/A | N/A | 1456.1 |
| Irrigation, USP | | | |
| Total | 100.0 | 51.0 | 3094.3 |

Formulation of capsules comprising sumatriptan and promethazine was performed as described Table 8 and detailed below.

TABLE 8
Formulation and Encapsulation - 100 capsules

| | Manufacturer | Percent | | Batch |
|---|---|---|---|---|
| Ingredient | Lot Number | w/w | mg/capsule | Quantity (g) |
| Sumatriptan | Xcelience | 80.6% | 212.06 | 556.7 |
| particulates | IP00048 | | | |
| Promethazine | Xcelience | 19.4% | 51.0 | 133.9 |
| particulates | IP00047 | | | |
| Total | | | 263.06 | |
| Capsules, | Capsugel | N/A | 96.0 | 264.9 |
| Size 0 Coni- | RM00895 | | (1 capsule) | (2500 capsules) |
| Snap white | | | | |
| opaque gelatin | | | | |
| capsules | | | | |

| | | |
|---|---|---|
| Total | 359.06 | 955.5 |

Table 8. The batch weights for sumatriptan and promethazine particulates represented an approximate 5% overage in order to yield 2,500 acceptable capsules based upon the theoretical batch size of 2,625 capsules. The batch weight for 264.9 g of capsules represented an approximate 5% overage in order to cover potential losses during manufacture.

212.06 mg of sumatriptan 90 mg coated particulates (with a +/−5% range of 201.5 to 222.6 mg) were placed in Size 0 capsules. Next, 51.0 mg of Promethazine HCL 25 mg coated particulates (with a +/−5% range of 48.5 to 53.5 mg) were placed in the Size 0 capsules. The capsules were packaged in opaque HDPE bottles.

### Example 3. Dissolution Measurements by USP Basket Method

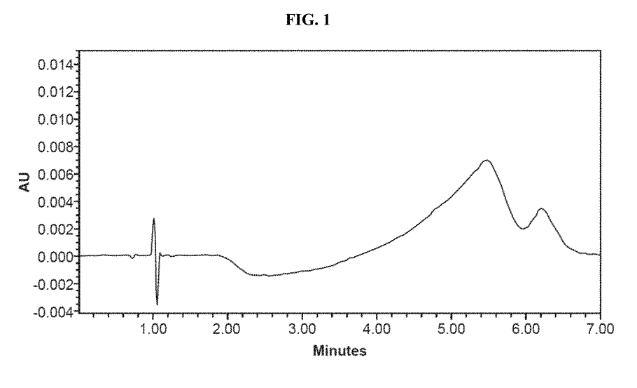

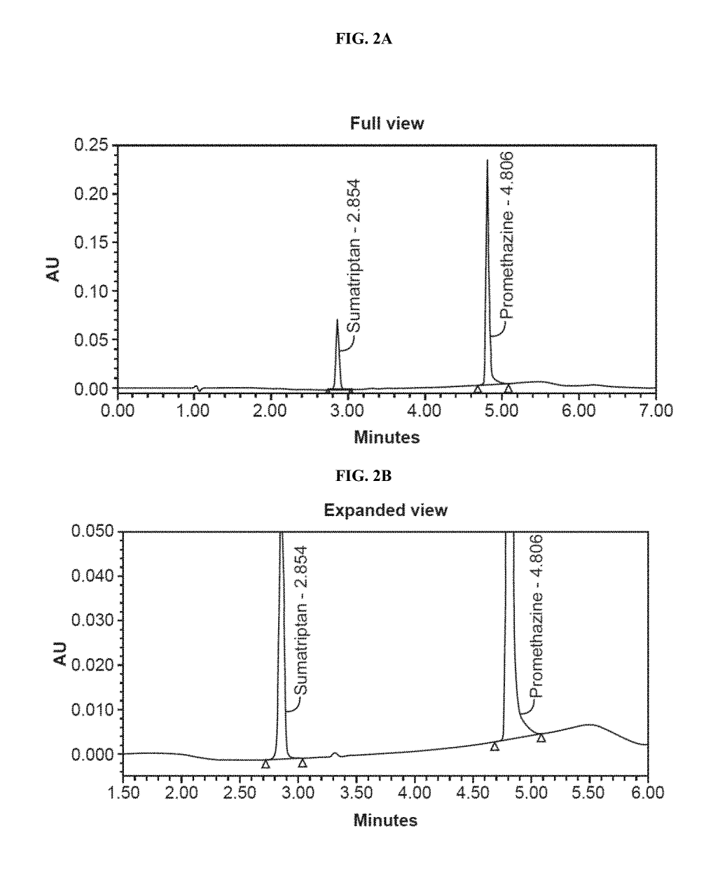

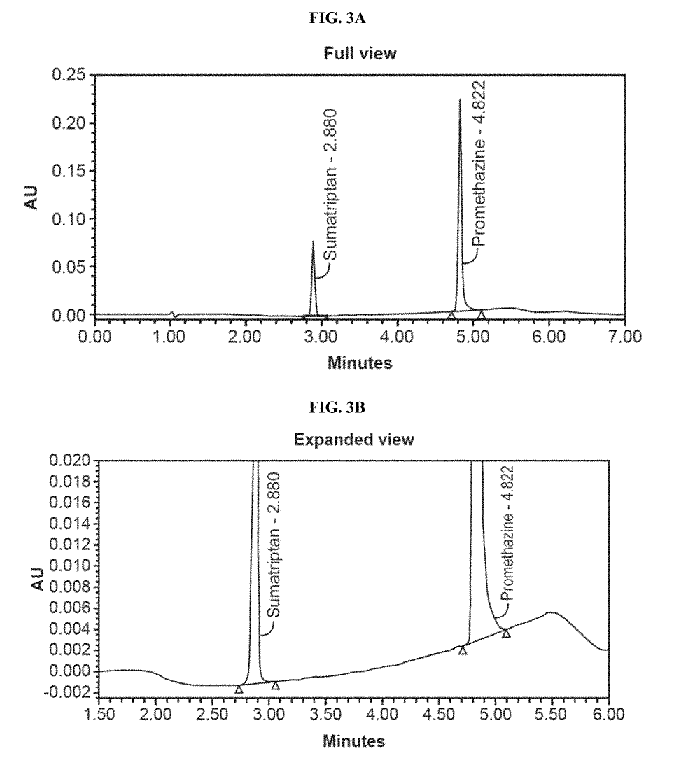

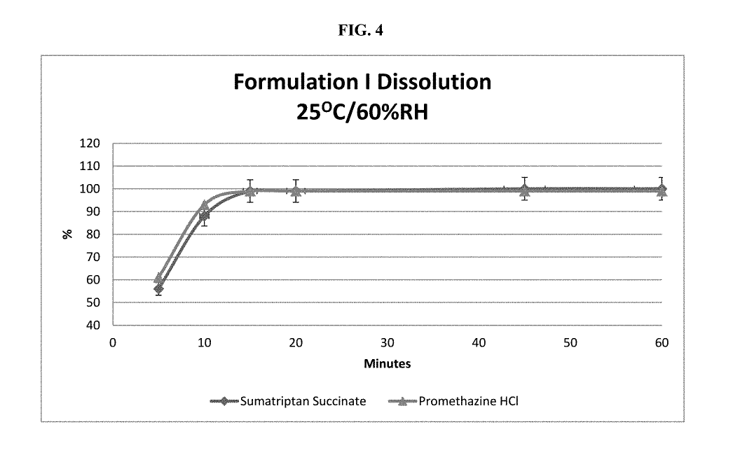

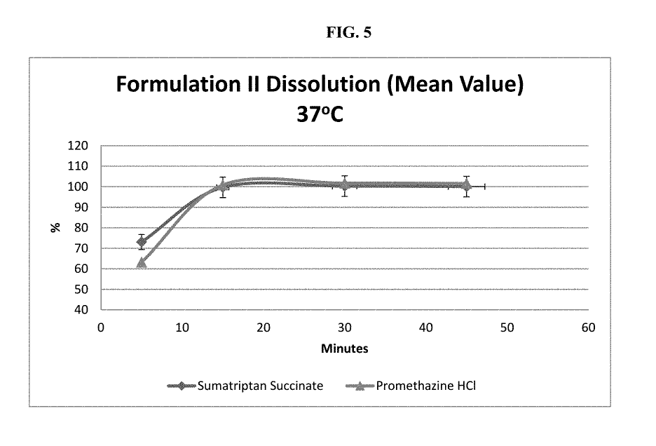

Dissolution studies were conducted to measure the rates of dissolution of active ingredients. Dissolution tests were run using a USP Apparatus 1 (Basket Apparatus) with a dissolution fluid of 900 mL de-aerated 0.01 N HCl (i.e., pH 2.0) at 37.0+/−0.5° C. Dissolution samples were analyzed by HPLC. Chromatographs for the dissolution medium, standard samples, and test sample as shown in FIGS. 1, 2A-2B, and 3A-3B. The dissolution results for Formulation I and Formulation II are shown in FIG. 4 and FIG. 5.

Dissolution medium of 0.01N HCl was prepared by mixing well approximately 5 mL of concentrated (12N) Hydrochloric Acid with 6 L of water. Stock promethazine HCl standard solution was prepared by adding approximately 30 mL of dissolution medium to 14.0 mg of dried Promethazine Hydrochloride USP reference standard in a 50 mL volumetric flash, diluted to volume with dissolution media, and mixed well. Working Standard Solution was prepared by first mixing well 14.0 mg of Sumatriptan Succinate USP reference standard with approximately 60 mL of dissolution medium and then pipetting 10.0 mL of Promethazine Hydrochloride stock solution into the prepared Sumatriptan Succinate solution. The resulting solution was diluted to volume with dissolution medium and mixed well. Nominal concentration for Sumatriptan was 0.10 mg/mL (as a free base) and Promethazine HCl was 0.028 mg/mL in the Sumatriptan Succinate and Promethazine HCl Working Standard A and B. The label claim for Sumatriptan was as a free base and therefore the final standard concentration was converted accordingly multiplying by the salt-to-base conversion factor: (295.40/413.49).

The dissolution apparatus used was USP Apparatus I (Basket) with a speed of 100 rpm at 37.0° C.±0.5° C. Dissolution medium (900 mL) was Helium sparged for at least 10 minutes. N=6 samples were tested, one per sinker and per vessel. At each time point of 5, 15, 30, and 45 minutes, a 5 mL aliquot from each dissolution vessel was filtered through a 0.45 μm Nylon membrane syringe filter before HPLC analysis.

HPLC conditions: Flow rate: 1.0 mL/min; Injection Volume: 5 μL; Column Temperature: 40° C.; Wavelengths: 254 nm; Run Time: 7 minutes; Mobile Phase A was 0.2% TFA in Water, which was prepared by mixing well 2.0 mL of trifluoroacetic acid with 1 L of water. Mobile Phase B: 0.2% TFA in Acetonitrile, which was prepared by mixing well 2.0 mL of trifluoroacetic acid to 1 L of acetonitrile; and Gradient used was as follows in Table 9.

TABLE 9

| Time | % A | % B |
|---|---|---|
| (minutes) | (Buffer) | (ACN) |
| Initial | 90 | 10 |
| 4.0 | 40 | 60 |
| 4.1 | 90 | 10 |
| 7.0 | 90 | 10 |

Approximate Retention Time for sumatriptan and promethazine was 2.8 minutes and 4.8 minutes respectively.

Calculation.

Calculations for percent release were conducted using the following formulas. Percent Release of Promethazine (Profile):

$$\% Released = [ ( \frac{{R}_{u}}{{R}_{s}} \times {C}_{std} \times {V}_{d} ) + {\sum }_{i = 1}^{n - 1} ( \frac{{R}_{i}}{{R}_{s}} \times {C}_{std} \times {V}_{i} ) ] \times ( \frac{1}{L C} ) \times 100$$

Where:
Rᵤ=Peak area of Promethazine in the sample preparation
Rₛ=Mean peak area of Promethazine in all Working Standard A injections
C_{std}=Working Standard A concentration of Promethazine Hydrochloride, adjusted for purity (μg/mL)
V_{d}=Volume of dissolution medium at the pull time (mL)
Rᵢ=Peak area of Promethazine obtained from the sample preparation at the individual pull points
Vᵢ=Volume of the sample removed from the vessel at the pull point (mL)
LC=Label claim (25 mg or 25000 μg)
100=Conversion to percent

Percent Release of Sumatriptan (Profile):

$$\% Released = [ ( \frac{{R}_{u}}{{R}_{s}} \times {C}_{std} \times {V}_{d} ) + {\sum }_{i = 1}^{n - 1} ( \frac{{R}_{i}}{{R}_{s}} \times {C}_{std} \times {V}_{i} ) ] \times ( \frac{1}{L C} ) \times 100$$

Where:
Rᵤ=Peak area of Sumatriptan in the sample preparation
Rₛ=Mean peak area of Sumatriptan in all Working Standard A injections
C_{std}=Working Standard A concentration of Sumatriptan, succinate adjusted for purity and conversion to free base (μg/mL)
V_{d}=Volume of dissolution medium at the pull time (mL)
Rᵢ=Peak area of Sumatriptan obtained from the sample preparation at the individual pull points
Vᵢ=Volume of the sample removed from the vessel at the pull point (mL)
LC=Label claim (90 mg or 90000 μg)
100=Conversion to percent

Dissolution measurements for Formulation I measured by USP Apparatus 1 (Basket) rotating at 100 rpm are shown in Table 10. See also FIG. 4.

TABLE 10

| | Minutes | | | | | |
|---|---|---|---|---|---|---|
| | 5 | 10 | 15 | 20 | 45 | 60 |
| Sumatriptan Succinate % Dissolution | 56 | 88 | 99 | 99 | 100 | 100 |
| Promethazine HCl % Dissolution | 61 | 93 | 99 | 99 | 99 | 99 |

Dissolution measurements for Formulation II measured by USP Apparatus 1 (Basket) rotating at 100 rpm are shown in Table 11 and Table 12. See also FIG. 5.

TABLE 11
Dissolved Percent for Component: Promethazine
Channel: A1100 DAD AU Ch1

| | | | Injec- | 5.0 | 15.0 | 30.0 | 45.0 |
|---|---|---|---|---|---|---|---|
| | Bath | Vessel | tion | min | min | min | min |
| 1 | A | 1 | 1 | 64.14 | 101.57 | 102.40 | 102.26 |
| 2 | A | 2 | 1 | 68.86 | 103.65 | 104.21 | 104.06 |
| 3 | A | 3 | 1 | 51.79 | 100.02 | 101.14 | 101.14 |
| 4 | A | 4 | 1 | 57.94 | 100.55 | 101.85 | 101.66 |
| 5 | A | 5 | 1 | 72.94 | 98.05 | 98.39 | 98.27 |
| 6 | A | 6 | 1 | 63.54 | 101.20 | 102.40 | 102.15 |
| Mean | A | | | 63.20 | 100.84 | 101.73 | 101.59 |
| % RSD | | | | 11.961 | 1.831 | 1.893 | 1.873 |

TABLE 12
Dissolved Percent for Component: Sumatriptan
Channel: A1100 DAD AU Ch1

| | | | Injec- | 5.0 | 15.0 | 30.0 | 45.0 |
|---|---|---|---|---|---|---|---|
| | Bath | Vessel | tion | min | min | min | min |
| 1 | A | 1 | 1 | 75.96 | 98.80 | 99.05 | 98.84 |
| 2 | A | 2 | 1 | 76.01 | 98.52 | 98.74 | 98.62 |
| 3 | A | 3 | 1 | 62.76 | 99.76 | 100.89 | 100.99 |
| 4 | A | 4 | 1 | 70.64 | 102.00 | 102.54 | 102.11 |
| 5 | A | 5 | 1 | 82.71 | 98.89 | 99.17 | 98.97 |
| 6 | A | 6 | 1 | 70.25 | 100.15 | 101.36 | 100.97 |
| Mean | A | | | 73.06 | 99.69 | 100.29 | 100.08 |
| % RSD | | | | 9.284 | 1.294 | 1.531 | 1.460 |

### Example 4. Capsules

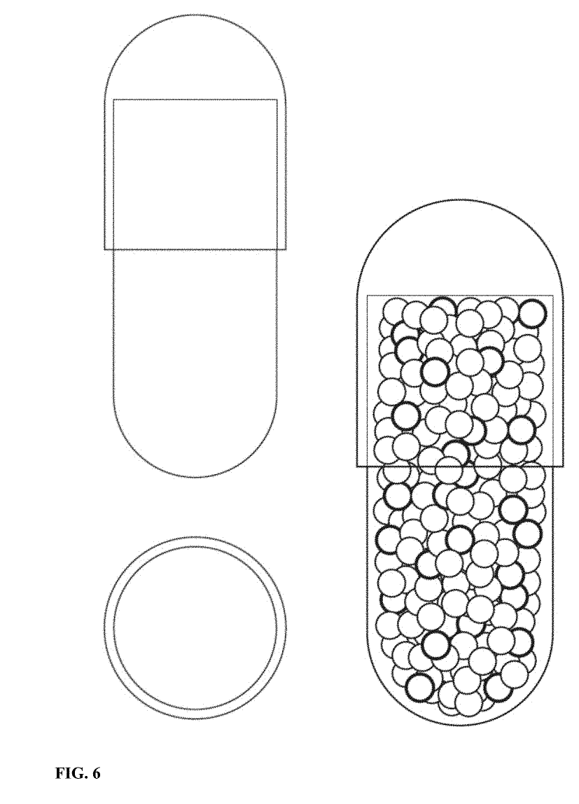

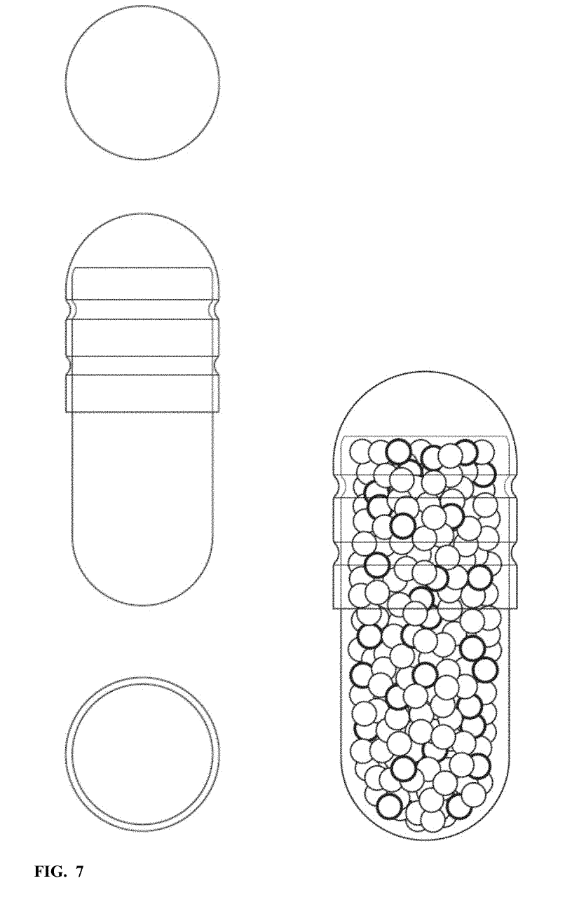

Suitable capsule designs for housing pharmaceutical compositions disclosed herein are shown in FIGS. 6 and 7. For the capsule depicted in FIG. 7, each capsule weighs about 96±6 mg. Capsule features are detailed in Table 13.

TABLE 13
Approximate capacity of each capsule

| | Capsule volume: |
|---|---|
| | 0.68 ml |
| Powder density: | Amount in capsule: |
| 0.6 g/ml | 408 mg |
| 0.8 g/ml | 544 mg |
| 1.0 g/ml | 680 mg |
| 1.2 g/ml | 816 mg |

In the case of the capsule in FIG. 6, approximate length of the capsule parts was: body: 0.726±0.018 inches or 18.44±0.46 mm; and cap: 0.422±0.018 inches or 10.72±0.46 mm. Approximate external diameter was body: 0.289±0.002 inches or 7.34±0.06 mm; and cap 0.300±0.002 inches or 7.61±0.06 mm. Approximate overall closed length was 0.854±0.012 inches or 21.7±0.3 mm.

### Example 5. Stability Study

Formulation I and Formulation II were examined for their stability over time (T), initial reading and one month, under two different environmental conditions: 40° C. and 75% resting humidity (RH) or 25° C. and 60% RH. The samples were then analyzed under the following HPLC Conditions: HPLC System (Agilent or Waters) equipped with DAD or PDA with Phenomenex Luna C18(2), 5 μm, 4.6×250 mm Column; Mobile Phase A: 24 mM Sodium Phosphate Buffer Solution, pH 4.0—(1 L); Mobile Phase B: 100% Acetonitrile—(1 L); Flow rate: 0.8 mL/min: Injection Volume: 5 μL; Column Temperature: 45° C.; Sample Temperature: 5° C.; Wavelength: 228 nm (for Sumatriptan and its related substances); 254 nm (for Promethazine and its related substances); Run Time: 50 minutes; Needle Wash: 50/50 Water/Acetonitrile (1 cycle). Elution conditions are summarized in Table 14.

TABLE 14
Elution Gradient:

| Time | | |
|---|---|---|
| (minutes) | % A | % B |
| Initial | 95 | 5 |
| 20 | 60 | 40 |
| 28 | 10 | 90 |
| 42 | 10 | 90 |
| 43 | 95 | 5 |
| 50 | 95 | 5 |

Calculations

Assay—Percent Label Claim:

$$\% L C = \frac{{A}_{sample}}{{A}_{STD}} \times {C}_{STD} \times \frac{D}{L C \times {N}_{C}} \times 100$$

Where:
Aₛₐₘₚₗₑ=Peak area of Promethazine or Sumatriptan in sample preparation
A_{STD}=Average peak area of Promethazine or Sumatriptan in all Standard A injections
C_{STD}=Concentration of Promethazine hydrochloride and Sumatriptan Standard A (μg/mL), including purity and conversion to free base (Sumatriptan only)
N_{C}=Number of capsules used
LC=Label Claim: 90 mg (Sumatriptan) or 25 mg (Promethazine Hydrochloride)
D=Dilution Factor
100=Conversion to percentage

% Area for Related Substances:

$$\% Area = \frac{{A}_{RI}}{{A}_{Main} + {A}_{Sum RS}} \times 100 =$$

Where:
A_{RS}: Peak area of Related Substance in the sample preparation
A_{Main}: Peak area of Promethazine or Sumatriptan in sample preparation
A_{Sum RI}: Sum of all related Substances area ≧LOQ in sample preparation*
100: Conversion to percentage *Peaks between 0-17 minutes were considered Sumatriptan-related. Peaks from 17-40 minutes were Promethazine-related.

Assay Results

Results from stability studies of Formulation I and Formulation II are show in Table 15, below.

TABLE 15
Concentrations of sumatriptan and promethazine hydrochloride
measured in the HPLC assay relative to their respective standards

| | | Time Point | | |
|---|---|---|---|---|
| | | Initial | T = 1 M | T = 1 M |
| | | Condition | | |
| | | | | 25° C./60% |
| | | T = 0 | 40° C./75% RH | RH |
| Formulation I | Sumatriptan | 102.4 | 92.0 | 90.4 |
| | Promethazine | 98.7 | 93.5 | 90.0 |
| | hydrochloride | | | |
| Formulation II | Sumatriptan | 101.1 | 95.4 | 100.4 |
| | Promethazine | 102.6 | 100.2 | 101.3 |
| | hydrochloride | | | |

### Example 6. Clinical Study for Formulation II

A clinical study will be conducted in order to assess the pharmacokinetics of Formulation II. In order to obtain controlled results, the study will compare data from subjects treated with Formulation II to data obtained from subjects treated with comparator products. Over the course of treatment, observations aside from pharmacokinetic analysis are to be considered. Categories for additional findings to be considered include, without limitation, safety, patient pre-disposition correlations (genetic or otherwise), and efficacy findings. The study will be for a single-dose, open-label, randomized, three-period, three-treatment crossover study in which healthy adult subjects receive a single dose of Formulation II (90 mg sumatriptan succinate/25 mg promethazine HCl capsule) in one period, a separate single dose of IMITREX (sumatriptan succinate) tablet 100 mg in one period, and a separate single dose of promethazine HCl tablet 25 mg in one period, under fasted conditions. More specifically, subjects will receive each of the treatments listed below in randomized fashion during the three treatment periods:

### Treatment A: Test Formulation

Formulation II (sumatriptan succinate/promethazine HCl)
90 mg/25 mg capsule
Dose=1×90 mg/25 mg capsule

### Treatment B: Comparator Product

IMITREX (sumatriptan succinate) tablet, 100 mg
Dose=1×100 mg tablet
GlaxoSmithKline

### Treatment C: Comparator Product

Promethazine HCl tablet, 25 mg
Dose=1×25 mg tablet
Zydus Pharmaceuticals

Each drug administration will be separated by a washout period of at least 7 days. Each dose will be orally administered along with approximately 240 mL (8 fl. oz.) of room temperature water following a 10-hour overnight fast. After dosing, no food will be allowed until 4 hours postdose. Except for the 240 mL of room temperature water provided with the dose, no water consumption will be allowed for 1 hour prior through 1 hour after dose. Meals will be the same and scheduled at approximately the same times relative to dose for each study period.

During each study period, 4 mL blood samples will be obtained prior to each dosing and following each dose at selected times through 48 hours postdose. Plasma pharmacokinetic samples will be analyzed for sumatriptan and promethazine using validated analytical methods. Appropriate pharmacokinetic parameters will be calculated for each formulation using non-compartmental methods. In addition, blood and urine will be collected for clinical laboratory testing at screening and at the end of the study.

Each subject dosed in this study will receive an assigned treatment sequence based on a randomization schedule prepared by the clinical site. Subjects will be randomized to receive either Treatment A, Treatment B, or Treatment C during the first study period. After a minimum washout of 7 days, each subject will cross over to receive an alternate treatment. After another minimum washout of 7 days, subjects will cross over to receive the final treatment. At the completion of the study, each subject will have received a single dose of Treatment A, a single dose of Treatment B, and a single dose of Treatment C.

Plasma samples will be analyzed for sumatriptan and promethazine using validated assays. The samples from all evaluable subjects completing at least one study period will be analyzed. Pharmacokinetic parameters for sumatriptan and promethazine will be calculated using non-compartmental analysis with 10% adjustment for the 10 mg difference in the doses of sumatriptan. The following pharmacokinetic parameters will be determined.

The maximum plasma concentration (Cₘₐₓ) and time to Cₘₐₓ (Tₘₐₓ) will be taken directly from the data. The elimination rate constant, λz, will be calculated as the negative of the slope of the terminal log-linear segment of the plasma concentration-time curve; the range of data to be used will be determined by visual inspection of a semi-logarithmic plot of concentration vs. time. Elimination half-life (T½) will be calculated according to the following equation: T½=0.693/λ_{Z}.

Area under the curve to the final sample with a concentration greater than the limit of quantitation (LOQ), (AUCₗₐₛₜ), will be calculated using the linear trapezoidal method and extrapolated to infinity using: AUC_{inf}=AUCₗₐₛₜ+Clast/λ_{Z} where Cₗₐₛₜ is the final concentration ≧LOQ. In addition, the following partial AUCs will be calculated for promethazine and sumatriptan: AUC_{(0-0.25)}, AUC_{(0-0.5)}, AUC_{(0-0.75)}, AUC_{(0-1.0)}, AUC_{(0-1.5)}, AUC_{(0-2.0)}, AUC_{(0-3.0)}, and AUC_{(0-4.0)}.

Comparison of the log-transformed pharmacokinetic parameters Cₘₐₓ, AUCₗₐₛₜ, and AUC_{inf} for sumatriptan and promethazine across treatments will be performed using an analysis of variance (ANOVA) model and the two one-sided t-tests procedure. Partial AUCs [AUC_{(0-0.25)}, AUC_{(0-0.5)}, AUC_{(0-0.75)}, AUC_{(0-1.0)}, AUC_{(0-1.5)}, AUC_{(0-2.0)}, AUC_{(0-3.0)}, and AUC_{(0-4.0)}] for sumatriptan and promethazine will be included in the analysis for comparisons of early systemic exposure across treatments. The ANOVA model will include factors for sequence, subject within sequence, treatment, and period. The ratios of the geometric means (test to reference) and 90% confidence intervals will be reported. Statistical analyses will be performed using appropriate software, e.g. PHOENIX WINNONLIN (Version 6.3, Pharsight Corporation) and/or SAS (Version 9.3, SAS Institute Inc.).

### Example 7. Dissolution Measurement by USP Paddle Method

A dissolution study is to be conducted to measure the rates of dissolution of active ingredients. This study will use a USP Rotating Paddle Apparatus 2 with an automated sampling station (e.g., VK-8000 or equivalent). A dissolution fluid of 900 mL of de-aerated 0.01 N HCl (i.e., pH 2.0), maintained at 37.0+/−0.5° C., will be used during the dissolution procedure. The fluid will be prepared by diluting 5 mL of concentrated HCl in 6000 mL of de-aerated water, and mixed. To measure peaks, a dual wavelength detector (e.g., Hitachi L-2420) will be used, or alternatively, two separate chromatographic systems will be used in order to measure the peaks at two different wavelengths.

In order to prepare standard solutions, each ingredient will be weighed into a 50 mL volumetric flask, and diluted to volume with dissolution media. The resulting solution will be mixed to form a stock solution. Different ingredients will be similarly prepared to provide stock solutions (e.g., promethazine HCl, triptan). 2 mL each of stock standard solutions will be diluted with dissolution fluid and mixed to produce a final standard solution.

Dissolution test solutions will be prepared in 900 mL of 0.01 N HCl (i.e., pH 2.0) using the USP Rotating Paddle Apparatus at 50 μM. An aliquot of the dissolution solution will be filtered and a 50-pL aliquot is chromatographed on a 50-mm×4.6-mm (i.d.) Waters sunFire™ C18, 3.5-μm particle size column using a gradient HPLC method. Mobile phase A will consist of water/acetonitrile/TFA, 950/50/2 (v/v/v) and mobile phase B will consist of water/acetonitrile/TFA, 50/950/1.5 (v/v/v). The flow rate will be 2.0 mL/minute.

The amount of triptan released will be determined at 300 nm by comparing the area obtained for the peak due to triptan in the chromatogram of the dissolution test solution to that obtained for the corresponding peak in a chromatogram of a standard solution. The amount of promethazine HCl released will be determined at 230 nm by comparing the area obtained for the peak due to promethazine HCl in the chromatogram of the dissolution test solution to that obtained for the corresponding peak in a chromatogram of a standard solution.

Paddle speed will be 50 rpm and pull volume will be 10 mL. Pull points of 5, 10, 15, 20, 25, 30, 45 and 60 minutes will be used. The amount of each component dissolved in the dissolution medium will be determined by HPLC. This protocol will use a high purity, bonded C18 stationary phase and a binary mobile phase consisting of an appropriate buffer and organic modifier.

To begin the dissolution procedure, 900 mL of dissolution fluid will be preheated to 37° C. and placed into each vessel. A pharmaceutically active agent as described herein will be weighed and placed in vessels respectively. At prescribed time intervals, 5 mL aliquot of the dissolution fluid will be drawn using the automated sampling station equipped with a 35 μm full flow filter connected to a sampling probe. Filtrate will be allowed to cool to room temperature, to produce a final sample solution. Fluid withdrawn will not be replaced. Samples will be injected in HPLC for analysis after a baseline is established. Peak area responses will be measured for the pharmaceutically active agent. The resolution between each peak will be calculated, as well as the tailing factor. 50 μL aliquots of standard and sample solutions will be subjected to liquid chromatography.

The amount of a pharmaceutically active agent in a particulate or capsule will be determined by comparing the area obtained for the peak due to the agent in a chromatogram of the dissolution test solution to that obtained for the corresponding peak in a chromatogram of a standard solution.

### Example 8. Pharmaceutical Compositions

Pharmaceutical compositions will be designed comprising a combination of one or more triptan molecules and one or more antiemetics. Pharmaceutical compositions formed include the combinations of active ingredients listed Table 16, or pharmaceutically acceptable salts thereof. Pharmaceutical compositions, such as those listed in Table 16, will be studied for effectiveness in the treatment of pain.

TABLE 16
Drug Pharmaceutical Compositions

| Composition No. | Triptan | Antiemetic |
|---|---|---|
| 1 | Sumatriptan | Promethazine |
| 2 | Sumatriptan | Aprepitant |
| 3 | Sumatriptan | Dronabinol |
| 4 | Sumatriptan | Perphenazine |
| 5 | Sumatriptan | Palonosetron |
| 6 | Sumatriptan | Trimethyobenzamide |
| 7 | Sumatriptan | Metoclopromide |
| 8 | Sumatriptan | Domperidone |
| 9 | Sumatriptan | Prochlorperazine |
| 10 | Sumatriptan | Chlorpromazine |
| 11 | Sumatriptan | Trimethobenzamide |
| 12 | Sumatriptan | Ondansetron |
| 13 | Sumatriptan | Granisetron |
| 14 | Sumatriptan | Hydroxyzine |
| 15 | Sumatriptan | Acetylleucine Monoethanolamine |
| 16 | Sumatriptan | Alizapride |
| 17 | Sumatriptan | Azasetron |
| 18 | Sumatriptan | Benzquinamide |
| 19 | Sumatriptan | Bietanautine |
| 20 | Sumatriptan | Bromopride |
| 21 | Sumatriptan | Buclizine |
| 22 | Sumatriptan | Clebopride |
| 23 | Sumatriptan | Cyclizine |
| 24 | Sumatriptan | Dimenhydrinate |
| 25 | Sumatriptan | Diphenidol |
| 26 | Sumatriptan | Dolasetron |
| 27 | Sumatriptan | Meclizine |
| 28 | Sumatriptan | Methallatal |
| 29 | Sumatriptan | Metopimazine |
| 30 | Sumatriptan | Nabilone |
| 31 | Sumatriptan | Oxyperndyl |
| 32 | Sumatriptan | Pipamazine |
| 33 | Sumatriptan | Scopolamine |
| 34 | Sumatriptan | Sulpiride |
| 35 | Sumatriptan | Tetrahydrocannabinol |
| 36 | Sumatriptan | Thiethylperazine |
| 37 | Sumatriptan | Thioproperazine |
| 38 | Sumatriptan | Tropisetron |
| 39 | Sumatriptan | Droperidol |
| 40 | Sumatriptan | Haloperidol |
| 41 | Sumatriptan | Prochloperazine |
| 42 | Sumatriptan | Metoclopramide |
| 43 | Sumatriptan | Diphenhydramine |
| 44 | Sumatriptan | Cannabis |
| 45 | Sumatriptan | Midazolam |
| 46 | Sumatriptan | Lorazepam |
| 47 | Sumatriptan | Hyoscine |
| 48 | Sumatriptan | Dexamethasone |
| 49 | Sumatriptan | Emetrol |
| 50 | Sumatriptan | Propofol |
| 51 | Almotriptan | Promethazine |
| 52 | Almotriptan | Aprepitant |
| 53 | Almotriptan | Dronabinol |
| 54 | Almotriptan | Perphenazine |
| 55 | Almotriptan | Palonosetron |
| 56 | Almotriptan | Trimethyobenzamide |
| 57 | Almotriptan | Metoclopromide |
| 58 | Almotriptan | Domperidone |
| 59 | Almotriptan | Prochlorperazine |
| 60 | Almotriptan | Chlorpromazine |
| 61 | Almotriptan | Trimethobenzamide |
| 62 | Almotriptan | Ondansetron |
| 63 | Almotriptan | Granisetron |
| 64 | Almotriptan | Hydroxyzine |
| 65 | Almotriptan | Acetylleucine Monoethanolamine |
| 66 | Almotriptan | Alizapride |
| 67 | Almotriptan | Azasetron |
| 68 | Almotriptan | Benzquinamide |
| 69 | Almotriptan | Bietanautine |
| 70 | Almotriptan | Bromopride |
| 71 | Almotriptan | Buclizine |
| 72 | Almotriptan | Clebopride |
| 73 | Almotriptan | Cyclizine |
| 74 | Almotriptan | Dimenhydrinate |
| 75 | Almotriptan | Diphenidol |
| 76 | Almotriptan | Dolasetron |
| 77 | Almotriptan | Meclizine |
| 78 | Almotriptan | Methallatal |
| 79 | Almotriptan | Metopimazine |
| 80 | Almotriptan | Nabilone |
| 81 | Almotriptan | Oxyperndyl |
| 82 | Almotriptan | Pipamazine |
| 83 | Almotriptan | Scopolamine |
| 84 | Almotriptan | Sulpiride |
| 85 | Almotriptan | Tetrahydrocannabinol |
| 86 | Almotriptan | Thiethylperazine |
| 87 | Almotriptan | Thioproperazine |
| 88 | Almotriptan | Tropisetron |
| 89 | Almotriptan | Droperidol |
| 90 | Almotriptan | Haloperidol |
| 91 | Almotriptan | Prochloperazine |
| 92 | Almotriptan | Metoclopramide |
| 93 | Almotriptan | Diphenhydramine |
| 94 | Almotriptan | Cannabis |
| 95 | Almotriptan | Midazolam |
| 96 | Almotriptan | Lorazepam |
| 97 | Almotriptan | Hyoscine |
| 98 | Almotriptan | Dexamethasone |
| 99 | Almotriptan | Emetrol |
| 100 | Almotriptan | Propofol |
| 101 | Forvatriptan | Promethazine |
| 102 | Forvatriptan | Aprepitant |
| 103 | Forvatriptan | Dronabinol |
| 104 | Forvatriptan | Perphenazine |
| 105 | Forvatriptan | Palonosetron |
| 106 | Forvatriptan | Trimethyobenzamide |
| 107 | Forvatriptan | Metoclopromide |
| 108 | Forvatriptan | Domperidone |
| 109 | Forvatriptan | Prochlorperazine |
| 110 | Forvatriptan | Chlorpromazine |
| 111 | Forvatriptan | Trimethobenzamide |
| 112 | Forvatriptan | Ondansetron |
| 113 | Forvatriptan | Granisetron |
| 114 | Forvatriptan | Hydroxyzine |
| 115 | Forvatriptan | Acetylleucine Monoethanolamine |
| 116 | Forvatriptan | Alizapride |
| 117 | Forvatriptan | Azasetron |
| 118 | Forvatriptan | Benzquinamide |
| 119 | Forvatriptan | Bietanautine |
| 120 | Forvatriptan | Bromopride |
| 121 | Forvatriptan | Buclizine |
| 122 | Forvatriptan | Clebopride |
| 123 | Forvatriptan | Cyclizine |
| 124 | Forvatriptan | Dimenhydrinate |
| 125 | Forvatriptan | Diphenidol |
| 126 | Forvatriptan | Dolasetron |
| 127 | Forvatriptan | Meclizine |
| 128 | Forvatriptan | Methallatal |
| 129 | Forvatriptan | Metopimazine |
| 130 | Forvatriptan | Nabilone |
| 131 | Forvatriptan | Oxyperndyl |
| 132 | Forvatriptan | Pipamazine |
| 133 | Forvatriptan | Scopolamine |
| 134 | Forvatriptan | Sulpiride |
| 135 | Forvatriptan | Tetrahydrocannabinol |
| 136 | Forvatriptan | Thiethylperazine |
| 137 | Forvatriptan | Thioproperazine |
| 138 | Forvatriptan | Tropisetron |
| 139 | Forvatriptan | Droperidol |
| 140 | Forvatriptan | Haloperidol |
| 141 | Forvatriptan | Prochloperazine |
| 142 | Forvatriptan | Metoclopramide |
| 143 | Forvatriptan | Diphenhydramine |
| 144 | Forvatriptan | Cannabis |
| 145 | Forvatriptan | Midazolam |
| 146 | Forvatriptan | Lorazepam |
| 147 | Forvatriptan | Hyoscine |
| 148 | Forvatriptan | Dexamethasone |
| 149 | Forvatriptan | Emetrol |
| 150 | Forvatriptan | Propofol |
| 151 | Rizatriptan | Promethazine |
| 152 | Rizatriptan | Aprepitant |
| 153 | Rizatriptan | Dronabinol |
| 154 | Rizatriptan | Perphenazine |
| 155 | Rizatriptan | Palonosetron |
| 156 | Rizatriptan | Trimethyobenzamide |
| 157 | Rizatriptan | Metoclopromide |
| 158 | Rizatriptan | Domperidone |
| 159 | Rizatriptan | Prochlorperazine |
| 160 | Rizatriptan | Chlorpromazine |
| 161 | Rizatriptan | Trimethobenzamide |
| 162 | Rizatriptan | Ondansetron |
| 163 | Rizatriptan | Granisetron |
| 164 | Rizatriptan | Hydroxyzine |
| 165 | Rizatriptan | Acetylleucine Monoethanolamine |
| 166 | Rizatriptan | Alizapride |
| 167 | Rizatriptan | Azasetron |
| 168 | Rizatriptan | Benzquinamide |
| 169 | Rizatriptan | Bietanautine |
| 170 | Rizatriptan | Bromopride |
| 171 | Rizatriptan | Buclizine |
| 172 | Rizatriptan | Clebopride |
| 173 | Rizatriptan | Cyclizine |
| 174 | Rizatriptan | Dimenhydrinate |
| 175 | Rizatriptan | Diphenidol |
| 176 | Rizatriptan | Dolasetron |
| 177 | Rizatriptan | Meclizine |
| 178 | Rizatriptan | Methallatal |
| 179 | Rizatriptan | Metopimazine |
| 180 | Rizatriptan | Nabilone |
| 181 | Rizatriptan | Oxyperndyl |
| 182 | Rizatriptan | Pipamazine |
| 183 | Rizatriptan | Scopolamine |
| 184 | Rizatriptan | Sulpiride |
| 185 | Rizatriptan | Tetrahydrocannabinol |
| 186 | Rizatriptan | Thiethylperazine |
| 187 | Rizatriptan | Thioproperazine |
| 188 | Rizatriptan | Tropisetron |
| 189 | Rizatriptan | Droperidol |
| 190 | Rizatriptan | Haloperidol |
| 191 | Rizatriptan | Prochloperazine |
| 192 | Rizatriptan | Metoclopramide |
| 193 | Rizatriptan | Diphenhydramine |
| 194 | Rizatriptan | Cannabis |
| 195 | Rizatriptan | Midazolam |
| 196 | Rizatriptan | Lorazepam |
| 197 | Rizatriptan | Hyoscine |
| 198 | Rizatriptan | Dexamethasone |
| 199 | Rizatriptan | Emetrol |
| 200 | Rizatriptan | Propofol |
| 201 | Zolmitriptan | Promethazine |
| 202 | Zolmitriptan | Aprepitant |
| 203 | Zolmitriptan | Dronabinol |
| 204 | Zolmitriptan | Perphenazine |
| 205 | Zolmitriptan | Palonosetron |
| 206 | Zolmitriptan | Trimethyobenzamide |
| 207 | Zolmitriptan | Metoclopromide |
| 208 | Zolmitriptan | Domperidone |
| 209 | Zolmitriptan | Prochlorperazine |
| 210 | Zolmitriptan | Chlorpromazine |
| 211 | Zolmitriptan | Trimethobenzamide |
| 212 | Zolmitriptan | Ondansetron |
| 213 | Zolmitriptan | Granisetron |
| 214 | Zolmitriptan | Hydroxyzine |
| 215 | Zolmitriptan | Acetylleucine Monoethanolamine |
| 216 | Zolmitriptan | Alizapride |
| 217 | Zolmitriptan | Azasetron |
| 218 | Zolmitriptan | Benzquinamide |
| 219 | Zolmitriptan | Bietanautine |
| 220 | Zolmitriptan | Bromopride |
| 221 | Zolmitriptan | Buclizine |
| 222 | Zolmitriptan | Clebopride |
| 223 | Zolmitriptan | Cyclizine |
| 224 | Zolmitriptan | Dimenhydrinate |
| 225 | Zolmitriptan | Diphenidol |
| 226 | Zolmitriptan | Dolasetron |
| 227 | Zolmitriptan | Meclizine |
| 228 | Zolmitriptan | Methallatal |
| 229 | Zolmitriptan | Metopimazine |
| 230 | Zolmitriptan | Nabilone |
| 231 | Zolmitriptan | Oxyperndyl |
| 232 | Zolmitriptan | Pipamazine |
| 233 | Zolmitriptan | Scopolamine |
| 234 | Zolmitriptan | Sulpiride |
| 235 | Zolmitriptan | Tetrahydrocannabinol |
| 236 | Zolmitriptan | Thiethylperazine |
| 237 | Zolmitriptan | Thioproperazine |
| 238 | Zolmitriptan | Tropisetron |
| 239 | Zolmitriptan | Droperidol |
| 240 | Zolmitriptan | Haloperidol |
| 241 | Zolmitriptan | Prochloperazine |
| 242 | Zolmitriptan | Metoclopramide |
| 243 | Zolmitriptan | Diphenhydramine |
| 244 | Zolmitriptan | Cannabis |
| 245 | Zolmitriptan | Midazolam |
| 246 | Zolmitriptan | Lorazepam |
| 247 | Zolmitriptan | Hyoscine |
| 248 | Zolmitriptan | Dexamethasone |
| 249 | Zolmitriptan | Emetrol |
| 250 | Zolmitriptan | Propofol |
| 251 | Eletriptan | Promethazine |
| 252 | Eletriptan | Aprepitant |
| 253 | Eletriptan | Dronabinol |
| 254 | Eletriptan | Perphenazine |
| 255 | Eletriptan | Palonosetron |
| 256 | Eletriptan | Trimethyobenzamide |
| 257 | Eletriptan | Metoclopromide |
| 258 | Eletriptan | Domperidone |
| 259 | Eletriptan | Prochlorperazine |
| 260 | Eletriptan | Chlorpromazine |
| 261 | Eletriptan | Trimethobenzamide |
| 262 | Eletriptan | Ondansetron |
| 263 | Eletriptan | Granisetron |
| 264 | Eletriptan | Hydroxyzine |
| 265 | Eletriptan | Acetylleucine Monoethanolamine |
| 266 | Eletriptan | Alizapride |
| 267 | Eletriptan | Azasetron |
| 268 | Eletriptan | Benzquinamide |
| 269 | Eletriptan | Bietanautine |
| 270 | Eletriptan | Bromopride |
| 271 | Eletriptan | Buclizine |
| 272 | Eletriptan | Clebopride |
| 273 | Eletriptan | Cyclizine |
| 274 | Eletriptan | Dimenhydrinate |
| 275 | Eletriptan | Diphenidol |
| 276 | Eletriptan | Dolasetron |
| 277 | Eletriptan | Meclizine |
| 278 | Eletriptan | Methallatal |
| 279 | Eletriptan | Metopimazine |
| 280 | Eletriptan | Nabilone |
| 281 | Eletriptan | Oxyperndyl |
| 282 | Eletriptan | Pipamazine |
| 283 | Eletriptan | Scopolamine |
| 284 | Eletriptan | Sulpiride |
| 285 | Eletriptan | Tetrahydrocannabinol |
| 286 | Eletriptan | Thiethylperazine |
| 287 | Eletriptan | Thioproperazine |
| 288 | Eletriptan | Tropisetron |
| 289 | Eletriptan | Droperidol |
| 290 | Eletriptan | Haloperidol |
| 291 | Eletriptan | Prochloperazine |
| 292 | Eletriptan | Metoclopramide |
| 293 | Eletriptan | Diphenhydramine |
| 294 | Eletriptan | Cannabis |
| 295 | Eletriptan | Midazolam |
| 296 | Eletriptan | Lorazepam |
| 297 | Eletriptan | Hyoscine |
| 298 | Eletriptan | Dexamethasone |
| 299 | Eletriptan | Emetrol |
| 300 | Eletriptan | Propofol |
| 301 | Naratriptan | Promethazine |
| 302 | Naratriptan | Aprepitant |
| 303 | Naratriptan | Dronabinol |
| 304 | Naratriptan | Perphenazine |
| 305 | Naratriptan | Palonosetron |
| 306 | Naratriptan | Trimethyobenzamide |
| 307 | Naratriptan | Metoclopromide |
| 308 | Naratriptan | Domperidone |
| 309 | Naratriptan | Prochlorperazine |
| 310 | Naratriptan | Chlorpromazine |
| 311 | Naratriptan | Trimethobenzamide |
| 312 | Naratriptan | Ondansetron |
| 313 | Naratriptan | Granisetron |
| 314 | Naratriptan | Hydroxyzine |
| 315 | Naratriptan | Acetylleucine Monoethanolamine |
| 316 | Naratriptan | Alizapride |
| 317 | Naratriptan | Azasetron |
| 318 | Naratriptan | Benzquinamide |
| 319 | Naratriptan | Bietanautine |
| 320 | Naratriptan | Bromopride |
| 321 | Naratriptan | Buclizine |
| 322 | Naratriptan | Clebopride |
| 323 | Naratriptan | Cyclizine |
| 324 | Naratriptan | Dimenhydrinate |
| 325 | Naratriptan | Diphenidol |
| 326 | Naratriptan | Dolasetron |
| 327 | Naratriptan | Meclizine |
| 328 | Naratriptan | Methallatal |
| 329 | Naratriptan | Metopimazine |
| 330 | Naratriptan | Nabilone |
| 331 | Naratriptan | Oxyperndyl |
| 332 | Naratriptan | Pipamazine |
| 333 | Naratriptan | Scopolamine |
| 334 | Naratriptan | Sulpiride |
| 335 | Naratriptan | Tetrahydrocannabinol |
| 336 | Naratriptan | Thiethylperazine |
| 337 | Naratriptan | Thioproperazine |
| 338 | Naratriptan | Tropisetron |
| 339 | Naratriptan | Droperidol |
| 340 | Naratriptan | Haloperidol |
| 341 | Naratriptan | Prochloperazine |
| 342 | Naratriptan | Metoclopramide |
| 343 | Naratriptan | Diphenhydramine |
| 344 | Naratriptan | Cannabis |
| 345 | Naratriptan | Midazolam |
| 346 | Naratriptan | Lorazepam |
| 347 | Naratriptan | Hyoscine |
| 348 | Naratriptan | Dexamethasone |
| 349 | Naratriptan | Emetrol |
| 350 | Naratriptan | Propofol |

As to any pharmaceutically active agent disclosed in the foregoing Table 16, it should be noted that any pharmaceutically acceptable salt of the recited pharmaceutically active agent is contemplated for use in the present invention. Furthermore, non-limiting examples of such pharmaceutically acceptable salts are disclosed herein.

While particular embodiments described herein have been shown and described herein, such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition comprising:
a plurality of first particulates comprising a 5HT_{1B} receptor agonist or a pharmaceutically acceptable salt thereof; and
a plurality of second particulates comprising an antiemetic or a pharmaceutically acceptable salt thereof,
wherein a weight ratio of the plurality of first particulates to the plurality of second particulates is of from about 3:1 to about 5:1.

2. The pharmaceutical composition of claim 1, wherein a weight ratio of the 5HT_{1B} receptor agonist or a pharmaceutically acceptable salt thereof to the antiemetic or a pharmaceutically acceptable salt thereof is of from about 1:2 to about 15:1.

3. The pharmaceutical composition of claim 2, wherein the weight ratio of the 5HT_{1B} receptor agonist or a pharmaceutically acceptable salt thereof to the antiemetic or a pharmaceutically acceptable salt thereof is of from about 3:2 to about 11:1.

4. The pharmaceutical composition of claim 3, wherein the weight ratio of the 5HT_{1B} receptor agonist or a pharmaceutically acceptable salt thereof to the antiemetic or a pharmaceutically acceptable salt thereof is of from about 3:1 to about 7:1.

5. The pharmaceutical composition of any one of claims 1 to 4, wherein the weight ratio of the 5HT_{1B} receptor agonist or a pharmaceutically acceptable salt thereof to the antiemetic or a pharmaceutically acceptable salt thereof is of from about 9:2 to about 11:2.

6. The pharmaceutical composition of any one of claims 1 to 5, wherein the weight ratio of the 5HT_{1B} receptor agonist or a pharmaceutically acceptable salt thereof to the antiemetic or a pharmaceutically acceptable salt thereof is about 5:1.

7. The pharmaceutical composition of any one of claims 1 to 6, wherein the weight ratio of the plurality of first particulates to the plurality of second particulates is of from about 3.5:1 to about 4.5:1.

8. The pharmaceutical composition of any one of claims 1 to 7, wherein the weight ratio of the plurality of first particulates to the plurality of second particulates is about 4:1.

9. The pharmaceutical composition of any one of claims 1 to 8, wherein a weight ratio of the 5HT_{1B} receptor agonist or a pharmaceutically acceptable salt thereof to a total weight of the plurality of first particulates is of from about 2:5 to about 7:10.

10. The pharmaceutical composition of any one of claims 1 to 9, wherein the 5HT_{1B} receptor agonist or a pharmaceutically acceptable salt thereof is present in an amount of about 61% by weight of the plurality of first particulates.

11. The pharmaceutical composition of any one of claims 1 to 10, wherein a weight ratio of the antiemetic or a pharmaceutically acceptable salt thereof to a total weight of the plurality of second particulates is of from about 2:5 to about 3:5.

12. The pharmaceutical composition of any one of claims 1 to 11, wherein the antiemetic or a pharmaceutically acceptable salt thereof is present in an amount of about 50% by weight of the plurality of second particulates.

13. The pharmaceutical composition of any one of claims 1 to 12, wherein the plurality of first particulates comprises one or more first pharmaceutically acceptable excipients and a weight ratio of a total amount of the 5HT_{1B} receptor agonist or pharmaceutically acceptable salt thereof to a total amount of the one or more first pharmaceutically acceptable excipients is of from about 2:1 to about 1:1.

14. The pharmaceutical composition of any one of claims 1 to 13, wherein the plurality of first particulates comprises one or more first pharmaceutically acceptable excipients and a weight ratio of the total amount of the 5HT_{1B} receptor agonist or pharmaceutically acceptable salt thereof to the total amount of the one or more first pharmaceutically acceptable excipients is about 3:2.

15. The pharmaceutical composition of any one of claims 1 to 14, wherein the plurality of second particulates comprises one or more second pharmaceutically acceptable excipients, and a weight ratio of a total amount of the antiemetic or a pharmaceutically acceptable salt thereof to a total amount of the one or more second pharmaceutically acceptable excipients is of from about 2:1 to about 1:2.

16. The pharmaceutical composition of any one of claims 1 to 15, wherein the plurality of second particulates comprises one or more second pharmaceutically acceptable excipients, and a weight ratio of the total amount of the antiemetic or a pharmaceutically acceptable salt thereof to the total amount of the one or more second pharmaceutically acceptable excipients is about 1:1.

17. A pharmaceutical composition comprising:
a plurality of first particulates comprising a 5HT_{1B} receptor agonist or a pharmaceutically acceptable salt thereof; and
a plurality of second particulates comprising an antiemetic or a pharmaceutically acceptable salt thereof,
wherein at least about 80% of both the 5HT_{1B} receptor agonist or a pharmaceutically acceptable salt thereof and the antiemetic or a pharmaceutically acceptable salt thereof are released within about 15 minutes as measured by contact of the pharmaceutical composition with dissolution fluid in a USP Apparatus 1 (Basket) rotating at 100 rpm.

18. The pharmaceutical composition of any one of claims 1 to 17, wherein at least about 80% of both the 5HT_{1B} receptor agonist or a pharmaceutically acceptable salt thereof and the antiemetic or a pharmaceutically acceptable salt thereof are released within about 30 minutes as measured by contact of the pharmaceutical composition with a dissolution fluid in a USP Apparatus 1 (Basket) rotating at 100 rpm.

19. The pharmaceutical composition of any one of claims 1 to 18, wherein the antiemetic or a pharmaceutically acceptable salt thereof has about the same release rate as that of the 5HT_{1B} receptor agonist or a pharmaceutically acceptable salt thereof.

20. The pharmaceutical composition of any one of claims 1 to 18, wherein the antiemetic or a pharmaceutically acceptable salt thereof has a slower release rate than the release rate of the 5HT_{1B} receptor agonist or a pharmaceutically acceptable salt thereof.

21. The pharmaceutical composition of any one of claims 1 to 20, wherein the antiemetic or a pharmaceutically acceptable salt thereof has a slower release rate than the release rate of the 5HT_{1B} receptor agonist or a pharmaceutically acceptable salt thereof within about 5 minutes as measured by contact of the pharmaceutical composition with a dissolution fluid in a USP Apparatus 1 (Basket) rotating at 100 rpm.

22. The pharmaceutical composition of any one of claims 1 to 21, wherein about 60% to about 65% of the antiemetic or a pharmaceutically acceptable salt thereof is released within about 5 minutes and about 70% to about 75% of the 5HT_{1B} receptor agonist or a pharmaceutically acceptable salt thereof is released within about 5 minutes as measured by contact of the pharmaceutical composition with a dissolution fluid in a USP Apparatus 1 (Basket) rotating at 100 rpm.

23. The pharmaceutical composition of any one of claims 1 to 22, wherein the pharmaceutical composition is a fast release pharmaceutical composition.

24. A pharmaceutical composition comprising:
a plurality of first particulates comprising a 5HT_{1B} receptor agonist or a pharmaceutically acceptable salt thereof; and
a plurality of second particulates comprising an antiemetic or a pharmaceutically acceptable salt thereof,
wherein about 90% to about 100% of the 5HT1B receptor agonist or a pharmaceutically acceptable salt thereof is stable for at least 30 days as measured by HPLC, and about 90% to about 100% of the antiemetic or a pharmaceutically acceptable salt thereof is stable for at least 30 days as measured by HPLC.

25. The pharmaceutical composition of any one of claims 1 to 24, wherein about 90% to about 100% of the 5HT_{1B} receptor agonist or a pharmaceutically acceptable salt thereof is stable for at least 90 days.

26. The pharmaceutical composition of any one of claims 1 to 25, wherein about 95% of the 5HT_{1B} receptor agonist or a pharmaceutically acceptable salt thereof is stable for at least 30 days.

27. The pharmaceutical composition of any one of claims 1 to 26, wherein about 90% to about 100% of the antiemetic or a pharmaceutically acceptable salt thereof is stable for at least 90 days.

28. The pharmaceutical composition of any one of claims 1 to 27, wherein about 100% of the antiemetic or a pharmaceutically acceptable salt thereof is stable for at least 30 days.

29. The pharmaceutical composition of any one of claims 1 to 28, wherein a diameter of each of the first particulates is from about 595 microns to about 1190 microns.

30. The pharmaceutical composition of any one of claims 1 to 29, wherein a diameter of each of the second particulates is from about 595 microns to about 1190 microns.

31. The pharmaceutical composition of any one of claims 1 to 30, wherein the 5HT_{1B} receptor agonist or a pharmaceutically acceptable salt thereof comprises a triptan or a pharmaceutically acceptable salt thereof.

32. The pharmaceutical composition of claim 31, wherein the triptan or a pharmaceutically acceptable salt thereof comprises sumatriptan, almotriptan, frovatriptan, eletriptan, rizatriptan, naratriptan, or a pharmaceutically acceptable salt thereof.

33. The pharmaceutical composition of claim 31 or 32, wherein the triptan or a pharmaceutically acceptable salt thereof comprises the sumatriptan or a pharmaceutically acceptable salt thereof.

34. The pharmaceutical composition of claim 33, wherein the sumatriptan or a pharmaceutically acceptable salt thereof is present in an amount therapeutically equivalent to about 25 mg to about 100 mg of sumatriptan.

35. The pharmaceutical composition of any one of claim 33 or 34, wherein the sumatriptan or a pharmaceutically acceptable salt thereof is present in an amount therapeutically equivalent to about 90 mg of sumatriptan.

36. The pharmaceutical composition of any one of claims 33 to 35, wherein the pharmaceutically acceptable salt of the sumatriptan comprises sumatriptan succinate.

37. The pharmaceutical composition of claim 36, wherein the sumatriptan succinate is present in an amount of from about 35 mg to about 140 mg.

38. The pharmaceutical composition of claim 36 or 37, wherein the sumatriptan succinate is present in an amount of about 126 mg.

39. The pharmaceutical composition of any one of claims 1 to 38, wherein the antiemetic or a pharmaceutically acceptable salt thereof comprises promethazine, ondansetron, aprepitant, dronabinol, perphenazine, palonosetron, trimethyobenzamide, metoclopromide, domperidone, prochlorperazine, chlorpromazine, trimethobenzamide, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, droperidol, haloperidol, prochloperazine, metoclopramide, diphenhydramine, cannabis, midazolam, lorazepam, hyoscine, dexamethasone, emetrol, propofol, or a pharmaceutically acceptable salt thereof.

40. The pharmaceutical composition of any one of claims 1 to 39, wherein the antiemetic or a pharmaceutically acceptable salt thereof comprises the promethazine or a pharmaceutically acceptable salt thereof.

41. The pharmaceutical composition of claim 39 or 40, wherein the promethazine or a pharmaceutically acceptable salt thereof is present in an amount therapeutically equivalent to about 22 mg of promethazine.

42. The pharmaceutical composition of any one of claims 39 to 41, wherein the pharmaceutically acceptable salt of promethazine comprises promethazine hydrochloride.

43. The pharmaceutical composition of claim 42, wherein the promethazine hydrochloride is present in an amount of from about 5 to about 50 mg.

44. The pharmaceutical composition of claim 42 or 43, wherein the promethazine hydrochloride is present in an amount of about 25 mg.

45. The pharmaceutical composition of any one of claims 1 to 44, wherein the plurality of first particulates comprises one or more first pharmaceutically acceptable excipients, wherein the one or more first pharmaceutically acceptable excipients comprises a diluent, binder, disintegrant or lubricant.

46. The pharmaceutical composition of claim 45, wherein:
the diluent comprises microcrystalline cellulose;
the binder comprises polyvinylpyrrolidone;
the disintegrant comprises croscarmellose sodium; or
the lubricant comprises magnesium stearate or talc.

47. The pharmaceutical composition of any one of claims 1 to 46, wherein the plurality of second particulates comprises one or more first pharmaceutically acceptable excipients, wherein the one or more first pharmaceutically acceptable excipients comprises a diluent or a disintegrant.

48. The pharmaceutical composition of claim 47, wherein: the diluent comprises microcrystalline cellulose; or the disintegrant comprises croscarmellose sodium.

49. The pharmaceutical composition of any one of claims 1 to 48, wherein:
the plurality of first particulates comprises:
about 50-150 mg of the 5HT_{1B} receptor agonist or a pharmaceutically acceptable salt thereof;
about 1-10 mg of polyvinylpyrrolidone;
about 50-100 mg of microcrystalline cellulose;
about 1-10 mg of croscarmellose sodium;
about 0.1-5 mg of magnesium stearate; and
a coating material; and
the plurality of second particulates comprises:
about 10-50 mg of antiemetic or a pharmaceutically acceptable salt thereof;
about 10-50 mg of microcrystalline cellulose;
about 0.1-5 mg of croscarmellose sodium; and
a coating material.

50. The pharmaceutical composition of any one of claims 1 to 49, wherein:
the plurality of first particulates comprises:
about 90 mg of sumatriptan or a therapeutically equivalent amount of pharmaceutically acceptable salt thereof;
about 4 mg of polyvinylpyrrolidone;
about 69 mg of microcrystalline cellulose;
about 4 mg of croscarmellose sodium;
about 1 mg of magnesium stearate; and
a coating material, wherein the coating material comprises polyvinyl alcohol; and
the plurality of second particulates comprises:
about 22 mg of promethazine or a therapeutically equivalent amount of pharmaceutically acceptable salt thereof;
about 24 mg of microcrystalline cellulose;
about 1 mg of croscarmellose sodium; and
a coating material, wherein the coating material comprises polyvinyl alcohol.

51. The pharmaceutical composition of any one of claims 1 to 48, wherein the first particulates comprise a coating material.

52. The pharmaceutical composition of any one of claim 1 to 48 or 51, wherein the second particulates comprise a coating material.

53. The pharmaceutical composition of claim 51 or 52, wherein the coating material is applied to the plurality of first particulates or the plurality of second particulates at a weight gain of from about 0.5% to about 5%.

54. The pharmaceutical composition of any one of claims 51 to 53, wherein the coating material is applied to the plurality of first particulates or the plurality of second particulates at a weight gain of about 2%.

55. The pharmaceutical composition of any one of claims 51 to 54, wherein the first particulates and the second particulates comprise the same coating material.

56. The pharmaceutical composition of any one of claims 51 to 55, wherein the coating material comprises polyvinyl alcohol, cellulose acetate phthalate, polyvinyl acetate phthalate, methacrylic acid copolymer, cellulose acetate trimellitate, hydroxypropyl methylcellulose phthalate, hydroxypropyl methylcellulose, hydroxypropyl methyl cellulose acetate succinate, shellac, sodium alginate, or zein.

57. The pharmaceutical composition of any one of claims 51 to 56, wherein the coating material comprises polyvinyl alcohol.

58. The pharmaceutical composition of any one of claims 51 to 57, wherein the coating material is polyvinyl alcohol.

59. The pharmaceutical composition of any one of claims 1 to 58, wherein:
i) a weight ratio of the plurality of first particulates to the plurality of second particulates is of from about 3:1 to about 5:1;
ii) at least about 80% of both the 5HT_{1B} receptor agonist or a pharmaceutically acceptable salt thereof and the antiemetic or a pharmaceutically acceptable salt thereof are released within about 15 minutes as measured by contact of the pharmaceutical composition with dissolution fluid in a USP Apparatus 1 (Basket) rotating at 100 rpm; and
iii) about 90% to about 100% of the 5HT1B receptor agonist or a pharmaceutically acceptable salt thereof is stable for at least 30 days as measured by HPLC, and about 90% to about 100% of the antiemetic or a pharmaceutically acceptable salt thereof is stable for at least 30 days as measured by HPLC.

60. An oral dosage form comprising a pharmaceutical composition of any one of claims 1 to 59.

61. A capsule comprising a pharmaceutical composition of any one of claims 1 to 59.

62. A pharmaceutical composition of any one of claims 1 to 59 for use in treatment of a headache in a subject in need thereof.

63. The pharmaceutical composition for use according to claim 62, wherein the treatment of the headache is acute or prophylactic.

64. The pharmaceutical composition for use according to claim 62 or 63, wherein the headache is a migraine headache.

65. The pharmaceutical composition for use according to any one of claims 62 to 64, wherein the headache is an acute migraine headache or a chronic migraine headache.

66. The pharmaceutical composition for use according to claim 64 or 65, wherein the headache is a migraine headache with or without an aura.

67. The pharmaceutical composition for use according to any one of claims 62 to 66, wherein the headache is a cluster headache.

68. A pharmaceutical composition of any one of claims 1 to 59 for use in treatment of a photophobia in a subject in need thereof.

69. The pharmaceutical composition for use according to claim 68, wherein the treatment of the photophobia is acute or prophylactic.

70. The pharmaceutical composition for use according to claim 68 or 69, wherein the pharmaceutical composition is used for treatment of a light sensitivity.

71. The pharmaceutical composition for use according to any one of claims 62 to 70, wherein the pharmaceutical composition is used for treatment of nausea or vomiting.

72. The pharmaceutical composition for use according to any one of claims 62 to 71, wherein the pharmaceutical composition is used for treatment of nausea associated with a headache or vomiting associated with a headache.

73. The pharmaceutical composition for use according to any one of claims 62 to 71, wherein the pharmaceutical composition is used for treatment of nausea associated with a headache and vomiting associated with a headache.

74. The pharmaceutical composition for use according to any one of claims 62 to 73, wherein a dosage of the pharmaceutical composition comprises about 25 mg to about 100 mg of sumatriptan.

75. The pharmaceutical composition for use according to any one of claims 62 to 73, wherein a dosage of the pharmaceutical composition comprises about 50 mg to about 75 mg of sumatriptan.

76. The pharmaceutical composition for use according to any one of claims 62 to 73, wherein a dosage of the pharmaceutical composition comprises about 50 mg to about 100 mg of sumatriptan.

77. The pharmaceutical composition for use according to any one of claims 62 to 76, wherein the pharmaceutical composition is suitable for use at one, two, or three times daily.

78. The pharmaceutical composition for use according to any one of claims 62 to 77, wherein the pharmaceutical composition is suitable for use at about every 8 to about every 12 hours.

79. The pharmaceutical composition for use according to any one of claims 62 to 78, wherein a second dose of the pharmaceutical composition is used after response to a first dose in a subject.

80. The pharmaceutical composition for use according to any one of claims 62 to 79, wherein doses after a first dose of the pharmaceutical composition are separated by at least 2 hours.

81. The pharmaceutical composition for use according to any one of claims 62 to 80, wherein a maximum dose of the pharmaceutical composition over a 24 hour period does not exceed 200 mg.

82. The pharmaceutical composition for use according to claim 81, wherein a maximum single dose of the pharmaceutical composition does not exceed 50 mg in a subject with mild to moderate hepatic impairment.

83. A method of treating a headache in a subject in need thereof, comprising administering to the subject a pharmaceutical composition of any one of claims 1 to 59.

84. The method of claim 83, wherein the treatment of the headache is acute or prophylactic.

85. The method of claim 83 or 84, wherein the headache is a migraine headache.

86. The method of claim 83 or 84, wherein the headache is an acute migraine headache or a chronic migraine headache.

87. The method of claim 85 or 86, wherein the headache is a migraine headache with or without an aura.

88. The method of any one of claims 83 to 87, wherein the headache is a cluster headache.

89. A method of treating a photophobia in a subject in need thereof, comprising administering to the subject a pharmaceutical composition of any one of claims 1 to 59.

90. The method of claim 89, wherein the treatment of the photophobia is acute or prophylactic.

91. The method of claim 89 or 90, wherein the pharmaceutical composition is used for treatment of a light sensitivity.

92. The method of any one of claims 83 to 91, wherein the pharmaceutical composition treats nausea or vomiting.

93. The method of any one of claims 83 to 91, wherein the pharmaceutical composition treats nausea associated with a headache or vomiting associated with a headache.

94. The method of any one of claims 83 to 91, wherein the pharmaceutical composition treats nausea associated with a headache and vomiting associated with a headache.

95. The method of any one of claims 83 to 94, wherein the administering comprises delivery of about 25 mg to about 100 mg of sumatriptan.

96. The method of any one of claims 83 to 94, wherein the administering delivers about 50 mg to about 75 mg of sumatriptan.

97. The method of any one of claims 83 to 94, wherein the administering delivers about 50 mg to about 100 mg of sumatriptan.

98. The method of any one of claims 83 to 97, wherein the administering is one, two, or three times daily.

99. The method of any one of claims 83 to 98, wherein the administering is about every 8 to about every 12 hours.

100. The method of any one of claims 83 to 99, wherein a second dose of the pharmaceutical composition is administered after response to a first dose in the subject.

101. The method of any one of claims 83 to 100, wherein doses after a first dose of the pharmaceutical composition are separated by at least 2 hours.

102. The method of any one of claims 83 to 101, wherein a maximum dose of the pharmaceutical composition over a 24 hour period does not exceed 200 mg.

103. The method of claim 102, wherein a maximum single dose of the pharmaceutical composition does not exceed 50 mg in a subject with mild to moderate hepatic impairment.
